(19) United States
(12) Patent Application Publication
(10) Pub. No.: US 2025/0306039 A1
(43) Pub. Date: Oct. 2, 2025

(54) PHOSPHO-TAU AGGREGATION-BASED BIOMARKERS FOR ALZHEIMER'S DISEASE DIAGNOSIS, DIFFERENTIATION, AND TREATMENT

(71) Applicants: NORTH CAROLINA CENTRAL UNIVERSITY, Durham, NC (US); DUKE UNIVERSITY, Durham, NC (US)

(72) Inventors: Bin XU, Chapel Hill, NC (US); Ling WU, Chapel Hill, NC (US); Shih-Hsiu WANG, Cary, NC (US); John F. Ervin, Durham, NC (US); Andrew J. Liu, Durham, NC (US)

(21) Appl. No.: 19/125,991

(22) Filed: Apr. 30, 2025

Related U.S. Application Data
(63) Continuation-in-part of application No. PCT/US2023/036372, filed on Oct. 31, 2023.
(60) Provisional application No. 63/381,609, filed on Oct. 31, 2022.
(60) Provisional application No. 63/641,577, filed on May 2, 2024.

(51) Int. Cl. G01N 33/68

(52) U.S. Cl. CPC G01N 33/6896; G01N 2440/14; G01N 2800/2814; G01N 2800/2821; G01N 2800/52

(57) ABSTRACT

Provided are methods of phospho-tau aggregation-based biomarker discovery, and new utilities for discovered biomarkers in Alzheimer's disease (AD) diagnosis, differentiation, and treatment. Novel p-tau sites, p-tau198, p-tauS356, p-tau396, and p-tau422, identified through such methods showed comparable or superior characteristics with established p-tau biomarkers, and identified biomarkers were capable of differentiating AD or mild cognitive impairment (MCI) from cognitively normal controls.

### CROSS-REFERENCE TO RELATED APPLICATIONS

This is a bypass continuation-in-part application of International Application No. PCT/US2023/036372, filed Oct. 31, 2023, which claims priority to and the benefit of U.S. Provisional Patent Application No. 63/381,609, filed Oct. 31, 2022, and which claims priority to and the benefit of U.S. Provisional Patent Application No. 63/641,577, filed May 2, 2024, the disclosures of each of which are incorporated herein by reference in their entireties.

### STATEMENT REGARDING FEDERAL SUPPORT

This invention was made in part with government support under grant numbers P30AG072958 and R01AG067607 awarded by National Institutes of Health. The government has certain rights in the invention.

### FIELD OF THE INVENTION

The present inventive concept relates to methods of phospho-tau aggregation-based biomarker discovery and new utilities of discovered biomarkers for Alzheimer's disease (AD) diagnosis, differentiation, and treatment.

### BACKGROUND

Extracellular Aβ amyloid plaques and intraneuronal tau neurofibrillary tangles are hallmark features in the brains of Alzheimer's disease (AD) patients.^{1,2} Until recently, plaques and tangles were thought not only to represent molecular signatures of AD, but also to cause the synaptic dysfunction and neuronal loss that lead to the memory and cognitive impairment characteristic of AD patients.^{3,4} Multiple lines of evidence have suggested that pathological changes in tangles correlate better with neuronal dysfunction than Aβ deposits.⁵⁻⁷ Moreover, a close relationship between tau aggregates and neuronal loss is well-established in the hippocampus and cerebral cortex.⁸ Tau aggregates are not only present in AD brains, but also in multiple neurodegenerative diseases known as “tauopathies”,⁹⁻¹⁰ including Pick's disease (PiD), progressive supranuclear palsy (PSP), and corticobasal degeneration (CBD). Transmission of tau pathology in AD and related tauopathies is believed to result through “prion-like” mechanisms that yield intercellular spreading of toxic protein aggregates. Tau hyperphosphorylation has been identified as a major contributing factor in forming pathogenic neurofibrillary tangles.¹¹

Classically, definitive diagnosis of AD relies on post-mortem neuropathological examination and confirmation of the presence of both Aβ plaques and neurofibrillary tau tangles. Upon recent development, clinical diagnosis of AD and AD-related dementia (ADRD) is supported by imaging biomarkers such as positron emission tomography, which is relatively expensive for patients, or by CSF biomarkers such as Aβ42, Aβ42/40 ratio, phosphorylated tau, and total tau which involves invasive spinal lumbar puncture. Identifying biomarkers for the development of noninvasive or minimally invasive and inexpensive testing across AD and ADRD is an urgent and unmet need. Significant progress has been made in the last few years in AD diagnosis, such as the development of tests for site-specific phospho-tau biomarkers (p-tau181 and p-tau217) to differentiate AD from non-AD with brain tissues, CSF, and blood samples.¹²⁻¹⁴ However, more biomarkers are needed because currently there are no well-established biomarkers available for early AD diagnosis such as mild cognitive impairment (MCI), which is the stage between typical cognitive decline of normal aging and more serious decline of dementia. Furthermore, additional diagnostic tools are needed because of the complexity of molecular pathology of AD such as heterogenous tau posttranslational modification (PTM) profiles in relation to misfolded tau and disease progression.¹⁵⁻¹⁶ Recent technological advances such as cryo-EM and advanced mass spectrometry-based proteomics provided multiple high-resolution structures of tau filaments directly extracted from AD and other tauopathy brains¹⁶⁻¹⁹ and a comprehensive map of PTM of misfolded tau in human AD patients and non-AD control subjects.¹⁵ These important advances together with information of high patient frequency sites of tau PTMs generated opportunities for developing new site-specific PTM-based tau biomarkers.

### SUMMARY

Aspects of the inventive concept relate to the discovery of “smear”-like high-molecular-weight (HMW), AD-specific tau aggregation species in postmortem brains detected by total tau antibodies. Initial differential detection led to a systematic screening of postmortem AD, rare tauopathies, MCI and normal control brain tissues with a comprehensive panel of site-specific phospho-tau antibodies covering nearly all high-patient frequency (>50%) tau phosphorylation sites with Western blotting analyses. Several novel p-tau sites were identified, such as those described in the Example section. The diagnostic performance of novel biomarkers, p-tau198, p-tau212/214, p-tau262/263, p-tauS356, p-tau396, and p-tau422 were further characterized and compared with established p-tau biomarkers p-tau181 and p-tau217. Aspects of the inventive concept provide an avenue for new biomarker discovery for not only AD and ADRD diagnosis, differentiation, and prognostication, but also for sensitive detection in broader neurodegenerative diseases, such as Parkinson's, amyotrophic lateral sclerosis, Huntington's, where posttranslational modifications of the hallmark amyloidogenic proteins may play important roles in disease pathogenesis and progression.

Aspects of the present inventive concept include analysis of the extent of phosphorylation and/or hyperphosphorylation of p-tau biomarkers, e.g., p-tau198, p-tau212, p-tau214, p-tau262, p-tau263, p-tauS356, p-tau396, and p-tau422, either individually, or in combination, e.g., p-tau212/214 and p-tau262/263.

According to an aspect of the inventive concept, provided is a method of diagnosing a neurodegenerative disease/disorder in a subject comprising: determining an extent of phosphorylation and/or hyperphosphorylation of at least one phospho-tau (p-tau) biomarker in the subject; and diagnosing whether the subject is afflicted with the neurodegenerative disease/disorder if the extent of phosphorylation and/or hyperphosphorylation of the p-tau biomarker exceeds a threshold indicative of a presence of the neurodegenerative disease/disorder.

According to another aspect of the inventive concept, provided is a method of differentiating a neurodegenerative disease/disorder in a subject comprising: determining an extent of phosphorylation and/or hyperphosphorylation of at least one p-tau biomarker; and differentiating whether the subject is afflicted with the neurodegenerative disease/disorder based on if the extent of phosphorylation and/or hyperphosphorylation of the p-tau biomarker exceeds a threshold indicative of the neurodegenerative disease/disorder.

According to another aspect of the inventive concept, provided is a method of diagnosing mild cognitive impairment (MCI) in a subject comprising: determining an extent of phosphorylation and/or hyperphosphorylation of at least one p-tau biomarker; and diagnosing whether the subject is afflicted with MCI if the extent of phosphorylation and/or hyperphosphorylation of the p-tau biomarker exceeds a threshold indicative of the subject being afflicted with MCI.

According to another aspect of the inventive concept, provided is a for differentiating MCI from normal cognitive decline in a subject comprising: determining an extent of phosphorylation and/or hyperphosphorylation of the p-tau biomarker; and differentiating whether the subject is afflicted with MCI from normal cognitive decline based on if the extent of phosphorylation and/or hyperphosphorylation of the p-tau biomarker exceeds a threshold indicative of the subject being afflicted with MCI.

Also provided by methods of the inventive concept are methods of treating neurodegenerative diseases/disorders, and methods of treating MCI.

### BRIEF DESCRIPTION OF THE DRAWINGS

FIG. 1. Western blotting analyses of control experiments demonstrating primary antibody specific-recognition of protein bands in our Western blotting screening experiments (FIGS. 2 and 4). Only mouse secondary antibody (panel A) or rabbit secondary antibody (panel B) were used. Same amount of specified brain homogenates of control (CTL) or AD postmortem tissues was used, as demonstrated by loading control Western blotting using anti-GAPDH or anti-β-actin antibodies.

FIG. 2. Western blot analysis of postmortem control, AD, and rare tauopathy subject brain tissues with tau antibodies. Comparison of brain homogenates of AD and sex/age-matched control subjects (CTL) (Panel A), or AD versus rare tauopathies PSP, CBD, and Pick's disease (Panel B) detected by Tau-5 anti-tau antibody. Comparison of brain homogenates of AD and sex/age-matched control subjects (CTL) (Panel C), or AD versus rare tauopathies PSP, CBD, and Pick's disease (Panel D) detected by A1103 anti-tau antibody. GAPDH or β-actin were used as loading controls. Differential detection of “smear”-like high-molecular-weight tau aggregates are highlighted with rectangles.

FIG. 3. 2N4R Tau phosphorylation frequencies in AD patient brain tissues. Only significant AD patient frequency sites (>10%) are shown. Data used for this figure were from Wesseling et al., 2022.¹⁵ The phosphorylation sites used for antibody screening in this study (T181, S198, S199, S202, S205, T212, S214, T217, T231, S235, S262, T263, S396, S304, and S422) are highlighted.

FIG. 4. Western blotting analyses of postmortem control, AD, and rare tauopathies brain tissues with site-specific phospho-tau (p-tau) antibodies. Tau phosphorylation sites with high AD patient frequency were selected and results are shown in panels A-H for each site. The left panels show side-by-side comparison of brain homogenates of control (CTL) and AD subjects analyzed by specified site-specific p-tau antibody, and the right panels show side-by-side comparison of brain homogenates of AD and rare tauopathy PSP, CBD, and PiD subjects analyzed by the same site-specific p-tau antibody. Molecular weight standards (in kD) are shown on the left side of each blot. Differences in high-molecular-weight soluble aggregates or others are highlighted in rectangles with dotted lines separating CTL from AD samples (in left panels) or AD subjects from rare tauopathies samples (in right panels). Loading controls by western blotting with either anti-GAPDH or anti-actin antibodies are shown below each panel.

FIG. 5. Western blotting analyses of postmortem control, AD, and rare tauopathies brain tissues with site-specific phospho-tau (p-tau) antibodies. Tau phosphorylation sites with high AD patient frequency were selected and results are shown in panels for each site. (Panel A) Side-by-side comparison of brain homogenates of control (CTL) and AD subjects analyzed by specified site-specific p-tau antibodies. (Panel B) Side-by-side comparison of brain homogenates of AD and rare tauopathy PSP, CBD, and PiD subjects analyzed by site-specific p-tau antibodies. Representative western blotting images are shown in corresponding FIG. 4. Individual western blotting experiment had been repeated at least twice.

FIG. 6. Performance of the ELISA assays based on identified p-tau198 site in discriminating AD from controls or related rare tauopathy subjects. Left panels are scattered plots showing postmortem brain tissue p-tau levels in Control (n=10), AD (n=18), PiD (n=5), CBD (n=9), and PSP (n=10) subjects. Corresponding bar graphs are shown in the middle panel. Statistical p values are indicated by asterisks (*p<0.05, ** p<0.01, ***p<0.001, and****p<0.0001). ROC curves were plotted for indicating trade-off sensitivity and specificity. All samples were run in singlicates.

FIG. 7. Performance of the ELISA assays based on identified p-tau198 site in discriminating mild cognitive impairment (MCI) (n=14) from cognitively normal brains (n=19). Upper panels are scattered plots showing p-tau levels in MCI and cognitively normal post-mortem brains. Lower panels show ROC curves for corresponding p-tau markers. Statistical p values are indicated by asterisks (*p<0.05; **p<0.01). ROC curves were plotted for indicating trade-off sensitivity and specificity. All samples were run in singlicates. Demographic characteristics of MCI patients and cognitively normal subjects are shown in Table 1.

FIG. 8. Comparative/correlation analyses of APOE4 carriers vs. non carriers in relation to p-tau198 levels, PMI vs. p-tau198 levels, or age vs. p-tau198 levels for the cases of cognitively normal and MCI subjects. (Panel A) Student t-test was used for analyzing genotypic APOE4 carriers or non-carriers effect on p-tau198 levels in GraphPad Prism 9.3.1. No significant difference was found between E4 carrier vs. non-carrier (p=0.20). (Panels B, C) Nonparametric Spearman's correlation coefficients were calculated using GraphPad Prism 9.3.1. PMI vs. p-taul98 level plot (Panel B) yields ρ of −0.24 (95% CI −0.5712 to 0.1566). Age vs. p-tau198 level plot (Panel C) yields ρ of −0.38 (95% CI −0.6556 to −0.008344).

FIG. 9. Performance of the ELISA assays based on identified p-tau198 site in discriminating mild cognitive impairment (MCI) from cognitively normal (CN) brains. Points with arrows identify the MCI subjects with Braak stage VI diagnosis.

FIG. 10. Posttranslational modification phosphorylation sites in archetypal amyloidogenic proteins tau, α-synuclein, and TDP-43, respective hallmark proteins in AD, Parkinson's and ALS neurodegenerative diseases. Other posttranslational sites such as acetylation, ubiquitination are not shown. For the tau phosphorylation sites shown in the format of 2N4R tau, only sites with >10% AD patient frequency are shown.¹⁵

FIGS. 11A and 11B. Western blotting analyses of postmortem control and AD brain tissues with site-specific phospho-tau (p-tau) antibodies and semi-quantification of western blotting intensity/area of the immunoreactivity of the HMW phospho-tau aggregate bands. (FIG. 11A) Selected tau phosphorylation sites with high AD patient frequency are shown. Each panel shows a side-by-side comparison of brain homogenates of control (CTL) and AD subjects analyzed by specified site-specific p-tau antibodies. Molecular weight standards (in kD) are shown on the left side of each blot. Differences in high-molecular-weight soluble aggregates or others are highlighted in red rectangles, with dotted lines separating CTL from AD samples. Loading controls by western blotting with either anti-GAPDH or anti-actin antibodies are shown below each panel. (FIG. 11B) Semi-quantitation of western blotting HMW phospho-tau aggregate bands were analyzed by densitometry using Odyssey Western blot detection. Side-by-side comparison of HMW p-tau aggregate bands from brain homogenates of control (CTL) and AD subjects analyzed by specified site-specific p-tau antibodies and measured by densitometry. Corresponding western blotting images are shown in panel A. The individual western blotting experiment had been repeated at least twice. All the background-subtracted density values were normalized with the loading control (GAPDH or β-actin). Fold increase of HMW phospho-tau aggregate band intensity in AD cases over controls with selected p-tau epitopes are indicated by red or blue arrows.

FIG. 12. Performance of the ELISA assays based on the identified p-tau396 site in discriminating AD from controls or related rare tauopathy subjects. (Panels A-D) Left: scatter plots show postmortem brain tissue p-tau levels in Control (n=10) and AD (n=18) subjects. Statistical p values are indicated by asterisks (****p<0.0001). Fold increase of the mean values from site-specific p-tau epitope detection in AD group vs. Control group are specified by red or blue arrows/texts in each scattered plot. Right: ROC curves plotted for indicating trade-off sensitivity and specificity for each site-specific p-tau biomarker. All samples were run in singlicates.

FIG. 13. Performance of the ELISA assays based on the identified p-tau396 site in discriminating MCI (n=22) from cognitively normal brains (n=16). (Panels A-D) Left: scatter plots showing p-tau levels in MCI and cognitively normal post-mortem brains. MCI cases in the scattered plots in all panels are color-coded, indicating different Braak stage for each case. Color code definitions are specified in the side bar in panel A. Right: ROC curves for corresponding p-tau markers. Statistical p values are indicated (p<0.05 or NS indicates not statistically significant). ROC curves were plotted for indicating trade-off sensitivity and specificity. All samples were run in singlicates.

FIG. 14. Representative images of the hippocampus CA4, CA3, CA2, CA1, subiculum, transentorhinal cortex (TErC), occipitotemporal cortex (OTC), and superior temporal cortex (STC) from participants with normal cognition (Panel A) and mild cognitive impairment (Panel B) immunostained for p-tau396. All images were taken from participants with Braak stage III neurofibrillary tangle pathology. A microscopic field with the highest density of neurofibrillary tangles in each brain region is selected for imaging. P-tau396 immunostaining shows denser tau pathology in the CA4, CA3, CA1, subiculum, OTC, and STC from subjects with MCI. Scale bar=100 mM in A and B.

FIG. 15 Representative whole slide-scanned images of the hippocampus immunostained for p-tau396 from participants that are cognitively normal (Panel A) and MCI (Panel B) analyzed with Visiopharm Oncotopix Discovery software. The hippocampus is manually segmented into CA4, CA3, CA2, CA1, subiculum. Whole slide-scanned images are zoomed in to show the CA1 region from a cognitively normal (Panel C) and MCI (Panel D) subject. Representative images (Panels E and F) demonstrate that the software can reliably detect p-tau396-positive pixels (depicted in red) after training with a Bayesian linear regression algorithm. Panel E shows the same field as panel C, and panel F shows the same field as panel D. The ratio of p-tau396-positive pixels and total pixels of each region of interest (ROI) is calculated to indicate tau burden. P-tau396 immunostaining demonstrates a higher tau burden in the CA4, CA3, CA1, subiculum, TErC, OTC, and STC from subjects with MCI (G). The difference in CA2 was not statistically significant. N=14 for cognitively normal, and N=10 for MCI.

FIG. 16. P-tau396-derived tau burden was calculated in cognitively normal and MCI subjects that are Braak stage III-IV. P-tau396 immunostaining demonstrates a higher tau burden in the CA4, CA3, CA1, subiculum, OTC, and STC of participants with MCI. The difference in CA2 and TErC was not statistically significant. N=9 for cognitively normal, and N=6 for MCI.

FIG. 17. Performance of the ELISA assays based on identified p-tau422 epitope in discriminating AD from cognitively normal controls (CN). (Panels A-D) Left: scatter plots showing postmortem brain tissue p-tau levels in cognitively normal and AD subjects. Statistical p values are indicated by asterisks (****p<0.0001). Panel A shows an exceptional 23.7-fold increase of average p-tau422 levels in AD brains versus controls, whereas on panels B-D indicate 1.9- to 2.4-fold increase of other specified p-tau levels in AD versus control brains. Right: corresponding diagnostic performance of the ELISA assays. ROC curves plotted indicate trade-off sensitivity and specificity. All samples were run in singlicates.

FIG. 18. Performance of the ELISA assays based on identified p-tau422 epitope in discriminating mild cognitive impairment (MCI) from cognitively normal brains. (Panels A-D) Left: scatter plots showing p-tau levels in MCI and in cognitively normal postmortem brains. Statistical p values are indicated by asterisks (*p<0.05, **p<0.01) or NS (not significant, p>0.05). An exceptional 6.3-fold increase of average p-tau422 levels in MCI brains versus cognitively normal controls. Right: corresponding diagnostic performance of the ELISA assays. ROC curves were plotted for indicating trade-off sensitivity and specificity. All samples were run in singlicates.

FIG. 19. Representative images of the hippocampus CA4, CA3, CA2, CA1, subiculum, transentorhinal cortex (TErC), occipitotemporal (OTC), and superior temporal cortex (STC) from subjects with normal cognition (Panel A) and mild cognitive impairment (Panel B) immunostained for p-tau422. All images were taken from participants with Braak stage III neurofibrillary tangle pathology. P-tau422 immunostaining show denser tau pathology in the CA4, CA3, CA1, subiculum, OTC, and STC from subjects with MCI. Scale bar=100 mM in panels A and B.

FIG. 20. Representative whole slide-scanned images of the hippocampus immunostained for p-tau422 from participants that are cognitively normal (Panel A) and MCI (Panel B) analyzed with Visiopharm Oncotopix Discovery software. The software is trained to identify p-tau422-positive pixels using a Bayesian linear regression algorithm. The hippocampus is manually segmented into CA4, CA3, CA2, CA1, subiculum, and the ratio of p-tau422-positive pixels and total pixels of each region of interest (ROI) is calculated. P-tau422 immunostaining demonstrates a higher tau burden in the CA4, CA3, CA2, CA1, subiculum, TErC, OTC, and STC from subjects with MCI (Panel C). N=14 for cognitively normal, and N=10 for MCI.

FIG. 21. P-tau422-derived tau burden was calculated in cognitively normal and MCI subjects that are Braak stage III-IV. P-tau422 immunostaining demonstrates a higher tau burden in the CA4, CA3, CA2, CA1, subiculum, TErC, OTC, and STC of participants with MCI. N=8 for cognitively normal, and N=6 for MCI.

FIG. 22. Evidence of p-tau356, p-tau422, but not p-tau198, are markers of pre-tangles in AD. (Panel A) Representative images of the hippocampus CA1 from the same subject (Tha1 phase 4, Braak stage IV, CERAD score moderate) immunostained with AT8, p-tau198, p-tau356, and p-tau422. p: pretangles; i: intermediary 1; m: mature tangles. (Panel B) Analysis of tangle maturity levels show that p-tau356 and p-tau422 detects a higher percentage of pretangles. (Panels C, D) p-tau356 detects more pretangles in Braak stage III-VI subjects (Panel C), and p-tau422 detects more tangles in Braak IV-VI subjects (Panel D).

### DETAILED DESCRIPTION

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a” “an” and “the” are intended to include the plural forms as well, unless the context clearly indicates otherwise. Additionally, as used herein, the term “and/or” includes any and all combinations of one or more of the associated listed items and may be abbreviated as “/”.

The term “comprise,” as used herein, in addition to its regular meaning, may also include, and, in some embodiments, may specifically refer to the expressions “consist essentially of” and/or “consist of.” Thus, the expression “comprise” can also refer to, in some embodiments, the specifically listed elements of that which is claimed and does not include further elements, as well as embodiments in which the specifically listed elements of that which is claimed may and/or does encompass further elements, or embodiments in which the specifically listed elements of that which is claimed may encompass further elements that do not materially affect the basic and novel characteristic(s) of that which is claimed. For example, that which is claimed, such as a composition, formulation, method, system, etc. “comprising” listed elements also encompasses, for example, a composition, formulation, method, kit, etc. “consisting of,” i.e., wherein that which is claimed does not include further elements, and a composition, formulation, method, kit, etc. “consisting essentially of,” i.e., wherein that which is claimed may include further elements that do not materially affect the basic and novel characteristic(s) of that which is claimed.

The term “about” generally refers to a range of numeric values that one of skill in the art would consider equivalent to the recited numeric value or having the same function or result. For example, “about” may refer to a range that is within ±1%, ±2%, ±5%, ±10%, ±15%, or even ±20% of the indicated value, depending upon the numeric values that one of skill in the art would consider equivalent to the recited numeric value or having the same function or result. Furthermore, in some embodiments, a numeric value modified by the term “about” may also include a numeric value that is “exactly” the recited numeric value. In addition, any numeric value presented without modification will be appreciated to include numeric values “about” the recited numeric value, as well as include “exactly” the recited numeric value. Similarly, the term “substantially” means largely, but not wholly, the same form, manner or degree and the particular element will have a range of configurations as a person of ordinary skill in the art would consider as having the same function or result. When a particular element is expressed as an approximation by use of the term “substantially,” it will be understood that the particular element forms another embodiment.

Unless otherwise defined, all technical and scientific terms used herein have the same meaning as commonly understood by one of ordinary skill in the art to which this invention belongs.

Tau is a phosphorylated protein, containing 85 potential serine (S), threonine (T), and tyrosine (Y) phosphorylation sites. Many of the phosphorylated residues on tau are found in the proline-rich domain of tau, flanking the microtubule-binding domain. The phosphorylation status and isoform expression of tau are developmentally regulated and both phosphorylation status and isoform expression are important factors for cytoskeletal plasticity during embryogenesis and early development. In early developmental stages a single tau isoform, 0N3R, is expressed and tau phosphorylation is elevated relative to adult brain. In contrast, all six tau isoforms are present in normal mature human brain, and at this stage tau phosphorylation is relatively reduced.

Tau aggregates are present in multiple neurodegenerative diseases known as “tauopathies”, including, e.g., Alzheimer's disease (AD), Pick's disease (PiD), progressive supranuclear palsy (PSP), and corticobasal degeneration (CBD). Such misfolded tau aggregates are therefore potential sources for tauopathy biomarker discovery. Using a tau antibody screening approach targeting high-molecular-weight misfolded tau aggregates, several total tau antibodies and a comprehensive set of site-specific phosphor tau (p-tau) antibodies targeting tau phosphorylation sites with high AD patient frequencies were tested. Screens revealed that site-specific p-tau antibodies can not only differentiate AD from non-AD brains/subjects, but also discriminate AD from rare tauopathies PiD, PSP and CBD brains. Furthermore, these screens revealed that these site-specific p-tau antibodies can differentiate mild cognitive impairment (MCI), which in some, but not all cases, may lead to any of the neurodegenerative diseases/disorders noted herein, from non-MCI brains/subjects, e.g., those exhibiting cognitive decline as a result of normal aging. Differential detection of tau aggregates identified several novel p-tau sites as new biomarkers. Embodiments of the inventive concept include, for example, p-tau198, p-tau212/214, p-tau262/263, p-tau356, as well as p-tau396 and p-tau422, as novel AD biomarkers with excellent sensitivity and specificity comparable with or exceeding those of existing biomarkers p-tau181, p-tau217, and/or p-tau231. These results demonstrated that p-taul98, p-tau396, and/or p-tau422 detection and analysis can not only differentiate AD from non-AD controls, but also diagnose AD from related 4R tauopathies PSP and CBD with excellent diagnostic capability. Furthermore, p-tau198, or p-tau S198, p-tau356, p-tau396, and p-tau422 analysis were able to discriminate mild cognitive impairment (MCI) from cognitively normal brains with solid differentiation power. Additional AD biomarkers according to embodiments of the inventive concept suitable for analysis include, for example, p-tau212/214 (dual phosphorylation sites at positions 212 and 214), and p-tau262/263 (dual phosphorylation sites at positions 262 and 263). Embodiments of the present inventive concept provide a new avenue for diagnosis and differentiation tools for AD and related tauopathies, and discovery of the same.

The terms “phosphorylated tau,” “p-tau,” and “phospho-tau” refer to phosphorylated forms of tau protein. Tau proteins are a group of highly soluble protein isoforms produced by alternative splicing of the MAPT gene. Expression of the human MAPT gene results in six major tau isoforms in the adult human central nervous system and two isoforms in the peripheral nervous system. The brain-specific isoforms vary in the number of N-terminal inserts (0N, 1N, or 2N, i.e., 0, 1, or 2 N-terminal inserts) and C-terminal repeat domains (3R or 4R, i.e., 3 or 4 C-terminal inserts) due to alternative splicing of exons 2, 3, and 10, resulting in sizes between 48 kDa (0N3R) and 67 kDa (2N4R) of the corresponding proteins. The isoforms of phosphorylated and/or hyperphosphorylated tau proteins analyzed for the extent of phosphorylation and/or hyperphosphorylation, and which may be detected according to embodiments of the inventive concept are not particularly limited, and may be any of the isoforms found, for example, in samples derived from a subject, such as any of the six isoforms of tau found in humans, i.e., 0N3R, 1N3R, 2N3R, 0N4R, 1N4R, and 2N4R isoforms of tau. In some embodiments, the tau protein isoform, and phosphorylated forms thereof, analyzed and detected include human tau 2N4R (tau isoform 2, NCBI Reference Sequence NP_005901.2).

Phosphorylation sites of, for example, but not limited to, the tau 2N4R isoform, analyzed and used as biomarkers according to embodiments of the inventive concept include, for example, phosphorylation of serine-198 (S198), serine-199 (S199), serine-202 (S202), serine-214 (S214), serine-262 (S262), serine-356 (S356), serine-396 (S396), serine-404 (S404), serine-422 (S422), threonine-205 (T205), threonine-212 (T212), and/or threonine-263 (T263), or any combination of one or more thereof, of the tau 2N4R isoform, or may include the corresponding phosphorylation site or sites to those described herein for tau 2N4R isoform that are found on any of the other tau isoforms that may be present in a subject, as would be appreciated by one of skill in the art, without departing from the scope of the inventive concept. In some embodiments, the biomarker includes phosphorylation of S198. In some embodiments, the biomarker includes phosphorylation of any one of S198, S356, S396, and/or S422 of tau 2N4R, or any combination of one or more thereof, for example, T212/S214, and/or S262/T263, of tau 2N4R.

Determining, measuring, and/or analyzing of the phosphorylation status, frequencies, extents, and/or levels of phosphorylation (phosphorylated versus unphosphorylated) of a tau phosphorylation site used as a biomarker in a subject, and/or a sample from a subject according to embodiments of the inventive concept is not particularly limited, and the phosphorylation status/levels of phosphorylation/frequencies of phosphorylation/extents of phosphorylation for such biomarkers may be determined according to any method that may be appreciated by one of skill in the art without limitation.

In some embodiments, the phosphorylation status, frequencies, extents, and/or levels of phosphorylation (phosphorylated versus unphosphorylated) of a tau phosphorylation site used as a biomarker may be used as an indicator that a subject is afflicted with a neurodegenerative disease/disorder. The indicator may by if phosphorylation status, frequencies, extents, and/or levels of phosphorylation exceeds a threshold as would be appreciated by one of skill in the art as indicative of the presence of the neurodegenerative disease/disorder in the subject. For example, an increase in the frequency, extent, and/or level of phosphorylation of a tau phosphorylation site beyond a threshold may lead to a diagnosis indicative of the presence of a neurodegenerative disease/disorder, and/or that a subject is afflicted with a neurodegenerative disease/disorder.

“Subject” as used herein may be a patient. In some embodiments, the subject is a human; however, a subject of this disclosure can include an animal subject, particularly mammalian subjects such as canines, felines, bovines, caprines, equines, ovines, porcines, rodents (e.g., rats and mice), lagomorphs, primates (including non-human primates), etc., including domesticated animals, companion animals and wild animals for veterinary medicine, treatment or pharmaceutical drug development purposes.

The human subjects relevant to this disclosure may be male or female and may be any species and of any race or ethnicity, including, but not limited to, Caucasian, African-American, African, Asian, Hispanic, Indian, etc., and combined backgrounds. The subjects may be of any age, including newborn, neonate, infant, child, adolescent, adult, and geriatric.

In some embodiments, the human subject may be at risk for developing a neurodegenerative disease, such as any one of the diseases/disorders as described herein, e.g., AD, PiD, PSP, CBD and/or MCI. Risk factors for any one of these diseases/disorders include, but are not limited to, increasing age and individuals carrying the e4 variant of the APOE gene (APOE4), and other medical conditions/lifestyle factors, such as diabetes, smoking, elevated/high blood pressure, elevated cholesterol, obesity, depression, lack of physical exercise/activity, low education level, and/or infrequent participation in mentally/socially stimulating activities.

Having described various aspects of the present invention, the same will be explained in further detail in the following examples, which are included herein for illustration purposes only, and which are not intended to be limiting to the invention.

### Example 1

### Phospho-Tau Biomarkers

### Phospho-Tau Biomarker P-Tau S198

The phospho-tau biomarker p-tau S198 was identified from antibody screening assays as a site with outstanding contrasts of phosphorylated tau aggregates between AD versus normal control brains, and AD versus rare tauopathies brains. We therefore selected this site as an example for demonstration about its utilities. We quantified p-tau S198 levels in the brain homogenates of temporal cortex in the discovery cohort using standard ELISA assays. Brain p-tau S198 level significantly increased, over 2.3-fold in AD when compared with controls (2.303±0.107 vs. 0.971±0.172 relative units; p<0.0001). Brain p-tau S198 had an excellent diagnostic performance to identify AD cases from normal controls (AUC=0.96, CI_{95%}=0.90-1.00). P-taul98 not only readily identified AD cases from normal controls, but was also capable to discriminate AD from other tauopathies PSP and CBD. Brain p-tau S198 levels were significantly higher in AD (2.303±0.107 relative unit) than those in rare tauopathies PSP (2.303±0.107 vs. 0.907±0.240 relative units; p<0.001), and CBD (2.303±0.107 vs. 1.004±0.136 relative units; p<0.0001). Brain p-tau S198 also exhibited excellent diagnostic performance to identify AD from PSP and CBD; all these three diseases belong to 4R tauopathies (AD vs. PSP: AUC=0.98, CI_{95%}=0.94-1.00; AD vs. CBD: AUC=0.99, CI_{95%}=0.96-1.00).

In comparison to two known site-specific p-tau biomarkers T181 and T217, p-tau S198 showed comparable differentiation power with those of p-tau T217 but slightly better diagnostic performance than those of p-tau T181. P-tau217 showed differentiating power to identify AD from controls with AUC=0.98 and CI_{95%}=0.93-1.00. Average p-tau217 concentrations were 0.81±0.04 relative units in the AD brains and 0.33±0.05 relative units in the control brains, a 2.43-fold increase with a p value <0.0001. Ptau181 tests showed differentiating power to identify AD from controls with AUC=0.89 and CI_{95%}=0.76-1.00. Average p-tau181 concentrations were 2.58±0.17 relative units in the AD brains and 1.39±0.08 relative units in the control brains, a 1.86-fold increase with a p value <0.0001.

Most importantly, p-tau S198 is a promising new biomarker for MCI diagnosis (early-stage AD diagnosis). Brain p-tau198 levels significantly increased in MCI brains when compared with cognitively normal controls (1.18±0.15 vs. 0.75±0.11 relative units with a 1.57-fold increase and a p value <0.05). P-taul98 was capable to discriminate MCI cases from cognitively normal controls with an AUC value of 0.75 and CI_{95%}=0.58-0.92. To our knowledge, there are no well-established biomarkers to diagnosing MCI currently in clinical practice.

### Phospho-Tau Biomarker P-Tau T212/S214

The phospho-tau biomarker p-tau T212/S214 was identified from antibody screening assays as a site with outstanding contrasts of phosphorylated tau aggregates between AD versus normal control brains, and AD versus rare tauopathies brains. We therefore selected this site as an example for demonstration about its utilities. We quantified p-tau T212/S214 levels in the brain homogenates of temporal cortex in the discovery cohort using standard ELISA assays. Brain p-tau T212/S214 level dramatically increased, over 12-fold in AD when compared with controls (3.19±0.17 vs. 0.26±0.04 relative units with normalized total protein amount and a p value <0.0001). Brain p-tau T212/S214 had an exceptionally high diagnostic performance to identify AD cases from normal controls (AUC=1.00, CI_{95%}=1.00-1.00). P-tau212/214 not only readily identified AD cases from normal controls, but was also capable to discriminate AD from other tauopathies PiD, PSP and CBD. Brain p-tau S212/T214 levels were significantly higher in AD (3.19±0.17 relative unit with normalized total protein amount) than those in rare tauopathies PiD, CBD, and PSP (1.58±0.19, 0.78±0.14, 0.85±0.28 relative unit with normalized total protein amount). Brain p-tau T212/S214 also exhibited excellent diagnostic performance to identify AD from PiD, PSP and CBD (AD vs. PiD: AUC=0.96, CI_{95%}=0.87-1.00; AD vs. PSP: AUC=0.98, CI_{95%}=0.93-1.00; AD vs. CBD: AUC=0.99, CI_{95%}=0.97-1.00).

In comparison to three known site-specific p-tau biomarkers T181, T217, and T231, p-tau S212/T214 showed superior diagnostic performance, as described in the following multiple aspects: (1) p-tau181, p-tau217, and p-tau231 gave somewhat lower performance values to identify AD from controls: AUC=0.84, CI_{95%}=0.68-0.99 for T181; AUC=0.98, CI_{95%}=0.93-1.00 for T217; and AUC=0.94, CI_{95%}=0.86-1.00 for T231. (2) Furthermore, while p-tau levels of these three biomarkers measured from ELISA assays remained statistically higher in AD brains versus those of controls and those of rare tauopathies, fold of increase of p-tau aggregate levels in AD were much diminished when compared with those in controls and other tauopathies, particularly for the cases of p-tau181 and p-tau217. For example, p-tau181 levels were 2.44±0.18 relative unit, 1.39±0.08 relative unit, 1.07±0.13 relative unit, 0.81±0.08 relative unit, and 1.10±0.16 relative unit with normalized amount of total protein for AD, control, PiD, CBD, and PSP brains respectively. In comparison to 12.0-fold increase of p-tau212/214 levels in AD brains vs. controls, p-tau181 aggregates levels only increased to a modest 1.8-fold (p-tau217, p-tau231 aggregates levels increased by 2.4-fold or 2.0-fold respectively in AD brains vs. normal controls). Therefore p-tau212/214 has significantly better sensitivity in differentiating AD from non-AD controls. Similarly, p-tau212/214 biomarker has significantly better sensitivity in discriminating AD from rare tauopathies PSP or CBD, all of which belong to 4R-tauopathies.

Most significantly, p-tau T212/S214 showed discriminating capability to diagnose mild cognitive impairment (MCI) stage of AD from cognitively normal in ELISA tests of brain homogenates with AUC=0.86, CI_{95%}=0.68-1.00 and a p value <0.01 when MCI and cognitively normal cohorts were compared. Existing p-tau biomarkers, p-tau181 was not capable to diagnose MCI from cognitively normal cases in ELISA tests, and p-tau217 showed comparable discriminating capability to diagnose MCI from cognitively normal cases in ELISA tests with an AUC value of 0.85, CI_{95%}=0.71-0.99 and a p value <0.01.

### Phospho-Tau Biomarker P-Tau S262/T263

The phospho-tau biomarker p-tau S262/T263 was identified from antibody screening assays as a site with outstanding contrasts of phosphorylated tau aggregates between AD versus normal control brains, and AD versus rare tauopathies brains. We therefore selected this site as an example for demonstration about its utilities. Significantly, p-tau S262/T263 showed discriminating capability to diagnose mild cognitive impairment (MCI) stage of AD from cognitively normal in ELISA tests of brain homogenates with AUC=0.76, CI_{95%}=0.55-0.96 and a p value <0.05 when MCI and cognitively normal cohorts were compared.

### Phospho-Tau Biomarker P-Tau S356

The phospho-tau biomarker p-tau S356 was identified, and with antibody screening data, as a site with outstanding contrasts of phosphorylated tau aggregates between AD versus normal control brains, and AD versus rare tauopathies brains. We therefore selected this site as an example for proof-of-principle study for pre-clinical demonstration about its utilities. The level of p-tau 356 was quantified in the brain homogenates of temporal cortex in the discovery cohort using standard ELISA assays. Brain p-tau356 level significantly increased, over five-fold in AD when compared with controls (1.1±0.1 vs. 0.21±0.1 relative units; p<0.01). Brain p-tau356 had a high diagnostic performance to identify AD cases from normal controls (AUC=0.89, CI_{95%}=0.74-1.00).

More significantly, p-tau356 showed discriminating capability to diagnose mild cognitive impairment (MCI) stage of AD from cognitively normal in ELISA tests of brain homogenates with an outstanding AUC=0.93, CI95%=0.79-1.00 and p=0.002 when MCI and cognitively normal cohorts were compared. Such discrimination capability has been validated using immunohistochemistry staining of respective MCI and cognitively normal brains of 8 sublocations: p-tau356-derived tau burden were all significantly increased in MCI brains versus cognitively normal brains in hippocampal CA1-CA4, subiculum, entorhinal cortex, occipitotemporal cortex, and superior temporal cortex (p=0.0092, 0.0463, 0.0136, 0.0433, 0.0105, 0.0323, 0.0059, and 0.0025 respectively). Existing p-tau biomarkers p-tau181, p-tau217, p-tau231 were not robustly capable of diagnosing/differentiating MCI from cognitively normal cases in ELISA tests.

Another significant new utility is that p-tau356 biomarker is a superior neuropathological biomarker for Alzheimer's postmortem staging. The AT8 epitope (p-tau202/205) is the current reference marker as an immunostaining reagent for AD disease staging. The p-tau356 marker has shown significantly better Braak stage correlation (r=0.83) than the reference marker AT8 (r=0.67), when superior temporal cortex tissues are used for staining.

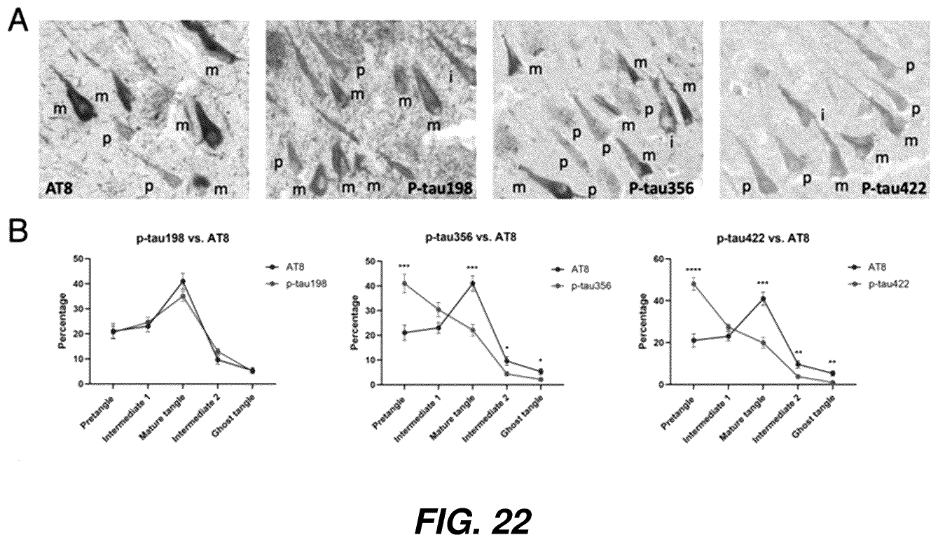

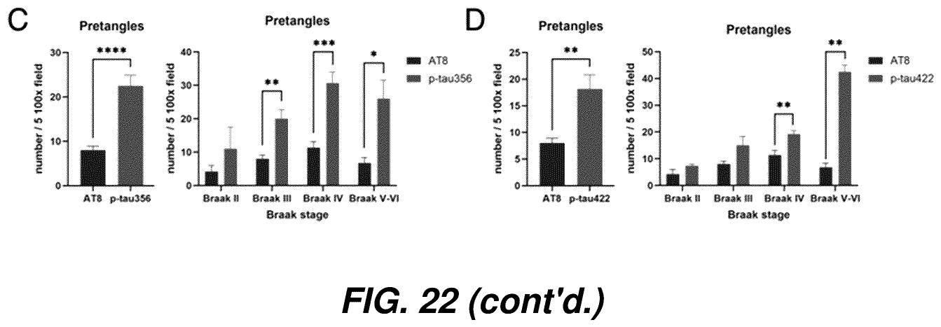

A further new utility is that the p-tau356 epitope can serve as a pretangle biomarker. Pretangles exhibit cytoplasmic tau immunoreactivity in neurons without apparent formation of fibrillary structures. Neurofibrillary tangle (NFT) maturity level (Moloney et al, 2021; Moloney et al, 2023) was analyzed in sections of the hippocampus CA1 immunostained with AT8 (p-tau202/205, a current neuropathological reference), p-tau198, and p-tau356, and discovered that p-tau356 label a higher percentage of pretangles compared to AT8 and p-tau198 (FIGS. 22, Panels A and B). Previous studies have shown that p-tau epitopes used as fluid biomarkers, such as p-tau181, p-tau217, and p-tau231, show a predilection for labeling NFTs at earlier maturity levels (Moloney et al, 2023). These results not only provide further rationale for developing p-tau356 as a biomarker for early-stage AD diagnosis. Further, p-tau356 detects significantly more pretangles than AT8 (FIGS. 22, Panels C and D), the current gold standard for neuropathological assessment of NFTs, suggesting that p-tau356 biomarker may provide a more accurate assessment of tau pathology across a broad NFT Braak stage of Alzheimer's disease.

A preliminary single molecule array (Simoa) test for p-tau356 has been developed, which facilitates measurement of and analysis for this novel biomarker in plasma or CSF samples. Further technical development of this biomarker for applications in biofluid sample analysis is underway.

The p-tauS356 biomarker has been tested in the brain tissues from postmortem Alzheimer's, MCI, and cognitively normal control subjects. The experimental validation data demonstrated that p-tauS356 is capable as a new biomarker for AD staging (differentiating MCI stage from cognitively normal stage), as well as being promising as a biomarker for early AD diagnosis.

### Phospho-Tau Biomarker P-Tau S396

The phospho-tau biomarker p-tau S396 was identified from antibody screening assays as a site with outstanding contrasts of phosphorylated tau aggregates between AD versus normal control brains, and AD versus rare tauopathies brains. We therefore selected this site as an example for demonstration about its utilities. We quantified p-tau S396 levels in the brain homogenates of temporal cortex in the discovery cohort using standard ELISA assays. Brain p-tau S396 level dramatically increased, over nine-fold in AD when compared with controls (870.5±113.8 vs. 92.9±15.0 pg/mg total protein; p<0.0001). Brain p-tau S396 had an outstandingly high diagnostic performance to identify AD cases from normal controls (AUC=0.98, CI_{95%}=0.95-1.00). P-tau396 not only readily identified AD cases from normal controls, but was also capable to discriminate AD from other tauopathies PSP and CBD. Brain p-tau S396 levels were significantly higher in AD (870.5±113.8 pg/mg total protein) than those in rare tauopathies CBD and PSP (121.9±17.9 and 118.3±40.2 pg/mg total protein respectively). Brain p-tau S396 also exhibited excellent diagnostic performance to identify AD from PSP and CBD; all these three diseases belong to 4R tauopathies (AD vs. PSP: AUC=0.97, CI_{95%}=0.91-1.00; AD vs. CBD: AUC=0.96, CI_{95%}=0.89-1.00).

In comparison to three known site-specific p-tau biomarkers T181, T217, and T231, p-tau S396 showed superior diagnostic performance, as described in the following multiple aspects: (1) p-tau181 and p-tau231 gave somewhat lower performance values to identify AD from controls: AUC=0.84, CI_{95%}=0.68-0.99 for T181 and AUC=0.94, CI_{95%}=0.86-1.00 for T231. P-tau217 has comparable diagnostic capability as those of p-tau396 to identify AD from controls: AUC=0.98, CI_{95%}=0.93-1.00. (2) Furthermore, while p-tau levels of these three biomarkers measured from ELISA assays remained statistically higher in AD brains versus those of controls and those of rare tauopathies, fold of increase of p-tau aggregate levels in AD were much diminished when compared with those in controls and other tauopathies, particularly for the cases of p-tau181 and p-tau217. For example, P-tau181 levels were 2.44±0.18 relative unit, 1.39±0.08 relative unit, 1.07±0.13 relative unit, 0.81±0.08 relative unit, and 1.10±0.16 relative for normalized amount of total protein for AD, control, PiD, CBD, and PSP brains respectively. In comparison to 9.4-fold increase of p-tau396 levels in AD brains vs. controls, p-tau181 aggregates levels only increased to a modest 1.8-fold (p-tau217, p-tau231 aggregates levels increased by 2.4-fold or 2.0-fold respectively in AD brains vs. normal controls). Therefore p-tau396 has significantly better sensitivity in differentiating AD from non-AD controls. Similarly, p-tau396 biomarker has better sensitivity in discriminating AD from rare tauopathies PSP or CBD, all of which belong to 4R-tauopathies.

Most significantly, p-tau S396 showed discriminating capability to diagnose mild cognitive impairment (MCI) stage of AD from cognitively normal in ELISA tests of brain homogenates with a robust AUC=0.84, CI_{95%}=0.68-0.99 and a p value <0.01 when MCI and cognitively normal cohorts were compared. Existing p-tau biomarkers p-tau181 and p-tau231 were not capable to diagnose MCI from cognitively normal cases in ELISA tests. p-tau217 showed comparable discriminating capability to diagnose MCI from cognitively normal cases in ELISA tests with an AUC value of 0.85, CI_{95%}=0.71-0.99 and a p value <0.01.

### Phospho-Tau Biomarker P-Tau S422

The phospho-tau biomarker p-tau S422 was identified from antibody screening assays as a site with one of the cleanest contrasts of phosphorylated tau aggregates between AD versus normal control brains, and AD versus rare tauopathies brains. We therefore selected this site as an example for demonstration about its utilities. We quantified p-tau S422 levels in the brain homogenates of temporal cortex in the discovery cohort using standard ELISA assays. Brain p-tau S422 level dramatically increased, with an exceptional 24.1-fold in AD when compared with controls (2.65±0.18 vs. 0.11±0.01 relative unit with normalized total protein amount; p<0.0001). Brain p-tau S422 achieved perfect diagnostic performance to identify AD cases from normal controls (AUC=1.00, CI_{95%}=1.00-1.00). P-tau422 not only readily identified AD cases from normal controls, but was also capable to discriminate AD from other tauopathies PiD, PSP and CBD. Brain p-tau S422 levels were significantly higher in AD (2.65±0.18 relative unit with normalized total protein amount) than those in rare tauopathies PiD, CBD, and PSP (0.53±0.09, 0.35±0.06, 0.61±0.23 units with normalized total protein amount). Brain p-tau S422 also exhibited outstanding diagnostic performance to identify AD from PiD, PSP and CBD (AD vs. PiD: AUC=0.99, CI_{95%}=0.95-1.00; AD vs. PSP: AUC=0.96, CI_{95%}=0.90-1.00; AD vs. CBD: AUC=1.00, CI_{95%}=1.00-1.00).

In comparison to three known site-specific p-tau biomarkers T181, T217, and T231, p-tau S422 showed superior diagnostic performance, as described in the following multiple aspects: (1) p-tau181, p-tau217, and p-tau231 gave somewhat lower performance values to identify AD from controls: AUC=0.84, CI_{95%}=0.68-0.99 for T181; AUC=0.98, CI_{95%}=0.93-1.00 for T217; and AUC=0.94, CI_{95%}=0.86-1.00 for T231. (2) Furthermore, while p-tau levels of these three biomarkers measured from ELISA assays remained statistically higher in AD brains versus those of controls and those of rare tauopathies, fold of increase of p-tau aggregate levels in AD were much diminished when compared with those in controls and other tauopathies, particularly for the cases of p-tau181 and p-tau217. For example, p-tau181 levels were 2.44±0.18 relative unit, 1.39±0.08 relative unit, 1.07±0.13 relative unit, 0.81±0.08 relative unit, and 1.10±0.16 relative for normalized amount of total protein for AD, control, PiD, CBD, and PSP brains respectively. In comparison to 23.4-fold increase of p-tau422 levels in AD brains vs. controls, p-tau181 aggregates levels only increased to a modest 1.8-fold (p-tau217, p-tau231 aggregates levels increased by 2.4-fold or 2.0-fold respectively in AD brains vs. normal controls). Therefore p-tau422 has significantly better sensitivity in differentiating AD from non-AD controls. Similarly, p-tau422 biomarker has significantly better sensitivity in discriminating AD from rare tauopathies PSP or CBD, all of which belong to 4R-tauopathies.

More significantly, p-tau S422 showed discriminating capability to diagnose mild cognitive impairment (MCI) stage of AD from cognitively normal in ELISA tests of brain homogenates with an outstanding AUC=0.90, CI_{95%}=0.78-1.00 with a p value <0.001 when MCI and cognitively normal cohorts were compared. Existing p-tau biomarkers p-tau181 and p-tau231 were not capable to diagnose MCI from cognitively normal cases in ELISA tests. p-tau217 showed slightly lower discriminating capability to diagnose MCI from cognitively normal cases in ELISA tests with an AUC value of 0.85, CI_{95%}=0.71-0.99 and a p value <0.01.

Furthermore, the p-tau S422 epitope can serve as a pretangle biomarker. Neurofibrillary tangle (NFT) maturity level (Moloney et al, 2021; Moloney et al, 2023) was analyzed in sections of the hippocampus CA1 immunostained with AT8 (p-tau202/205, a current neuropathological reference), p-tau198, and p-tau422, and discovered that p-tau422 label a higher percentage of pretangles compared to AT8 and p-tau198 (FIG. 22, Panels A and B). Previous studies have shown that p-tau epitopes used as fluid biomarkers, such as p-tau181, p-tau217, and p-tau231, show a predilection for labeling NFTs at earlier maturity levels (Moloney et al, 2023). These results not only provide further rationale for developing p-tau422 as biomarkers for early-stage AD diagnosis. Further, p-tau422 detects significantly more pretangles than AT8 (FIG. 22, Panels C and D), the current gold standard for neuropathological assessment of NFTs, suggesting that p-tau422 biomarker may provide a more accurate assessment of tau pathology across a broad NFT Braak stage of Alzheimer's disease.

### REFERENCES FOR EXAMPLE 1

Moloney C M, Lowe V J, Murray M E. (2021) Visualization of neurofibrillary tangle maturity in Alzheimer's disease: A clinicopathologic perspective for biomarker research. Alzheimers Dement 17(9):1554-1574.
Moloney C M, Labuzan S A, Crook J E, Siddiqui H, Castanedes-Casey M, Lachner C, Petersen R C, Duara R, Graff-Radford N R, Dickson D W, Mielke M M, Murray M E. (2023) Phosphorylated tau sites that are elevated in Alzheimer's disease fluid biomarkers are visualized in early neurofibrillary tangle maturity levels in the post mortem brain. Alzheimers Dement 19(3):1029-1040.

### Example 2

### Site-Specific Phospho-Tau Aggregation-Based Biomarker Discovery For Ad Diagnosis and Differentiation

### Methods

Postmortem Brain Tissue Collection. Postmortem brain tissues from non-AD controls, cognitively normal subjects, mild cognitive impairment (or mild impairment) subjects, AD dementia patients, and rare tauopathy patients were collected from the Bryan Brain Bank and Biorepository of the Duke University Medical Center Alzheimer's Disease Research Center (Duke/UNC ADRC). Demographics, NeuroStatus, Braak staging of AD-tau and other neuropathology diagnosis are described in detail in Tables 1 and 2. All participants were enrolled in the autopsy and brain donation program of the Joseph and Kathleen Pryce Bryan ADRC as previously described.⁴¹ All subjects gave informed consent. All protocols and the use of postmortem brain tissues for research were approved by the Duke University Institutional Review Board. Neuropathological evaluation was performed following published guidelines.⁴²⁻⁴⁵ Clinical diagnosis of dementia, MCI, and cognitively normal was based on clinical dementia rating (CDR). Consensus meetings were held annually to review and update the clinical diagnosis based on contemporaneous NIA-AA criteria.⁴¹

Brain Tissue Collection, Handling and Analysis. Brain tissues were homogenized at 10% (w/v) in lysis buff-er (10 mM Tris pH 7.4, 150 mM NaCl, 0.5% Nonidet P-40, 0.5% deoxycholate, 5 mM EDTA) with Mini-Bead Beater or hand-held homogenizer. The brain homogenates were further subjected to centrifugation at 1,000×g for 3 min at 4° C. and the supernatants were collected and stored at −80° C. for Western blotting and ELISA analyses (see below).

Western Blotting Analysis. Brain homogenates (10% w/v) were resuspended in 1× Laemmli sample buffer (no reducing agent, no heating), loaded onto 4-12% Bis-Tris NuPAGE (Invitrogen, Waltham, MA) or home-made12% Tris-Glycine gel. Proteins were transferred to Immobilon FL membrane (Millipore, Burlington, MA) for 30 min at 100 volts. The membranes were then blocked with LI-COR blocking buffer, and probed with the appropriate primary antibodies. Tau-5 (cat #AHB0042, 1:1000 dilution) and p-Tau-Thr181 mAb AT270 (cat #MN1050, 1:1000) were purchased from Invitrogen (Waltham, MA). P-tau198 rabbit mAb (cat #ab79540, 1:1000 dilution) and p-tau356 rabbit pAb (cat #ab75603, 1:1000 dilution) were purchased from abeam (Waltham, MA). Tau Rabbit pAb A1103 (cat #AP1103, 1:1000 dilution), p-tau202(cat #AP0765, 1:1000 dilution), p-tau205 rabbit pAb, (cat #AP0168, dilution 1:1000), p-tau217 rabbit pAb (cat #AP1233, 1:1000 dilution), p-tau231 Rabbit mAb (cat #AP0053, 1:1000 dilution), p-tau262 Rabbit pAb (cat #AP0397, 1:1000 dilution), and p-tau404 Rabbit pAb (cat #APO170, 1:1000 dilution) were obtained from ABclonal (Woburn, MA). Fluorescent labeled secondary antibodies, IRDye 680LT donkey anti-mouse IgG (cat #926-68022, LI-COR, Inc. Lincoln, NE) and IRDye 800CW Donkey anti-rabbit IgG (cat #926-32213 LI-COR, Inc. Lincoln, NE) were diluted to 1:50,000. The immunoreactivity was visualized by Odyssey Western blot detection (LICOR, Inc. Lincoln, NE). GAPDH or β-actin was used as loading control. Control Western blotting analyses were performed the same as regular analyses except that no primary anti-bodies were applied to confirm antibody-specific detection.

Semi-quantification of western blotting intensity/area of “smear” results was performed as the following. The immunoreactivity was visualized first and then HMW phospho-tau aggregate band quantification was analyzed by densitometry using Odyssey western blot detection software (LICOR, Inc., Lincoln, NE). During the quantification process, equal rectangle area was set for each sample for normalization. All the background-subtracted density values were normalized with loading control (GAPDH or β-actin). Western blotting exposure time was optimized to avoid signal oversaturation.

ELISA Measurements. Protein concentrations of the brain samples were measured by BCA assays (Pierce BCA protein assay kit, Thermo Fisher Scientific). 25 μg of total protein per well was used for ELISA assays according to manufacturer's instruction. ELISA kit for p-tau181(cat #KH00631) was purchased from Invitrogen (Waltham, MA) and p-tau217 ELISA kit (cat #59672) was procured from Cell Signaling (Danvers, MA). ELISA kit for p-tau198 was homebrew using Tau (Total) human ELISA kit (Invitrogen, cat #KHB0041) plate coated with total tau capture antibody and p-tau198 rabbit mAb (abcam; cat #ab79540) as detection antibody. The absorbance was measured at OD450 by SPECTRAmax plus plate reader (Molecular Devices, San Jose, CA).

Statistical Analysis. All data are presented as the mean±S.E.M and the differences were analyzed with unpaired Student's t test as implemented within GraphPad Prism software (version 6.0). p values <0.05 were considered significant. Relative quantification of p-tau198, p-tau181, and p-tau217 in AD patients and control subjects (AD vs. CTL) or AD and rare tauopathy subjects were compared using the statistical software GraphPad Prism 9. The specificity and sensitivity of tau with the specific phosphorylated sites in AD and CTL subjects, or AD and tauopathy subjects were determined based on the area under the curve (AUC) of receiver operating characteristics (ROC) analysis. The 95% confidence interval (CI_{95%}) of AUC was calculated with Wilson/Brown method.

Correlation Analysis. Genotypic APOE4 carriers or non-carriers in relation to p-tau198 ELISA levels were analyzed by t-test in GraphPad Prism 9.3.1. The nonparametric Spearman's correlation coefficients between postmortem interval (PMI) versus p-tau198 levels, or age versus p-Tau198 levels were calculated using GraphPad Prism 9.3.1. Pairs of datasets were input into GraphPad data table. Confidence level was set at 95%.

### Results

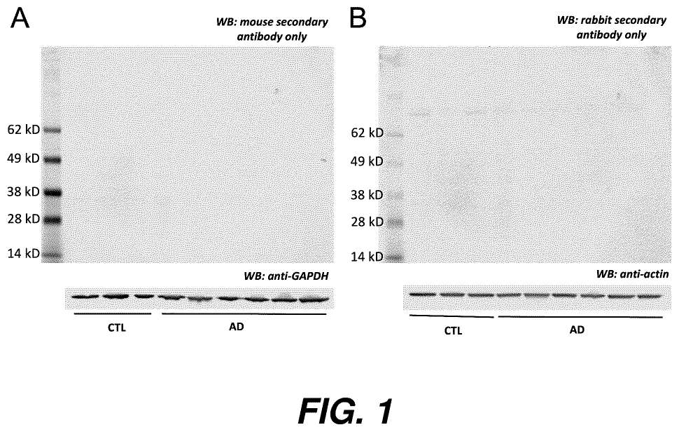

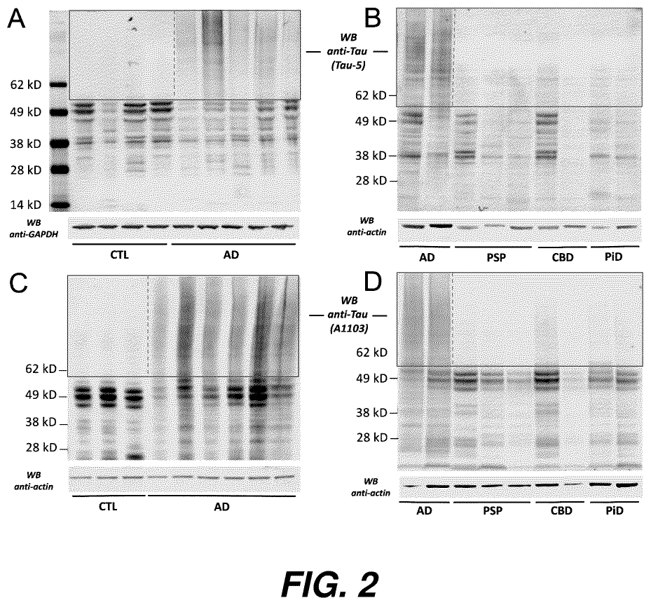

Differential Detection of Tau Aggregates in AD brains but not in the Controls. We first investigated the aggregated tau species patterns in postmortem AD brains and age-matching normal controls using two independent anti-tau antibodies: Tau-5 is a mouse monoclonal antibody, generated using amino acid 210-241 epitope of bovine tau as immunogen; A1103 is a rabbit polyclonal antibody that recognizes carboxy-terminal human tau sequence (amino acids 283-441 of 2N4R-tau sequence). With both antibody probes, AD brain tissues demonstrated clear “smear”-type of HMW, tau antibody-detectable species characteristic of typical protein aggregates (FIGS. 2A and 2C, highlighted with rectangles of AD samples), whereas there were very little or no such tau aggregates in the control samples (FIGS. 2A and 2C, highlighted with rectangles of CTL samples). Monomeric tau species as well as low-molecular-weight tau antibody-detectable species (likely partially degraded tau fragments) were observed from both AD and control brain tissues. Separately, we compared AD brain samples with those of rare tauopathies PSP, CBD, and PiD. Interestingly, rare tauopathy brain homogenates showed minimal aggregated tau species, in stark contrast with AD brain samples (FIGS. 2B and 2D, highlighted with rectangles). Control experiments demonstrated primary antibody-specific recognition of protein bands (FIG. 1). These initial differential detections led us to a systematic screening of postmortem AD, MCI, rare tauopathies, and normal control brain samples with a comprehensive panel of site-specific phospho-tau antibodies.

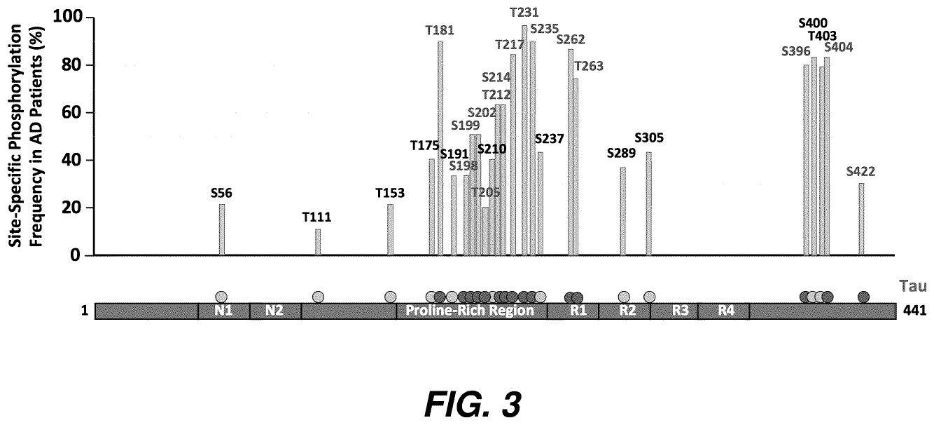

Identification of Novel Site-Specific Phospho-Tau Sites as Biomarkers. Selection of tau phosphorylation sites were primarily based on the high frequencies of these tau phosphorylation sites in AD patients from recent high-resolution quantitative proteomics map of PTMs on tau isoforms extracted from postmortem AD brain tissues,¹⁵ as well as available site-specific quality tau antibodies. Only significant AD patient frequency p-tau sites were chosen (patient frequency >10%; FIG. 3). More than twenty such p-tau sites were identified and over a dozen p-tau sites were selected (FIG. 3, highlighted) to screen the postmortem brain homogenates for HMW, site-specific p-tau antibody-detectable aggregated tau species with western blotting analysis. These sites include p-tau T181 and p-tau T217 sites that were recently being developed as AD biomarkers for diagnosis and/or tracking disease progression, therefore these sites served as validation controls (for an excellent review on phospho-tau AD biomarkers, see reference Leuzy et al., 2022).^{13,21-24}

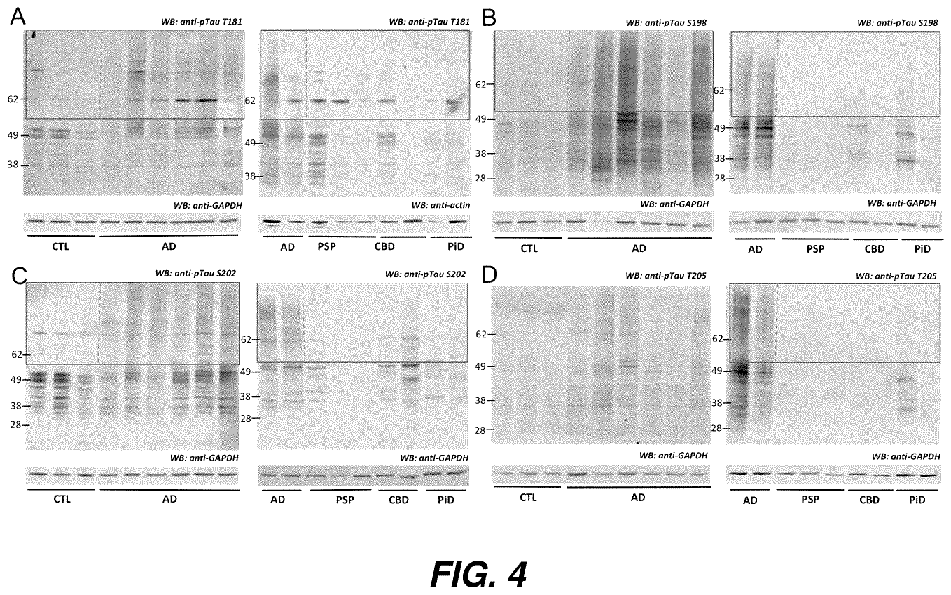

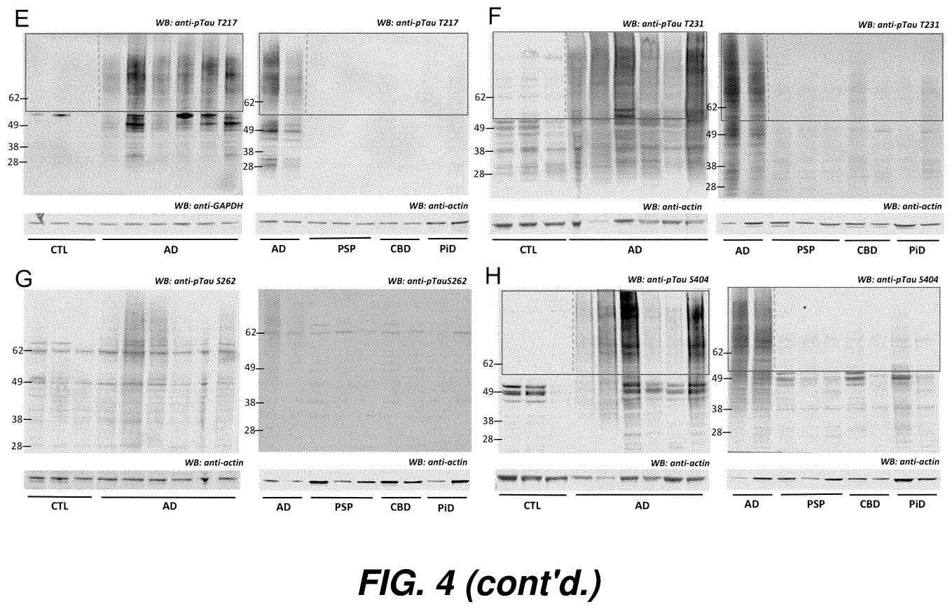

Similar to western blotting analyses of postmortem AD and control subject brain tissues with anti-tau antibodies, our search for candidate p-tau biomarkers was mainly based on the presence/absence of the HMW p-tau aggregate bands comparing AD brains with normal controls. As expected, p-tau T181 (FIG. 4A) and p-tau T217 (FIG. 4E) blots showed the presence of much stronger intensities of “smear-like” HMW protein aggregates from AD brains detected by respective site-specific p-tau antibodies (highlighted by the rectangles). Separate anti-GAPDH (or anti-actin) western analysis showed an approximate comparable amount of loading controls. In addition to p-tau181 and p-tau217 controls, we identified p-tau198, p-tau202, p-tau231, and p-tau404 sites (FIGS. 4B, 4C, 4F, and 4H) that differentiated AD brains from control tissues. However, p-tau205 and p-tau262 sites were not able to discriminate AD from normal controls (FIGS. 4D and 4G). We further compared AD brain samples with those from rare tauopathies. All these sites, except S262, had minimal or no HMW p-tau aggregates in rare tauopathies detected by each site-specific p-tau antibody, in clear contrast to those HMW aggregates in the AD brains (FIG. 4; highlighted in the rectangles in each of the right panels). To our knowledge, p-tau198, p-tau202, and p-tau404 have not been systematically evaluated as potential biomarkers to differentiate AD from normal controls and to differentiate AD from rare tauopathies. P-tau231 has been reported as a biomarker for AD diagnosis.^{14,21,25}

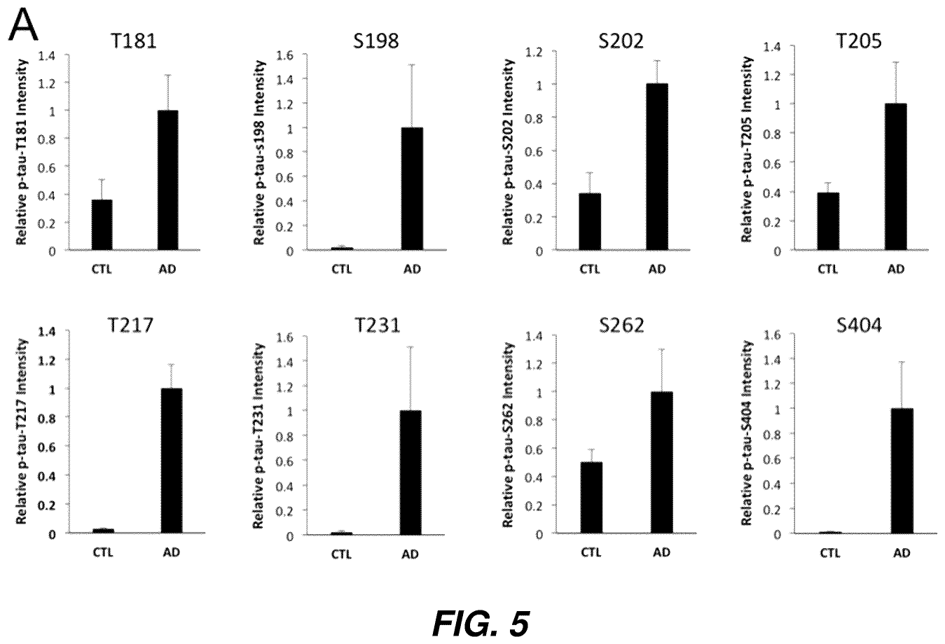

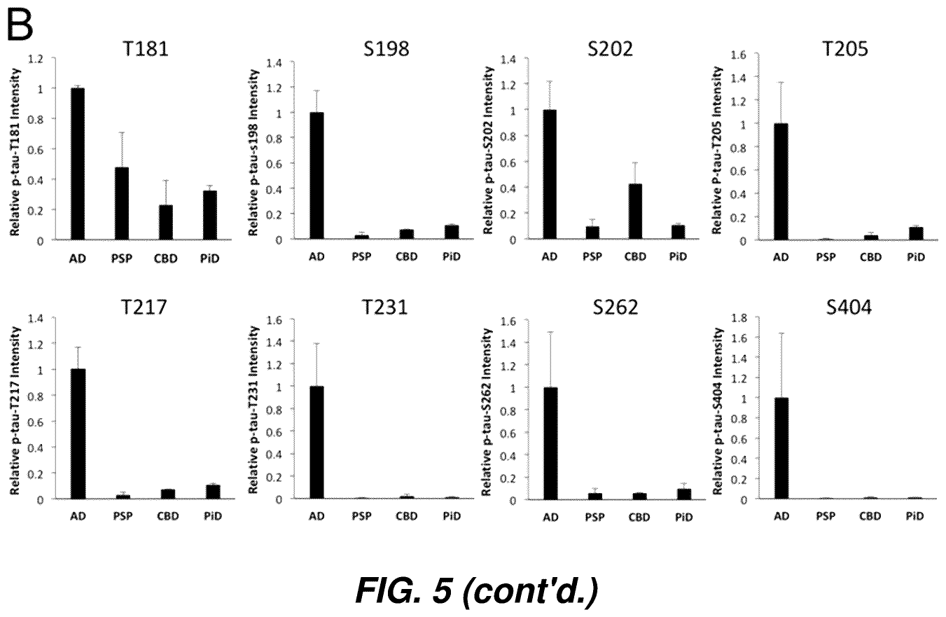

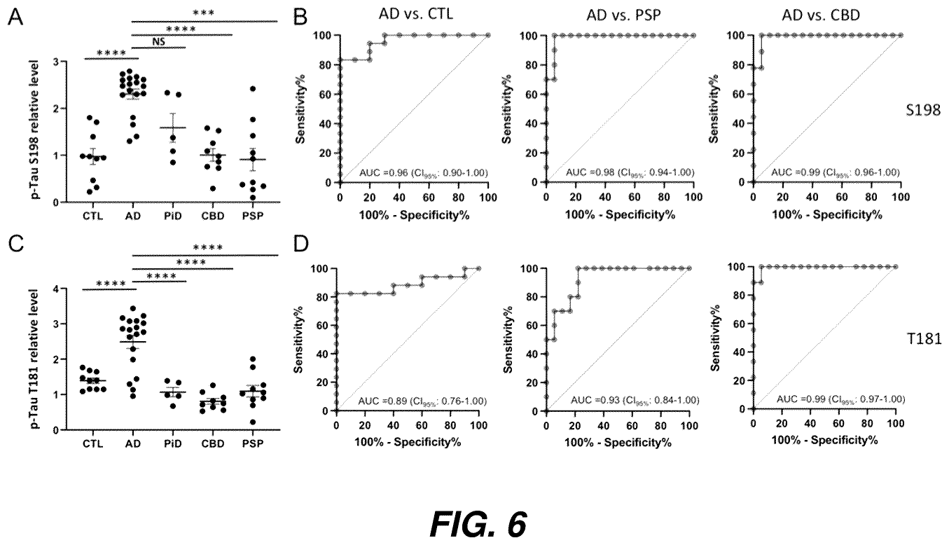

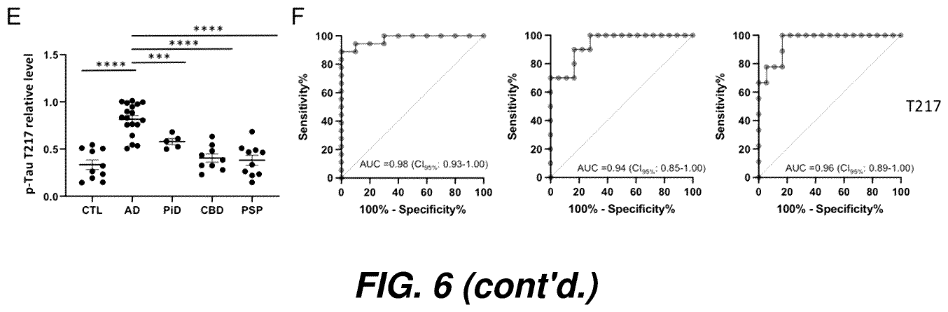

Phospho-tau S198 as a biomarker for AD diagnosis with differentiation power comparable with the existing p-tau biomarkers. P-taul98 was identified from our antibody screening as a site with one of the biggest contrasts of phosphorylated tau aggregates between AD and control brains, and AD versus rare tauopathies (FIG. 4B and FIG. 5). We therefore selected this site as a proof-of-principle example to demonstrate the feasibility of our approach for new biomarker discovery. We quantified p-tau198 levels in the brain homogenates of temporal cortex in the discovery cohort using ELISA assays. Demographic characteristics and neuropathological diagnosis of this cohort of AD, rare tauopathies, and non-AD patients are described in Tables 1 and 2. Brain p-tau198 levels significantly increased in AD when compared with controls (2.303±0.107 vs. 0.971±0.172 relative units with a 2.37-fold increase and a p value <0.0001; FIG. 6A). Diagnostic performance to identify AD cases from normal controls yields an AUC of 0.96 (CI_{95%}=0.90-1.00) (FIG. 5B). P-taul98 not only readily identified AD cases from normal controls, but also was capable of discriminating AD from PSP and CBD with differentiating power of AUC of 0.98 (CI₉₅%=0.94-1.00) for AD vs. PSP and AUC of 0.99 (CI₉₅%=0.96-1.00) for AD vs. CBD (FIGS. 6A and 6B). AD, PSP, and CBD all belong to 4R tauopathies.

We also compared p-tau198 with p-tau181 and p-tau217 side-by-side, two known biomarkers in current clinical use. P-taul98 showed comparable differentiating power with those of p-tau217 but slight better diagnostic performance than those of p-tau181, such as the AUC measures in AD vs. controls and AD vs. PSP discrimination tests (FIGS. 6C-6F). P-tau217 showed differentiating power to identify AD from controls with AUC=0.98 and CI_{95%}=0.93-1.00 (FIG. 6F) Average p-tau217 concentrations were 0.81±0.04 relative units in the AD brains and 0.33±0.05 relative units in the control brains, a 2.43-fold increase with a p value <0.0001 (FIG. 6E). P-tau181 tests showed differentiating power to identify AD from controls with AUC=0.89 and CI_{95%}=0.76-1.00 (FIG. 6D). Average p-tau181 concentrations were 2.58±0.17 relative units in the AD brains and 1.39±0.08 relative units in the control brains, a 1.86-fold increase with a p value <0.0001 (FIG. 6C). Due to low case number of PiD brains, we did not evaluate the diagnostic performance of various p-tau markers related to PiD cohorts.

Phospho-tau S198 as a new biomarker for MCI diagnosis. To test the potential of p-tau198 as a biomarker for more challenging MCI early AD diagnosis, we quantified p-tau198 levels in the brain homogenates of temporal cortex in the discovery cohorts of MCI and cognitively normal subjects using ELISA assays.

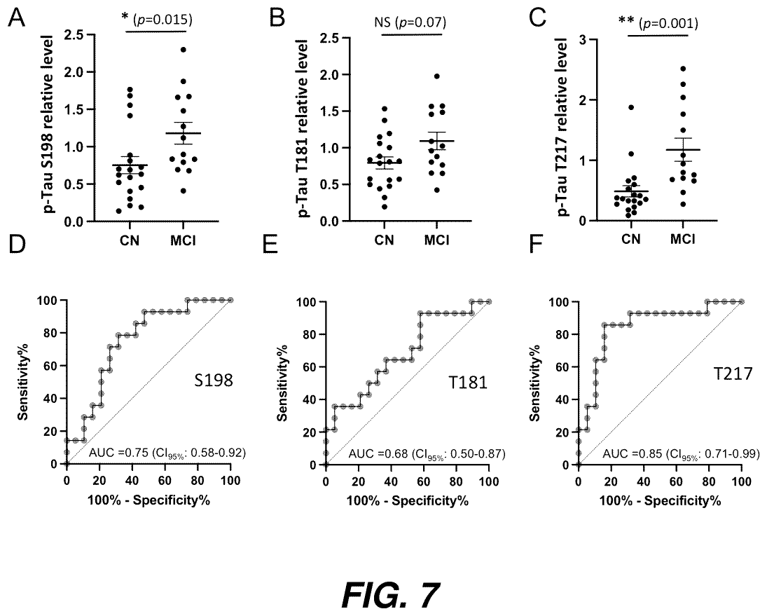

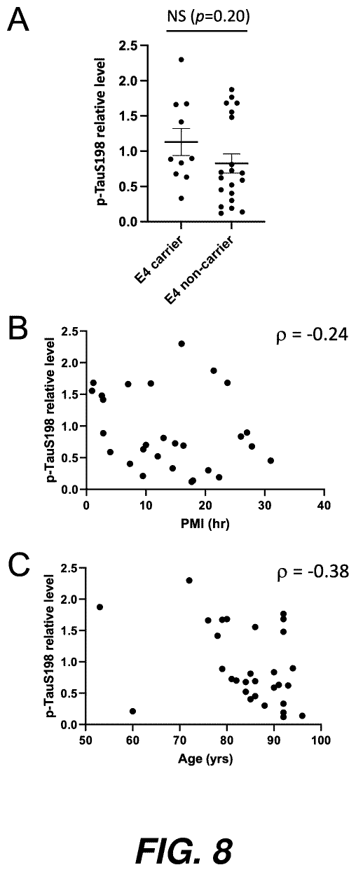

Brain p-tau198 levels significantly increased in MCI brains when compared with cognitively normal controls (1.18±0.15 vs. 0.75±0.11 relative units with a 1.57-fold increase and ap value <0.05; FIG. 7A). P-taul98 was capable of discriminating MCI cases from cognitively normal controls with an AUC value of 0.75 and CI_{95%}=0.58-0.92 (FIG. 7D). In comparison to two existing site-specific p-tau biomarkers T181 and T217, p-tau217 has somewhat better diagnostic performance with an AUC value of 0.85 and CI_{95%}=0.71-0.99 (FIGS. 7C and 7F). P-tau181 levels in MCI brains were not significantly elevated in comparison with those of cognitively normal brains (p=0.07; FIG. 7B) and diagnostic ROC curve showed lower discrimination capabilities (AUC=0.68; FIG. 7E). No significant difference was found between APOE4 carriers vs. non-carriers in relation to p-tau198 ELISA levels (FIG. 8A). Correlation analyses showed that there is no significant correlation between PMI vs. p-tau198 levels or age vs. p-tau levels measured by ELISA assays (FIGS. 8B and 8C).

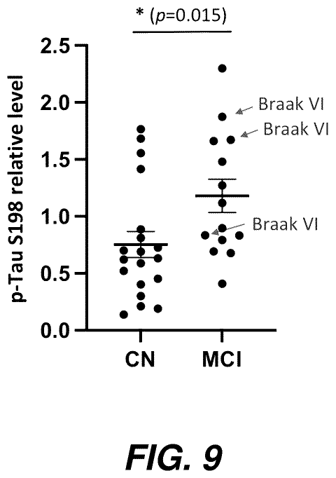

In summary, we demonstrated an experimental approach based on misfolded tau aggregation detection for new biomarker discovery. Using site-specific phospho-tau antibodies, such detection is therefore residue specific. We were able to identify multiple potential p-tau biomarkers from an antibody screening effort. Identified p-tau198 was able to not only diagnose AD from non-AD controls and AD from 4R-dominant PSP and CBD subjects with excellent differentiating power, more importantly, we showed p-tau198 is capable to discriminate MCI from cognitively normal subjects (FIG. 9).

### DISCUSSION

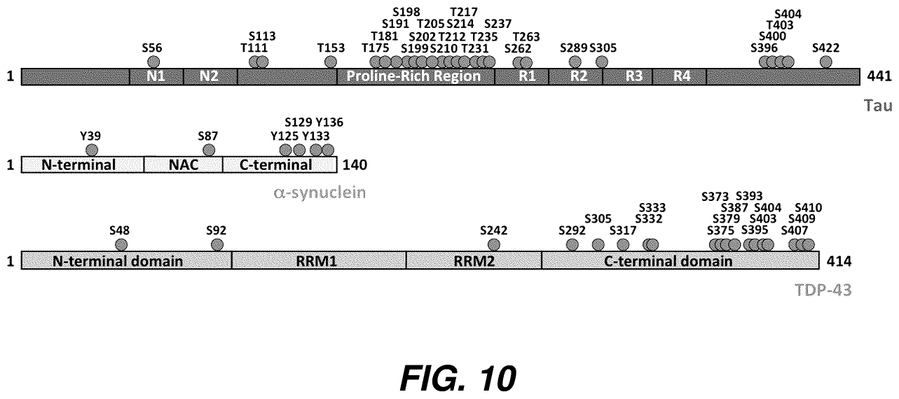

Posttranslational modifications such as tau hyperphosphorylation have been found in postmortem AD brains and may have significant implications for disease pathogenesis and progression. PTMs of tau proteins lead to structural and molecular diversity that may be linked to disease staging, and contribute to AD clinical heterogeneity and disease progression.^{15,16,26-28} High-molecular-weight oligomers of tau (HMW-tau) in postmortem AD brains have been reported in two recent reports.^{29,30} However, there have been no reports of systematic screening of using these AD-selective, HMW-tau aggregates as potential targets for AD diagnosis biomarker discovery. Our work independently discovered AD-specific, HMW-tau oligomers and we further identified several site-specific phospho-tau-detectable HMW-tau aggregates as potential AD biomarkers. Given that hyperphosphorylation is a common theme of PTM implicated in disease pathogenesis and progression in multiple amyloidogenic proteins including tau, α-synuclein, and TDP-43 in neurodegenerative diseases (FIG. 10),^{15-16-18,31-34} we envision our approach of biomarker discovery may be readily extended more broadly to other neurodegenerative disease diagnoses and translational detection applications.

Many PTMs sites in tau protein have been identified, which include dozens of phosphorylation sites, and over a dozen each of acetylation sites and ubiquitination sites.¹⁵ Based on the sequential accumulation model, different tau PTMs may occur at different AD stages and specific combinations of PTMs were proposed to reflect progressive steps in the process of tau fibril formation and AD disease progression. Therefore, Tau PTMs sites provided opportunities for antibody-based site-specific biomarker discovery, including biomarkers for early disease stages, which are urgently needed in clinical practice. Several site-specific p-tau biomarkers have been described and developed recently, including reported p-tau181 and p-tau217.^{13,21-24} As expected, our western blotting analysis and ELISA results confirmed reported p-tau181 and p-tau217 biomarkers. However, so far there are no well-established biomarkers that are capable to robustly discriminate MCI (or the prodromal stage of AD) from cognitively normal samples. Two p-tau biomarkers showed some potential for differentiating Aβ⁺ MCI cohorts from Aβ cognitively normal controls. Plasma p-tau181 levels were found to be elevated in Aβ⁺ cognitively unimpaired, Aβ⁺ MCI and Aβ⁺ AD dementia than in Aβ cognitively unimpaired and non-AD disease groups.¹³ CSF p-tau235 levels were elevated in amyloid-positive MCI cases from amyloid-negative cognitively unimpaired cohorts.35 Therefore, identification of p-tau198 or other promising candidates for general MCI diagnosis may have significant impact to clinical practice for much needed early AD biomarker development. Because of relatively small numbers of our cohorts, it will be interesting and important to further develop such biomarkers with significantly large case number of clinical cohorts and with AD patient plasma samples in the future incorporating ultrasensitive detection methods such as Single Molecular Array (Simoa) or Meso Scale Discovery Multi-Array technologies (MSD).³⁶⁻³⁷ It will also be interesting to compare the sensitivity of different ultrasensitive detection methods, for examples, misfolded protein-based amplification RT-QuIC technology³⁸⁻⁴⁰ versus p-tau aggregation-based Simoa tests.

Posttranslational modifications of hallmark amyloidogenic proteins in neurodegenerative diseases include but are not limited to phosphorylation. There are other observed PTMs, however, it is not fully clear what roles these PTMs play in various neurodegenerative diseases, including AD. It is conceivable that other PTM sites can also be utilized for developing site-specific biomarkers for disease diagnosis, staging, and differentiation in a similar fashion as phospho-tau biomarkers.

TABLE 1
Demographic characteristics of controls and MCI patients

| No. Case | Age | Sex | PMI | NeuroStatus | Neuropathological Diagnosis |
|---|---|---|---|---|---|
| 1 | 86 | M | 1 | CN | A1 (Thal 1), B1 (Braak I), C1; DLB McKetih brainstem |
| 2 | 80 | F | 1 | CN | A1 (Thal 1), B0, C0; Mild amyloid angiopathy; |
| | | | | | Mild atherosclerosis |
| 3 | 60 | M | 10 | CN | A1 (Thal 1), B1(Braak I), C0 |
| 4 | 78 | F | 3 | CN | A3 (Thal 4), B1 (Braak II), C1; Mild amyloid angiopathy |
| 5 | 88 | F | 20 | CN | A0, B1 (Braak I), C0; Mild amyloid angiopathy; |
| | | | | | Mild atherosclerosis |
| 6 | >89 | M | 4 | CN | A1 (Thal 2), B1 (Braak II), C1; Mild amyloid angiopathy; |
| | | | | | Moderate atherosclerosis; Moderate arteriolosclerosis |
| 7 | 85 | F | 13 | CN | A0, B1 (Braak II), C0; Mild atherosclerosis |
| 8 | 86 | M | 16 | CN | A1 (Thal 1), B1 (Braak I), C0; Mild atherosclerosis |
| 9 | 81 | F | 15 | CN | A1 (Thal 1), B1 (Braak II), C0; Moderate atherosclerosis |
| 10 | 82 | M | 10 | CN | A0, B1 (Braak II), C0; Hippocampal sclerosis |
| 11 | 85 | F | 7 | CN | A3 (Thal 5), B2 (Braak III), C1; Metastatic neoplasm; |
| | | | | | Mild arteriolosclerosis |
| 12 | >89 | M | 18 | CN | A0, B2 (Braak III), C0; Severe atherosclerosis; |
| | | | | | ARTAG; Hemorrhage |
| 13 | >89 | M | 22 | CN | A1 (Thal 2), B2 (Braak III), C0; ARTAG |
| 14 | 86 | M | 31 | CN | A3 (Thal 4), B2 (Braak III), C1; Severe amyloid angiopathy; |
| | | | | | Moderate atherosclerosis |
| 15 | >89 | F | unknown | CN | A1 (Thal 1), B2 (Braak III), C2; Mild amyloid angiopathy; Infarcts |
| 16 | >89 | F | 10 | CN | A3 (Thal 4), B2 (Braak III), C1; Severe arteriolosclerosis; |
| | | | | | ARTAG; Mild amyloid angiopathy |
| 17 | >89 | F | 24 | CN | A1 (Thal 1), B2 (Braak III), C0; Moderate arteriolosclerosis; |
| | | | | | Hypoxic-ischemic encephalopathy |
| 18 | 79 | F | 3 | CN | A3 (Thal 4), B2 (Braak IV), C2; Mild arteriolosclerosis |
| 19 | 84 | F | 12 | CN | A2 (Thal 3), B2 (Braak IV), C2; Mild atherosclerosis; Infarcts |
| 20 | >89 | M | unknown | CN | A3 (Thal 4), B2 (Braak IV), C1; Mild atherosclerosis; |
| | | | | | Moderate arteriolosclerosis; Mild amyloid angiopathy |
| 21 | >89 | M | 15 | MCI | A3 (Thal 4), B2 (Braak III), C1; Severe atherosclerosis; |
| | | | | | Moderate arteriolosclerosis; Moderate amyloid angiopathy; |
| | | | | | Microinfarct; ARTAG; LATE-NC |
| 22 | >89 | F | 10 | MCI | A2 (Thal 3), B2 (Braak III), C1; Severe arteriolosclerosis |
| 23 | >89 | F | 12.5 | MCI | A3 (Thal 5), B2 (Braak III), C1; DLB McKeith limbic; |
| | | | | | Moderate amyloid angiopathy; Moderate atherosclerosis; |
| | | | | | Severe arteriolosclerosis; Mild ARTAG |
| 24 | 81 | M | 7.2 | MCI | A3 (Thal 4), B2 (Braak III), C2; Infarcts; Harmartoma |
| 25 | 76 | F | 5 | MCI | A2 (Thal 3), B1 (Braak II), C2; Mild arteriolosclerosis |
| 26 | >89 | F | 3.4 | MCI | A2 (Thal 3), B2 (Braak III), C1; Moderate atherosclerosis |
| 27 | >89 | F | 8.8 | MCI | A2 (Thal 3), B2 (Braak III), C2; Mild amyloid angiopathy; |
| | | | | | Mild arteriolosclerosis |
| 28 | >89 | F | 16 | MCI | A2 (Thal 3), B2 (Braak IV), C2; Mild atherosclerosis |
| 29 | 84 | M | 17 | MCI | A3 (Thal 4), B2 (Braak IV), C2; DLB McKeith brainstem; |
| | | | | | Mild amyloid angiopathy; Hippocampal sclerosis; ARTAG |
| 30 | >89 | F | 3 | MCI | A3 (Thal 4), B2 (Braak IV), C1; Mild atherosclerosis; |
| | | | | | Moderate arteriolosclerosis; Moderate amyloid |
| | | | | | angiopathy; Contusion |
| 31 | 72 | F | 16 | MCI | A3 (Thal 4), B2 (Braak IV), C2; Mild amyloid |
| | | | | | angiopathy; Mild atherosclerosis |
| 32 | 76 | M | 16 | MCI | A3 (Thal 5), B3 (Braak V), C3; Severe amyloid |
| | | | | | angiopathy; Mild atherosclerosis; Contusion |
| 33 | 53 | M | 16 | MCI | A3 (Thal 5), B3 (Braak VI), C3; Moderate amyloid |
| | | | | | angiopathy; Severe amyloid angiopathy |
| 34 | >89 | F | 16 | MCI | A3 (Thal 5), B3 (Braak VI), C2; Severe amyloid |
| | | | | | angiopathy; Moderate arteriolosclerosis |
| 35 | 79 | M | 16 | MCI | A3 (Thal 4), B3 (Braak VI), C3; Severe amyloid |
| | | | | | angiopathy; Moderate arteriolosclerosis; Mild |
| | | | | | atherosclerosis; ARTAG; Microinfarct |

TABLE 2
Demographic characteristics of AD dementia and rare tauopathy patients

| No. Case | Age | Sex | PMI | NeuroStatus | Neuropathological Diagnosis |
|---|---|---|---|---|---|
| 36 | >89 | M | 16 | Dementia | A3 (Thal 5), B2 (Braak IV), C1; Severe amyloid angiopathy; |
| | | | | | Severe atherosclerosis; Moderate arteriolosclerosis; |
| | | | | | Infarcts; Hippocampal sclerosis |
| 37 | 88 | F | 16 | Dementia | A3 (That 4), B2 (Braak IV), C1; Mild amyloid angiopathy; |
| | | | | | Severe arteriolosclerosis |
| 38 | >89 | F | 12 | Dementia | A3 (Thal 4), B3 (Braak V), C2; ARTAG |
| 39 | 88 | F | 8 | Dementia | A3 (Thal 4), B3 (Braak V), C3; DLB McKeith neocortical; |
| | | | | | Moderate arteriolosclerosis; Infarcts; LATE-NC |
| 40 | 73 | F | 4 | Dementia | A3 (Thal 4), B3 (Braak V), C2; DLB McKeith neocortical; |
| | | | | | Mild amyloid angiopathy, Moderate arteriolosclerosis; |
| | | | | | LATE-NC |
| 41 | >89 | F | 16 | Dementia | A3 (Thal 5), B3 (Braak V), C2; DLB McKeith amygdala; |
| | | | | | Mild amyloid angiopathy; Severe atherosclerosis; |
| | | | | | Severe arteriolosclerosis; Microinfarct |
| 42 | 86 | F | 13 | Dementia | A3 (That 4), B3 (Braak V), C2; DLB McKeith neocortical; |
| | | | | | Mild amyloid angiopathy; Moderate arteriolosclerosis; |
| | | | | | Infarcts |
| 43 | 66 | M | 22 | Dementia | A3 (Thal 5), B3 (Braak V), C1; DLB McKeith neocortical |
| 44 | >89 | M | 14 | Dementia | A3 (Thal 5), B3 (Braak VI), C2; Moderate amyloid |
| | | | | | angiopathy, Moderate atherosclerosis |
| 45 | 69 | M | unknown | Dementia | A3 (Thal 4), B3 (Braak VI), C2; Lacunes |
| 46 | 63 | M | 2 | Dementia | A3 (Thal 4), B3 (Braak VI), C3; Severe amyloid angiopathy |
| 47 | 70 | M | 5 | Dementia | A3 (Thal 4), B3 (Braak VI), C2; DLB McKeith brainstem; |
| | | | | | Mild amyloid angiopathy; ARTAG |
| 48 | 55 | F | 10 | Dementia | A3 (Thal 5), B3 (Braak VI), C3; DLB McKeith amygdala; |
| | | | | | Severe amyloid angiopathy; Moderate arteriolosclerosis |
| 49 | 85 | F | 26 | Dementia | A3 (Thal 4), B3 (Braak VI), C3; DLB McKeith limbic; |
| | | | | | Severe amyloid angiopathy; Severe atherosclerosis |
| 50 | 83 | F | 4 | Dementia | A3 (Thal 4), B3 (Braak VI), C1; DLB McKeith neocortical; |
| | | | | | Mild arteriolosclerosis; Hippocampal sclerosis; |
| | | | | | ARTAG; LATE-NC |
| 51 | 69 | M | unknown | Dementia | A3 (Thal 5), B3 (Braak VI), C3; DLB McKeith neocortical; |
| | | | | | Mild amyloid angiopathy; Mild arteriolosclerosis; |
| | | | | | LATE-NC; Hippocampal sclerosis |
| 52 | >89 | F | 52 | Dementia | A3 (Thal 5), B3 (Braak VI), C2; DLB McKeith limbic; |
| | | | | | Moderate amyloid angiopathy; Mild atherosclerosis; |
| | | | | | Moderate arteriolosclerosis; Hippocampal sclerosis |
| 53 | 70 | M | 3 | Dementia | Braak V; AD; PSP; Mild atherosclerosis |
| 54 | 80 | F | 16 | Dementia | Pick's |
| 55 | 64 | F | 2 | Dementia | Pick's |
| 56 | 75 | F | 8 | Unknown | Pick's; Mild atherosclerosis |
| 57 | 81 | F | 13 | Dementia | Pick's; Moderate atheroslcerosis |
| 58 | 71 | F | 12 | Dementia | Pick's; Moderate atherosclerosis |
| 59 | 73 | F | 1 | Dementia | CBD; Mild atherosclerosis |
| 60 | 78 | F | 13 | Unknown | CBD; Mild amyloid angiopathy; Moderate atherosclerosis |
| 61 | 68 | F | 2 | Dementia | CBD |
| 62 | 75 | M | 12 | Dementia | CBD; Mild atherosclerosis |
| 63 | 68 | F | 24 | Dementia | CBD; Mild atherosclerosis; Lacunes |
| 64 | 57 | M | 17 | Dementia | CBD; Mild arteriolosclerosis |
| 65 | 72 | F | 16 | Dementia | CBD; Mild atheroclerosis; Severe amyloid angiopathy |
| 66 | 53 | M | 44 | Dementia | CBD; Mild arteriolosclerosis |
| 67 | >89 | F | 18 | Dementia | CBD; Moderate amyloid angiopathy; Moderate arteriolosclerosis |
| 68 | 61 | M | unknown | Unknown | PSP; Infarcts |
| 69 | 75 | F | 16 | Dementia | PSP |
| 70 | 78 | F | 2 | Dementia | PSP; Probable AD; Infarcts |
| 71 | 84 | F | 11 | Dementia | PSP; Possible AD; Mild atherosclerosis |
| 72 | 67 | M | 18 | Dementia | PSP |
| 73 | 82 | F | 25 | Dementia | PSP; Mild amyloid angiopathy; Mild atherosclerosis |
| 74 | >89 | M | 20 | Dementia | PSP; Severe atherosclerosis; Moderate arteriolosclerosis; |
| | | | | | Hippocampal sclerosis; LATE-NC |
| 75 | 86 | M | 19 | MCI | PSP; Mild atherosclerosis; Hippocampal sclerosis; ARTAG |
| 76 | >89 | F | 56 | CN | PSP; DLB McKeith limbic; Microinfract; Moderate |
| | | | | | atherosclerosis; Moderate arteriolosclerosis; ARTAG |

### REFERENCES FOR EXAMPLE 2

1. Braak, H.; Braak, E. Neuropathological staging of Alzheimer-related changes. Acta Neuropathol. 1991, 82, 239-259.
2. Selkoe, D. J. Alzheimer's disease: genes, proteins, and therapy. Physiol Rev. 2001, 81, 741-766.
3. Bloom, G. S. Amyloid-β and tau: the trigger and bullet in Alzheimer disease pathogenesis. JAMA Neurol. 2014, 71, 505-508.
4. Spires-Jones, T. L.; Hyman, B. T. The intersection of amyloid beta and tau at synapses in Alzheimer's disease. Neuron. 2014, 82, 756-771.
5. Wilcock, G. K.; Esiri, M. M. Plaques, tangles and dementia. A quantitative study. J Neurol Sci. 1982, 56, 343-356.
6. Serrano-Pozo, A.; Frosch, M. P.; Masliah, E.; Hyman, B. T. Neuropathological alterations in Alzheimer disease. Cold Spring Harb Perspect Med. 2011, 1, a006189.
7. Nelson, P. T.; Alafuzoff, I.; Bigio, E. H.; Bouras, C.; Braak, H.; Cairns, N. J.; Castellani, R. J.; Crain, B. J.; Davies, P.; Del Tredici, K.; Duyckaerts, C.; Frosch, M. P.; Haroutunian, V.; Hof, P. R.; Hulette, C. M.; Hyman, B. T.; Iwatsubo, T.; Jellinger, K. A.; Jicha, G. A.; Kovari, E.; Kukull, W. A.; Leverenz, J. B.; Love, S.; Mackenzie, I. R.; Mann, D. M.; Masliah, E.; McKee, A. C.; Montine, T. J.; Morris, J. C.; Schneider, J. A.; Sonnen, J. A.; Thal, D. R.; Trojanowski, J. Q.; Troncoso, J. C.; Wisniewski, T.; Woltjer, R. L.; Beach, T. G. Correlation of Alzheimer disease neuropathologic changes with cognitive status: a review of the literature. J Neuropathol Exp Neurol. 2012, 71, 362-381.
8. Goedert, M.; Spillantini, M. G. Propagation of Tau aggregates. Mol Brain. 2017, 10, 18.
9. Lee, V M.; Goedert, M.; Trojanowski, J. Q. Neurodegenerative tauopathies. Annu Rev Neurosci. 2001, 24, 1121-1159.
10. Spillantini, M. G.; Goedert, M. Tau protein pathology in neurodegenerative diseases. Trends Neurosci. 1998, 21, 428-433.
11. Grundke-Iqbal, I.; Iqbal, K.; Tung, Y C.; Quinlan, M.; Wisniewski, H. M.; Binder, L. I. Abnormal phosphorylation of the microtubule-associated protein tau (tau) in Alzheimer cytoskeletal pathology. Proc Natl Acad Sci USA. 1986, 83, 4913-4917.
12. Blennow, K.; Dubois, B.; Fagan, A. M.; Lewczuk, P.; de Leon, M. J.; Hampel, H. Clinical utility of cerebrospinal fluid biomarkers in the diagnosis of early Alzheimer's disease. Alzheimers Dement. 2015, 11, 58-69.
13. Janelidze, S.; Mattsson, N.; Palmqvist, S.; Smith, R.; Beach, T. G.; Serrano, G. E.; Chai, X.; Proctor, N. K.; Eichenlaub, U.; Zetterberg, H.; Blennow, K.; Reiman, E. M.; Stomrud, E.; Dage, J. L.; Hansson, O. Plasma P-tau181 in Alzheimer's disease: relationship to other biomarkers, differential diagnosis, neuropathology and longitudinal progression to Alzheimer's dementia. Nat Med. 2020, 26, 379-386.
14. Ashton, N. J.; Pascoal, T. A.; Karikari, T. K.; Benedet, A. L.; Lantero-Rodriguez, J.; Brinkmalm, G.; Snellman, A.; Scholl, M.; Troakes, C.; Hye, A.; Gauthier, S.; Vanmechelen, E.; Zetterberg, H.; Rosa-Neto, P.; Blennow, K. Plasma p-tau231: a new biomarker for incipient Alzheimer's disease pathology. Acta Neuropathol. 2021, 141, 709-724.
15. Wesseling, H.; Mair, W.; Kumar, M.; Schlaffner, C. N.; Tang, S.; Beerepoot, P.; Fatou, B.; Guise, A. J.; Cheng, L.; Takeda, S.; Muntel, J.; Rotunno, M. S.; Dujardin, S.; Davies, P.; Kosik, K. S.; Miller, B. L.; Berretta, S.; Hedreen, J. C.; Grinberg, L. T.; Seeley, W. W.; Hyman, B. T.; Steen, H.; Steen, J. A. Tau PTM Profiles Identify Patient Heterogeneity and Stages of Alzheimer's Disease. Cell. 2020, 183, 1699-1713. e13.
16. Arakhamia, T.; Lee, C. E.; Carlomagno, Y; Duong, D. M.; Kundinger, S. R.; Wang, K.; Williams, D.; DeTure, M.; Dickson, D. W.; Cook, C. N.; Seyfried, N. T.; Petrucelli, L.; Fitzpatrick, A. W. P. Posttranslational Modifications Mediate the Structural Diversity of Tauopathy Strains. Cell. 2020, 180, 633-644.e12.
17. Schindler, S. E.; Karikari, T. K. Comorbidities confound Alzheimer's blood tests. Nat Med. 2022, 28, 1349-1351.
18. Fitzpatrick, A. W. P.; Falcon, B.; He, S.; Murzin, A. G.; Murshudov, G.; Garringer, H. J.; Crowther, R. A.; Ghetti, B.; Goedert, M.; Scheres, S. H. W. Cryo-EM structures of tau filaments from Alzheimer's disease. Nature. 2017, 547, 185-190.
19. Falcon, B.; Zhang, W.; Murzin, A. G.; Murshudov, G.; Garringer, H. J.; Vidal, R.; Crowther, R. A.; Ghetti, B.; Scheres, S. H. W.; Goedert, M. Structures of filaments from Pick's disease reveal a novel tau protein fold. Nature. 2018, 561, 137-140.
20. Zhang, W.; Tarutani, A.; Newell, K. L.; Murzin, A. G.; Matsubara, T.; Falcon, B.; Vidal, R.; Garringer, H. J.; Shi, Y; Ikeuchi, T.; Murayama, S.; Ghetti, B.; Hasegawa, M.; Goedert, M.; Scheres, S. H. W. Novel tau filament fold in corticobasal degeneration. Nature. 2020, 580, 283-287.
21. Suarez-Calvet, M.; Karikari, T. K.; Ashton, N. J.; Lantero Rodriguez, J.; Milà-Alomà, M.; Gispert, J. D.; Salvadó, G.; Minguillon, C.; Fauria, K.; Shekari, M.; Grau-Rivera, O.; Arenaza-Urquijo, E. M.; Sala-Vila, A.; Sanchez-Benavides, G.; González-de-Echávarri, J. M.; Kollmorgen, G.; Stoops, E.; Vanmechelen, E.; Zetterberg, H.; Blennow, K.; Molinuevo, J. L.; ALFA Study. Novel tau biomarkers phosphorylated at T181, T217 or T231 rise in the initial stages of the preclinical Alzheimer's continuum when only subtle changes in Aβ pathology are detected. EMBO Mol Med. 2020, 12, e12921.
22. Karikari, T. K.; Emeršič, A.; Vrillon, A.; Lantero-Rodriguez, J.; Ashton, N. J.; Kramberger, M. G.; Dumurgier, J.; Hourregue, C.; Čučnik, S.; Brinkmalm, G.; Rot, U.; Zetterberg, H.; Paquet, C.; Blennow, K. Head-to-head comparison of clinical performance of CSF phospho-tau T181 and T217 biomarkers for Alzheimer's disease diagnosis. Alzheimers Dement. 2021, 17, 755-767.
23. Thijssen, E. H.; La Joie, R.; Strom, A.; Fonseca, C.; Iaccarino, L.; Wolf, A.; Spina, S.; Allen, I. E.; Cobigo, Y; Heuer, H.; VandeVrede, L.; Proctor, N. K.; Lago, A. L.; Baker, S.; Sivasankaran, R.; Kieloch, A.; Kinhikar, A.; Yu, L.; Valentin, M. A.; Jeromin, A.; Zetterberg, H.; Hansson, O.; Mattsson-Carlgren, N.; Graham, D.; Blennow, K.; Kramer, J. H.; Grinberg, L. T.; Seeley, W. W.; Rosen, H.; Boeve, B. F.; Miller, B. L.; Teunissen, C. E.; Rabinovici, G. D.; Rojas, J. C.; Dage, J. L.; Boxer, A. L.; Advancing Research and Treatment for Frontotemporal Lobar Degeneration investigators. Plasma phosphorylated tau 217 and phosphorylated tau 181 as biomarkers in Alzheimer's disease and frontotemporal lobar degeneration: a retrospective diagnostic performance study. Lancet Neurol. 2021, 20. 739-752.
24. Leuzy, A.; Mattsson-Carlgren, N.; Palmqvist, S.; Janelidze, S.; Dage, J. L.; Hansson, O. Blood-based biomarkers for Alzheimer's disease. EMBO Mol Med. 2022, 14, e14408.
25. Ashton, N. J.; Benedet, A. L.; Pascoal, T. A.; Karikari, T. K.; Lantero-Rodriguez, J.; Brum, W. S.; Mathotaarachchi, S.; Therriault, J.; Savard, M.; Chamoun, M.; Stoops, E.; Francois, C.; Vanmechelen, E.; Gauthier, S.; Zimmer, E. R.; Zetterberg, H.; Blennow, K.; Rosa-Neto, P. Cerebrospinal fluid p-tau231 as an early indicator of emerging pathology in Alzheimer's disease. EBioMedicine. 2022, 76, 103836.
26. Dujardin, S.; Commins, C.; Lathuiliere, A.; Beerepoot, P.; Fernandes, A. R.; Kamath, T. V; De Los Santos, M. B.; Klickstein, N.; Corjuc, D. L.; Corjuc, B. T.; Dooley, P. M.; Viode, A.; Oakley, D. H.; Moore, B. D.; Mullin, K.; Jean-Gilles, D.; Clark, R.; Atchison, K.; Moore, R.; Chibnik, L. B.; Tanzi, R. E.; Frosch, M. P.; Serrano-Pozo, A.; Elwood, F.; Steen, J. A.; Kennedy, M. E.; Hyman, B. T. Tau molecular diversity contributes to clinical heterogeneity in Alzheimer's disease. Nat Med. 2020, 26, 1256-1263.
27. Ramesh, M.; Gopinath, P.; Govindaraju, T. Role of Post-translational Modifications in Alzheimer's Disease. Chembiochem. 2020, 21, 1052-1079.
28. Xia, Y; Prokop, S.; Giasson, B. I. “Don't Phos Over Tau”: recent developments in clinical biomarkers and therapies targeting tau phosphorylation in Alzheimer's disease and other tauopathies. Mol Neurodegener. 2021, 16, 37.
29. Zhou, Y; Shi, J.; Chu, D.; Hu, W.; Guan, Z.; Gong, C. X.; Iqbal, K.; Liu, F. Relevance of Phosphorylation and Truncation of Tau to the Etiopathogenesis of Alzheimer's Disease. Front Aging Neurosci. 2018, 10, 27.
30. Miao, J.; Shi, R.; Li, L.; Chen, F.; Zhou, Y; Tung, Y C.; Hu, W.; Gong, C. X.; Iqbal, K.; Liu, F. Pathological Tau From Alzheimer's Brain Induces Site-Specific Hyperphosphorylation and SDS- and Reducing Agent-Resistant Aggregation of Tau in vivo. Front Aging Neurosci. 2019, 11, 34.
31. Schmid, A. W.; Fauvet, B.; Moniatte, M.; Lashuel, H. A. Alphα-synuclein post-translational modifications as potential biomarkers for Parkinson disease and other synucleinopathies. Mol Cell Proteomics. 2013, 12, 3543-3558.
32. Manzanza, N. O.; Sedlackova, L.; Kalaria, R. N. Alpha-Synuclein Post-translational Modifications: Implications for Pathogenesis of Lewy Body Disorders. Front Aging Neurosci. 2021, 13, 690293.
33. François-Moutal, L.; Perez-Miller, S.; Scott, D. D.; Miranda, V. G.; Mollasalehi, N.; Khanna, M. Structural Insights Into TDP-43 and Effects of Post-translational Modifications. Front Mol Neurosci. 2019, 12, 301.
34. Azevedo, R.; Jacquemin, C.; Villain, N.; Fenaille, F.; Lamari, F.; Becher, F. Mass Spectrometry for Neurobiomarker Discovery: The Relevance of Post-Translational Modifications. Cells. 2022, 11, 1279.
35. Lantero-Rodriguez, J.; Snellman, A.; Benedet, A. L.; Milà-Alomà, M.; Camporesi, E.; Montoliu-Gaya, L.; Ashton, N. J.; Vrillon, A.; Karikari, T. K.; Gispert, J. D.; Salvadó, G.; Shekari, M.; Toomey, C. E.; Lashley, T. L.; Zetterberg, H.; Suárez-Calvet, M.; Brinkmalm, G.; Rosa Neto, P.; Blennow, K. P-tau235: a novel biomarker for staging preclinical Alzheimer's disease. EMBO Mol Med. 2021, 13, e15098.
36. Rissin, D. M.; Kan, C. W.; Campbell, T. G.; Howes, S. C.; Fournier, D. R.; Song, L.; Piech, T.; Patel, P. P.; Chang, L.; Rivnak, A. J.; Ferrell, E. P.; Randall, J. D.; Provuncher, G. K.; Walt, D. R.; Duffy, D. C. Single-molecule enzyme-linked immunosorbent assay detects serum proteins at subfemtomolar concentrations. Nat Biotechnol. 2010, 28, 595-599.
37. Yang, T.; O'Malley, T. T.; Kanmert, D.; Jerecic, J.; Zieske, L. R.; Zetterberg, H.; Hyman, B. T.; Walsh, D. M.; Selkoe, D. J. A highly sensitive novel immunoassay specifically detects low levels of soluble Aβ oligomers in human cerebrospinal fluid. Alzheimers Res Ther. 2015, 7, 14.
38. Wang, Z.; Manca, M.; Foutz, A.; Martinez, M. C.; Raymond, G. J.; Race, B.; Orru, C. D.; Yuan, J.; Shen, P.; Li, B.; Lang, Y; Dang, J.; Adornato, A.; Williams, K.; Maurer, N. R.; Gambetti, P.; Xu, B.; Surewicz, W.; Petersen, R. B.; Dong, X.; Appleby, B. S.; Caughey, B.; Cui, L.; Kong, Q.; Zou, W. Early Preclinical Detection of Prions in the Skin of Prion-Infected Animals. Nat. Commun. 2019, 10, 247.
39. Kraus, A.; Saijo, E.; Metrick, M. A. 2^{nd}; Newell, K.; Sigurdson, C. J.; Zanusso, G.; Ghetti, B.; Caughey, B. Seeding selectivity and ultrasensitive detection of tau aggregate conformers of Alzheimer disease. Acta Neuropathol. 2019, 137, 585-598.
40. Wu, L.; Wang, Z.; Lad, S.; Gilyazova, N.; Dougharty, D. T.; Marcus, M.; Henderson, F.; Ray, W. K.; Sieliak, S.; Li, J.; Helm, R. F.; Zhu, X.; Bloom, G. S.; Wang, S. J.; Zou, W. Q.; Xu, B. Selective detection of misfolded tau from postmortem Alzheimer's disease brains. Front Aging Neurosci. 2022, 14, 945875.
41. Hulette, C. M.; Welsh-Bohmer, K. A.; Murray, M. G.; Saunders, A. M.; Mash, D. C.; McIntyre, L. M. Neuropathological and neuropsychological changes in “normal” aging: evidence for preclinical Alzheimer disease in cognitively normal individuals. J Neuropathol Exp Neurol. 1998, 57, 1168-1174.
42. Hyman, B. T.; Phelps, C. H.; Beach, T. G.; Bigio, E. H.; Cairns, N. J.; Carrillo, M. C.; Dickson, D. W.; Duyckaerts, C.; Frosch, M. P.; Masliah, E.; Mirra, S. S.; Nelson, P. T.; Schneider, J. A.; Thal, D. R.; Thies, B.; Trojanowski, J. Q.; Vinters, H. V.; Montine, T. J. National Institute on Aging-Alzheimer's Association guidelines for the neuropathologic assessment of Alzheimer's disease. Alzheimers Dement. 2012, 8, 1-13.
43. McKeith, I. G.; Boeve, B. F.; Dickson, D. W.; Halliday, G.; Taylor, J. P.; Weintraub, D.; Aarsland, D.; Galvin, J.; Attems, J.; Ballard, C. G.; Bayston, A.; Beach, T. G.; Blanc, F.; Bohnen, N.; Bonanni, L.; Bras, J.; Brundin, P.; Burn, D.; Chen-Plotkin, A.; Duda, J. E.; El-Agnaf, O.; Feldman, H.; Ferman, T. J.; Ffytche, D.; Fujishiro, H.; Galasko, D.; Goldman, J. G.; Gomperts, S. N.; Graff-Radford, N. R.; Honig, L. S.; Iranzo, A.; Kantarci, K.; Kaufer, D.; Kukull, W.; Lee, V. M. Y; Leverenz, J. B.; Lewis, S.; Lippa, C.; Lunde, A.; Masellis, M.; Masliah, E.; McLean, P.; Mollenhauer, B.; Montine, T. J.; Moreno, E.; Mori, E.; Murray, M.; O'Brien, J. T.; Orimo, S.; Postuma, R. B.; Ramaswamy, S.; Ross, O. A.; Salmon, D. P.; Singleton, A.; Taylor, A.; Thomas, A.; Tiraboschi, P.; Toledo, J. B.; Trojanowski, J. Q.; Tsuang, D.; Walker, Z.; Yamada, M.; Kosaka, K. Diagnosis and management of dementia with Lewy bodies: Fourth consensus report of the DLB Consortium. Neurology. 2017, 89, 88-100.
44. Cairns, N. J.; Bigio, E. H.; Mackenzie, I. R.; Neumann, M.; Lee, V. M.; Hatanpaa, K. J.; White, C. L. 3^{rd}; Schneider, J. A.; Grinberg, L. T.; Halliday, G.; Duyckaerts, C.; Lowe, J. S.; Holm, I. E.; Tolnay, M.; Okamoto, K.; Yokoo, H.; Murayama, S.; Woulfe, J.; Munoz, D. G.; Dickson, D. W.; Ince, P. G.; Trojanowski, J. Q.; Mann, D. M.; Consortium for Frontotemporal Lobar Degeneration. Neuropathologic diagnostic and nosologic criteria for frontotemporal lobar degeneration: consensus of the Consortium for Frontotemporal Lobar Degeneration. Acta Neuropathol. 2007, 114, 5-22.
45. Nelson, P. T.; Dickson, D. W.; Trojanowski, J. Q.; Jack, C. R.; Boyle, P. A.; Arfanakis, K.; Rademakers, R.; Alafuzoff, I.; Attems, J.; Brayne, C.; Coyle-Gilchrist, I T. S.; Chui, H. C.; Fardo, D. W.; Flanagan, M. E.; Halliday, G.; Hokkanen, S. R. K.; Hunter, S.; Jicha, G. A.; Katsumata, Y; Kawas, C. H.; Keene, C. D.; Kovacs, G. G.; Kukull, W. A.; Levey, A. I.; Makkinejad, N.; Montine, T. J.; Murayama, S.; Murray, M. E.; Nag, S.; Rissman, R. A.; Seeley, W. W.; Sperling, R. A.; White, C. L. 3^{rd}; Yu, L.; Schneider, J. A. Limbic-predominant age-related TDP-43 encephalopathy (LATE): consensus working group report. Brain. 2019, 142, 1503-1527.

### Example 3

### Brain P-Tau396 as a Biomarker for Pathological Changes in Mild Cognitive Impairment of Alzheimer'S Disease

### Materials and Methods

Subjects. Participants were enrolled in the autopsy and brain donation program of the Joseph and Kathleen Price Bryan Alzheimer's Disease Research Center (ADRC), as previously described.^{18,19} Clinical diagnosis of dementia, MCI, and normal cognition was determined by annual consensus meetings based upon the clinical dementia rating (CDR) and contemporaneous NIA-AA criteria. After death, the brains were processed and banked according to published protocol.¹⁸ Immunohistochemistry for β-amyloid, tau, α-synuclein, and TDP-43 were performed as previously described. Neuropathological assessment of Alzheimer's Disease Neuropathological Change (ADNC), Dementia with Lewy Bodies (DLB), Primary Age-Related Tauopathy (PART), Aging-Related Tau Astrogliopathy (ARTAG), Limbic-predominant Age-Related TDP-43 Encephalopathy Neuropathological Change (LATE-NC), and Cerebral Amyloid Angiopathy (CAA) were performed according to published guidelines.² All participants gave informed consent prior to autopsy. All protocols and the use of postmortem brain tissues for research were approved by the North Carolina Central University and Duke University Institutional Review Boards (IRB).

Brain tissue handling. Fresh frozen brain tissues from the temporal lobe were dissected and homogenized at 10% (w/v) in lysis buffer (10 mM Tris pH 7.4, 150 mM NaCl, 0.5% Nonidet P-40, 0.5% deoxycholate, 5 mM EDTA) on ice with a Fisherbrand 150 hand-held homogenizer as previously described.¹⁷ The brain homogenates were further subjected to centrifugation at 1,000×g for 3 min at 4° C. and the supernatants were collected following a protocol commonly used to extract amyloidogenic protein aggregates from brain homogenates.^{27,28} The supernatants contain both detergent-soluble aggregates and detergent-insoluble aggregates. The supernatants were stored at −80° C. for western blotting and ELISA measurements.

Western blot analysis. Postmortem brain homogenates (10% w/v) were resuspended in 1× Laemmli sample buffer (without reducing agent and no heating step applied), loaded onto 4-12% Bis-Tris NuPAGE (Invitrogen, Waltham, MA). Proteins were transferred to Immobilon FL membrane (Millipore, Burlington, MA) for 30 min at 100 volts. The membranes were then blocked with LI-COR blocking buffer, and probed with the appropriate primary antibodies as previously described.^{17,29} P-tau T181 mouse monoclonal antibody (mAb) AT270 (cat #MN1050) were purchased from Invitrogen (Waltham, MA). P-tau S202 (cat #AP0765), p-tau T205 rabbit polyclonal antibody (pAb) (cat #AP0168), p-tau T217 rabbit pAb (cat #AP1233), p-tau T231 rabbit mAb (cat #AP0053), p-tau S262 rabbit pAb (cat #AP0397), p-tau S396 rabbit pAb (cat #AP1028), and p-tau S404 rabbit pAb (cat #AP0170) were obtained from ABclonal (Woburn, MA). All primary antibodies were diluted 1:1000. Fluorescent labeled secondary antibodies, IRDye 680LT donkey anti-mouse IgG (Cat #926-68022, LI-COR, Inc. Lincoln, NE) and IRDye 800CW Donkey anti-rabbit IgG (Cat #926-32213 LI-COR, Inc. Lincoln, NE) were diluted to 1:50,000. The immunoreactivity was visualized by Odyssey western blot detection (LICOR, Inc. Lincoln, NE). GAPDH or β-actin was used as loading controls.

Semi-quantification of western blotting of HMW “smear” area/intensity results was performed as previously described.¹⁷ The immunoreactivity was first visualized and then HMW phospho-tau aggregate band quantification was analyzed by densitometry using Odyssey Western blot detection software (LICOR, Inc., Lincoln, NE). During the quantification process, equal rectangle area was set for each sample for normalization. All the background-subtracted density values were normalized with loading control (GAPDH or β-actin). Western blotting exposure time was optimized to avoid signal oversaturation.

ELISA measurements. Protein concentrations of the brain samples were measured by BCA assays (Pierce BCA protein assay kit, Thermo Fisher Scientific). ELISA assays were performed as previously described.¹⁷ 20 μg of total protein per well was used for ELISA assays and detailed experimental procedures according to manufacturer's instructions. ELISA kits for p-tau T181 (Cat #KHO0631), p-tau T231 (Cat #KHB8051), and p-tau S396 (Cat #KHB7031) were purchased from Invitrogen (Waltham, MA) and p-tau T217 ELISA kit (Cat #59672) was procured from Cell Signaling (Danvers, MA). The colorimetric signal development time was optimized to fall in the linear dynamic range to avoid oversaturation. The absorbance was measured at OD450 by a SPECTRAmax plus plate reader (Molecular Devices, San Jose, CA).

Phospho-tau396 immunohistochemistry (IHC). IHC was performed on formalin fixed paraffin embedded (FFPE) sections of the hippocampus with transentorhinal cortex (TErC) and occipitotemporal cortex (OTC) and sections of the superior temporal cortex (STC). Sections were deparaffinized in xylene and washed in absolute ETOH and 95% ETOH. Endogenous peroxidase activity was blocked with 1.875% H₂O₂ in methanol for 8 min. Samples were then washed and hydrated in distilled H₂O. A second blocking step was applied by submerging the sections in a dish containing 200 mL of 5% w/v nonfat dry milk in 0.05 M Tris buffer, pH 7.6, for 20 min at room temperature. Phospho-tau-S396 rabbit mAb (ABclonal, Cat #AP1028) was applied to each slide at 1:500 and allowed to incubate for 45 min at 37-40° C. Secondary antibodies were applied using the Dako EnVision Dual Link System-HRP (Agilent Technologies, Cat #K406189-2) and incubated for 30 min at 37-40° C. The slides were then developed with Dako DAB solution (Agilent Technologies, Cat #K346811-2) for 5 min and counterstained with Fisherfinest Hematoxylin+(ThermoFisher, Cat #220-100) for 15-25 sec. The slides were dehydrated through graded alcohols, cleared in xylene, and coverslipped in Permount mounting medium.

Image analysis. Whole slide imaging of p-tau396-stained slides was performed using an Aperio GT-450 scanner. Image analysis was performed on Visiopharm's Oncotopix Discovery software (Visiopharm, Westminster, CO). Areas with p-tau396-positive and negative staining were manually annotated on the training data set, and the software was trained to identify p-tau-396-positive pixels using a linear Bayesian regression algorithm. Regions of interest (ROI), including the hippocampus CA4, CA3, CA2, CA1, subiculum, TErC, OTC, and superior temporal cortex (STC) were manually segmented on the testing data set, and the total number of p-tau396-positive pixels and total pixels in each ROI was automatically calculated. The tau burden was defined as the ratio of p-tau396-positive pixels over total pixels in each ROI.

Statistical analysis. All data (except the tau burden from image analysis) are presented as the mean±S.E.M and the differences were analyzed with unpaired Student's t test as implemented within GraphPad Prism software (version 9.0). P values <0.05 were considered significant. Relative quantification of p-tau396, p-tau181, p-tau217, and p-tau231 in AD patients and control (CTL) subjects (AD versus control) were compared using GraphPad Prism 9.0 software. The specificity and sensitivity of tau with the specific phosphorylated sites in AD versus CTL subjects, AD versus tauopathy subjects, or MCI versus cognitively normal subjects were determined based on the area under the curve (AUC) of receiver operating characteristics (ROC) analysis. The 95% confidence interval (CI_{95%}) of AUC was calculated with Wilson/Brown method. The tau burden from image analysis of cognitively normal and MCI brains were compared in logarithmic scale using box-whisker plots in GraphPad software. Each box-whisker plot shows levels for minimum (Q0), 25^{th} percentile (Q1), 50^{th} percentile (Q2 or median), 75^{th} percentile (Q3) and maximum (Q4).

### Results

Identification of epitope-specific new p-tau sites for AD diagnosis. Selection of tau phosphorylation sites were primarily based on the high frequencies of these tau phosphorylation sites in AD patients from recent high-resolution quantitative proteomics map of PTMs on tau isoforms extracted from postmortem AD brain tissues,¹³ as well as availability of high-quality site-specific phospho-tau antibodies. Only p-tau sites present in AD patients with significant frequency (patient frequency >10%) were chosen. More than twenty such p-tau sites were identified and over a dozen p-tau epitopes were selected for western blot screening of the postmortem brain homogenates for HMW, site-specific p-tau antibody-detectable aggregated tau species.¹⁷ These sites include p-tau181, p-tau217, and p-tau231, sites that have recently been developed as AD biomarkers for diagnosis and/or for tracking disease progression. Therefore, these sites served as validation and comparison controls.^{8,10, 11,30-32}

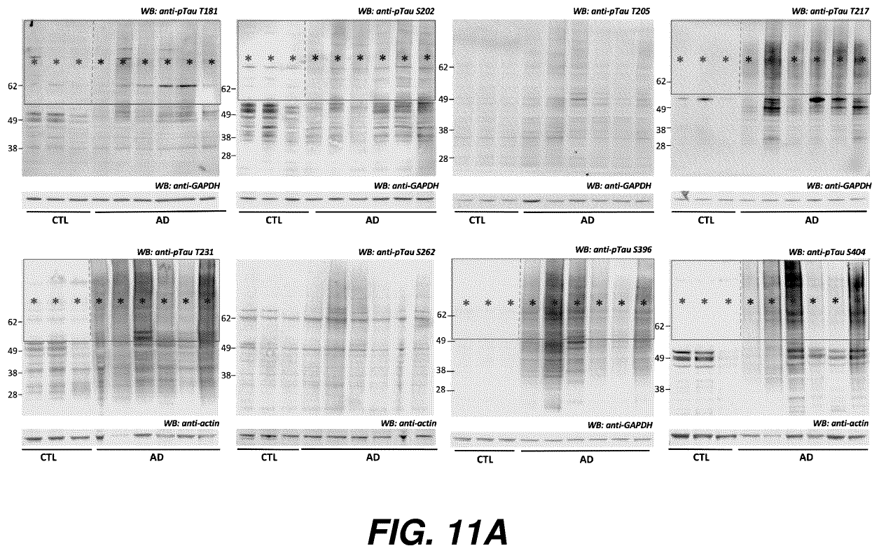

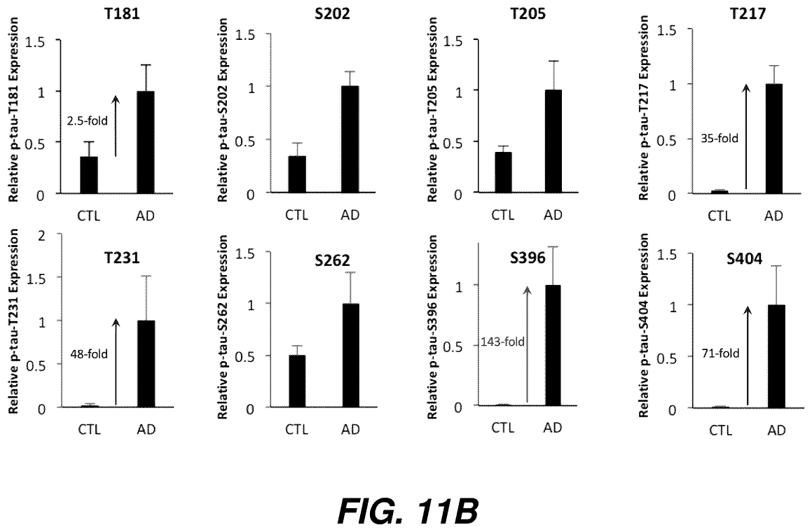

Similar to western blotting analyses with anti-tau antibodies,¹⁷ our search for candidate p-tau biomarkers was initially based on the differential intensities of HMW p-tau aggregate bands in the AD brains compared with normal controls. As expected, results from western blotting experiments with p-tau181, p-tau217, and p-tau231 (FIG. 11A) showed much stronger intensities of “smear-like” HMW protein aggregates from AD brains than those from control brains, detected by respective site-specific p-tau anti-bodies (highlighted by the rectangles with red or blue asterisks). Separate anti-GAPDH (or anti-β-actin) western analysis showed comparable amount of loading controls (FIG. 1A, lower panels). In addition to p-tau181, p-tau217, and p-tau231 controls, we also identified p-tau202, p-tau396, and p-tau404 antibodies that were able to differentiate AD brains from the control samples. In contrast, p-tau205 and p-tau262 sites were not able to discriminate AD from normal controls. Semi-quantification of the western blotting analysis of AD vs. control for the above p-tau antibodies is summarized in FIG. 11B. To our knowledge, p-tau202, p-tau396, and p-tau404 have not been systematically evaluated as potential biomarkers to differentiate AD from normal controls or MCI from cognitively normal brains.

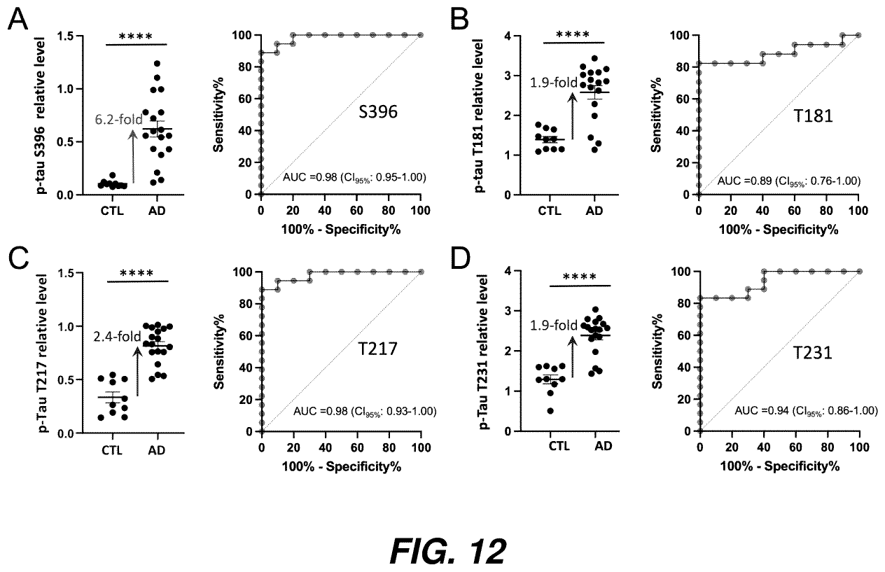

P-tau396 differentiates AD from normal controls. Identified from our antibody screening assays, p-tau396 is an epitope site with one of the biggest contrasts of phosphorylated tau aggregates between AD versus control brains (FIG. 11B). Mean values of HMW tau aggregates western blotting intensities were estimated to be 2.5-fold, 35-fold, 48-fold, 143-fold and 71-fold increase from control cases for p-tau181, p-tau217, p-tau231, p-tau396, and p-tau404 respectively. We therefore selected p-tau396 site for full characterization as a potential new biomarker for AD diagnosis. We quantified p-tau396 levels in the brain homogenates of temporal cortex in the discovery cohort using ELISA assays. Demographic characteristics and neuropathological diagnosis of this cohort of AD patients and non-AD control subjects are described in the Supplemental Table Sl. Brain p-tau396 levels were significantly increased in AD brains when compared with controls (0.62±0.08 vs. 0.10±0.01 relative units with a 6.2-fold increase and a p value of 2.7×10⁻⁶; FIG. 12, panel A). Diagnostic performance sensitivity and selectivity to identify AD cases from normal controls achieved perfect AUC=0.98 and CI_{95%}=0.95-1.00.

In comparison to the known p-tau epitope biomarkers T181, T217, and T231, p-tau396 showed similar or better differentiating powers than these three existing biomarkers to identify AD from controls. P-tau181 showed differentiating power with AUC=0.89 and CI_{95%}=0.76-1.00 (FIG. 12, panel B). Average p-tau181 concentrations were 2.58±0.17 relative units in the AD brains and 1.39±0.08 relative units in the control brains, a 1.9-fold increase with a p value of 2.48×10⁻⁶. P-tau217 showed differentiating power to identify AD from controls with AUC=0.98 and CI_{95%}=0.93-1.00 (FIG. 12, panel C). Average p-tau217 concentrations were 0.81±0.04 relative units in the AD brains and 0.33±0.05 relative units in the control brains, a 2.4-fold increase with a p value of 3.68×10⁻⁷. P-tau231 showed differentiating power to identify AD from controls with AUC=0.94 and CI_{95%}=0.86-1.00 (FIG. 12, panel D). Average p-tau231 concentrations were 2.39±0.11 relative units in the AD brains and 1.29±0.11 relative units in the control brains, a 1.9-fold increase with a p value of 3.86×10⁻⁷. Overall, p-tau396 detected a significantly higher ratio of p-tau concentration in AD brain over controls than those for p-tau181, p-tau217, and p-tau231, while differentiating diagnostic sensitivity and selectivity were comparable to those of p-tau217 but were better than those of p-tau181 and p-tau231 biomarkers (FIG. 12).

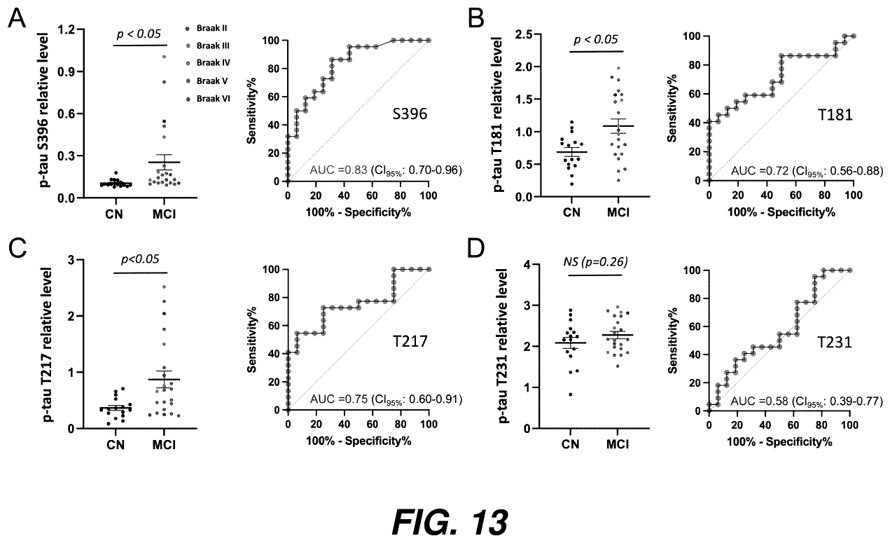

P-tau396 differentiates subjects with MCI from cognitively normal controls. To test the potential of p-tau396 as a biomarker for the more challenging MCI diagnosis, we quantified p-tau396 levels in the brain homogenates of temporal cortex in the discovery cohorts of MCI and cognitively normal subjects using ELISA assays. P-tau396 was capable of discriminating MCI brains from cognitively normal controls with an AUC value of 0.83 and CI₉₅%=0.70-0.96. Brain p-tau396 levels were significantly increased in MCI brains when compared with cognitively normal controls (0.254 vs. 0.104 relative units with a marked 2.43-fold increase and a p value <0.05; FIG. 13, panel A). P-tau biomarkers p-tau181 and p-tau217 also showed significantly elevated levels in MCI brains in comparison with cognitively normal brains, but both of them have significantly lower AUC values of 0.72 for p-tau181 and 0.75 for p-tau217 (FIG. 13, panels B and C, respectively). P-tau biomarker T231 did not show statistically significant elevation in p-tau levels in MCI compared to cognitively normal samples (p=0.26; FIG. 13, panel D). Correspondingly, ROC plot showed poor discriminating power in differentiating MCI brains from cognitively normal controls for p-tau231 (AUC=0.58). Therefore, our data (AUC values) suggest that p-tau396 displayed higher sensitivity and specificity than those of p-tau181, p-tau217, and p-tau231 in diagnosing MCI from cognitively normal brains. To the best of our knowledge, there are currently no well-established biomarkers to detect MCI.

We further analyzed our ELISA data of differentiating MCI from cognitively normal cases with regard to the Braak stages of the MCI cases and their contributions to differentiation for each of the four p-tau antibodies. In the scattered plots, we color-coded each MCI case based on Braak stages (FIG. 13). Interestingly, we found cases of high Braak stages (IV-VI) are the major contributors that enable differentiation between MCI from cognitively normal groups by both p-tau396 and p-tau217, but not by p-tau181 and p-tau231. This new finding revealed that p-tau396 (as well as p-tau217) may be more specifically targeted to tau neurofibrillary lesions that are consistent with Braak staging of the MCI cases.

We investigated if factors such as APOE4 genotype carrier, postmortem interval (PMI), and patient age at death may correlate with p-tau396 levels in MCI and cognitively normal brains. No significant difference in p-tau396 level was found between E4 carriers vs. non-carriers (p=0.22, student t-test; data not shown). Nonparametric Spearman's correlation analyses were performed on the other factors in relationship with brain p-tau396 levels. No strong correlation was found between PMI vs. p-tau396 levels (p=−0.0⁶), or age vs. p-tau396 levels (ρ=−0.34) (data not shown).

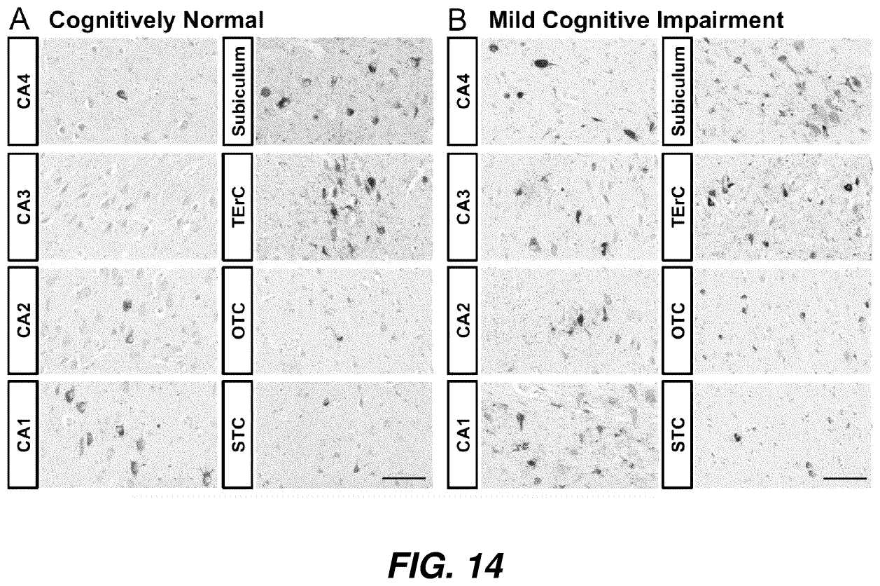

Phospho-tau396 IHC-derived tau burden correlates with antemortem cognitive status. To examine whether p-tau396 can be used to differentiate participants with MCI from those who are cognitively normal on postmortem neuropathological analysis, sections of the hippocampus and STC from a subset of our cohort were immunostained for p-tau396. Overall, p-tau396 showed a higher density of neurofibrillary tangles (NFT) in participants with MCI compared to cognitively normal controls (FIG. 14).

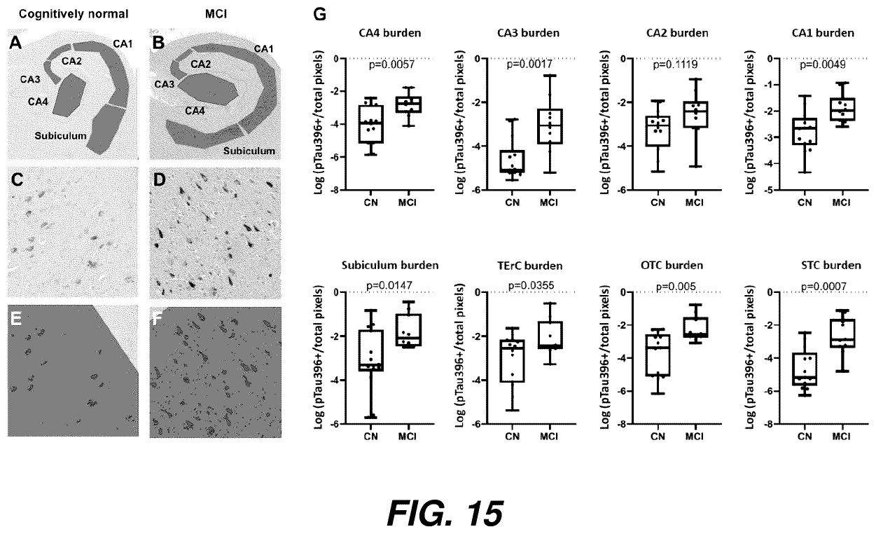

Recent studies have shown that artificial intelligence (AI)-derived neurofibrillary tangle burden was associated with antemortem cognitive impairment.³³ We applied A1 machine learning using the Visiopharm Oncotopix Discovery software to quantify p-tau396-positive pixels from whole slide images of p-tau396 IHC sections. Regions of interest (ROI), including the hippocampus CA4, CA3, CA2, CA1, subiculum, TErC, OTC, and STC were manually segmented, and the total number of p-tau396-positive pixels and total pixels in each ROI was automatically calculated after training with a linear Bayesian regression algorithm (FIG. 15, Panels A-F). Tau burden was defined as the ratio of p-tau396-positive pixels over total pixels of each ROL. Tau burden was significantly higher in the CA4, CA3, CA1, subiculum, TErC, OTC, and STC of participants with MCI (FIG. 5G). The difference in CA2 was not statistically significant.

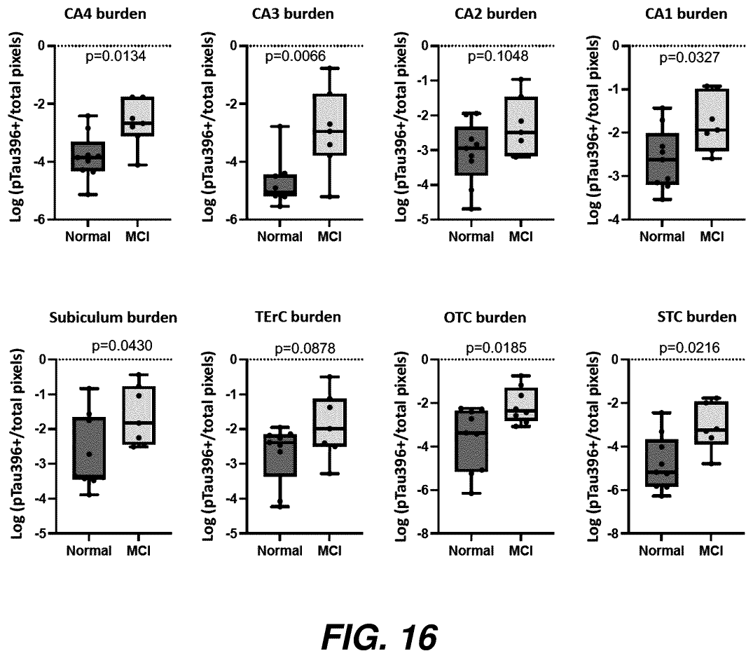

While Braak staging is the most widely used staging system to assess tau pathology in routine neuropathological diagnosis, individuals with normal cognition and MCI can have overlapping Braak stages.^{34,35} To test whether AI-derived tau burden based on p-tau396 IHC correlates with antemortem cognitive status in subjects with similar Braak stages, we repeated the above analysis on participants that are Braak stage III or IV only. Tau burden was significantly higher in the hippocampus CA4, CA3, CA1, subiculum, OTC, and STC of participants with MCI (FIG. 16). The differences in CA2 and TErC was not statistically significant.

### DISCUSSION

Phosphorylation at tau has been proposed as one of the earliest events in pathogenic tau formation.^{13,36} Several studies point to phosphorylations in the C-terminal of the tau molecule as being hallmark features of tauopathies, and phosphorylation on serine-396 is often designated as being of particular importance for disease progression of AD.³⁶⁻³⁹ However, to our knowledge, p-tau396 has not been fully characterized as an AD biomarker, in particular, for MCI detection. Moreover, the utility of p-tau396 for neuropathological tau staging and correlation with antemortem cognitive status has not been evaluated. Our work described here showed that brain p-tau396 displayed higher selectivity and sensitivity than those for p-tau181, p-tau217, and p-tau231 (FIG. 3). Consistent with this data, p-tau396 showed significantly higher fold of HMW tau aggregate intensities detected in western analyses (143-fold increase for p-tau396 vs. 2.5-fold for p-tau181, 35-fold for p-tau217, and 48-fold for p-tau231, FIG. 1B). Corroborative ELISA data in discriminating AD from controls showed p-tau396 achieved outstanding 6.2-fold increase of average p-tau396 values for AD group vs. control group, higher than 1.9-fold increase in both p-tau181 and p-tau231 detection and 2.4-fold increase in p-tau217 detection (FIG. 2).

So far there are no well-established biomarkers that are able to robustly discriminate MCI (or the prodromal stage of AD) from cognitively normal subjects. A few p-tau biomarkers showed some potential for differentiating Aβ+ MCI cohorts from Aβ− cognitively normal controls. Plasma p-tau181 levels were found to be elevated in A3+ cognitively unimpaired, A3+ MCI and Aβ+ AD dementia compared to Aβ− cognitively unimpaired and non-AD disease groups.¹⁰ Plasma p-tau217 levels were increased during the early preclinical stages of AD when insoluble tau aggregates were not yet detectable by tau-PET, therefore plasma p-tau217 also holds promise as a biomarker for early AD brain pathology that is consistent with our data.⁴⁰ CSF p-tau231 was sensitive to the earliest changes of Aβ in the medial orbitofrontal, precuneus and posterior cingulate before global Aβ PET positivity was reached.⁴¹ CSF p-tau235 levels were elevated in amyloid-positive MCI cases from amyloid-negative cognitively unimpaired cohorts.³¹ For challenging tasks of discriminating MCI from cognitively unimpaired controls, it is conceivable to use a panel of multiple biomarkers such as p-tau396 and p-tau217 to increase overall diagnostic power for future early AD diagnosis.

This study demonstrates a high-molecular-weight tau aggregation-based approach for novel histopathological biomarker discovery for AD diagnosis. Using site-specific phospho-tau antibodies, such detection is therefore residue specific. We were able to identify multiple potential p-tau biomarkers from such an antibody screening effort. An identified biomarker p-tau396 was highly capable of diagnosing AD from non-AD controls. Further, we showed that p-tau396 is capable of discriminating MCI from cognitively normal subjects. Moreover, using machine-learning based image analysis, we demonstrated that subjects with MCI showed higher p-tau396 burden in several regions of the hippocampus and temporal cortex compared to cognitively normal controls, even in subjects with comparable Braak stages, which is the most common staging scheme for tau burden in routine neuropathological diagnosis. These results indicate that brain p-tau396 levels strongly correlate with cognitive status during early stages of AD.

TABLE 3
Demographic characteristics of the samples.

| | CN | MCI | AD |
|---|---|---|---|
| | (N = 20) | (N = 16) | (N = 18) |
| Age, years | | | |
| Mean, (SD) | 85.3 | 85.3 | 79.3 |
| | (7.8) | (12.1) | (12.4) |
| Sex | | | |
| Female, n (%) | 11 | 10 | 10 |
| | (55.0%) | (62.5%) | (55.6%) |
| Male, n (%) | 9 | 6 | 8 |
| | (45.0%) | (37.5%) | (44.4%) |
| APOE ε4 status | | | |
| Non-carriers, n (%) | 17 | 7 | 8 |
| | (85.0%) | (43.8%) | (44.4%) |
| Carriers, n (%) | 3 | 9 | 8 |
| | (15.0%) | (56.2%) | (44.4%) |
| MMSE | | | |
| Mean, (SD) | 28.4 | 26 | 15 |
| | (1.8) | (2.6) | (6.6) |

Abbreviations: AD, Alzheimer's disease; APOE ε4, apolipoprotein epsilon 4; CN, cognitively normal; MCI, mild cognitive impairment; MMSE, Mini-Mental State Examination; SD, standard deviation.

### REFERENCES FOR EXAMPLE 3

1. Braak H, Braak E. Neuropathological staging of Alzheimer-related changes. Acta Neuropathol 1991; 82:239-259.
2. Selkoe D J. Alzheimer's disease: genes, proteins, and therapy. Physiol Rev 2001; 81:741-766.
3. Goedert M, Spillantini M G. Propagation of Tau aggregates. Mol Brain 2017; 10:18.
4. Lee V M, Goedert M, Trojanowski J Q. Neurodegenerative tauopathies. Annu Rev Neurosci 2001; 24:1121-1159.
5. Spillantini M G, Goedert M. Tau protein pathology in neurodegenerative diseases. Trends Neurosci 1998; 21:428-433.
6. Grundman M. Mild cognitive impairment can be distinguished from alzheimer disease and normal aging for clinical trials. Arch Neurol 2004; 61,59-66.
7. Ferman T J, Smith G E, Kantarci K, Boeve B F, et al. Nonamnestic mild cognitive impairment progresses to dementia with Lewy bodies. Neurology 2013; 81:2032-2038.
8. Ashton N J, Pascoal T A, Karikari T K, Benedet A L, et al. Plasma p-tau231: a new biomarker for incipient Alzheimer's disease pathology. Acta Neuropathol 2021; 141:709-724.
9. Blennow K, Dubois B, Fagan A M, Lewczuk P, et al. Clinical utility of cerebrospinal fluid biomarkers in the diagnosis of early Alzheimer's disease. Alzheimers Dement 2015; 11:58-69.
10. Janelidze S, Mattsson N, Palmqvist S, Smith R, et al. Plasma P-tau181 in Alzheimer's disease: relationship to other biomarkers, differential diagnosis, neuropathology and longitudinal progression to Alzheimer's dementia. Nat Med 2020; 26:379-386.
11. Thijssen E H, La Joie R, Strom A, Fonseca C, et al. Plasma phosphorylated tau 217 and phosphorylated tau 181 as biomarkers in Alzheimer's disease and frontotemporal lobar degeneration: a retrospective diagnostic performance study. Lancet Neurol 2021; 20:739-752.
12. Arakhamia T, Lee C E, Carlomagno Y, Duong D M, et al. Posttranslational Modifications Mediate the Structural Diversity of Tauopathy Strains. Cell 2020; 180:633-644.e12.
13. Wesseling H, Mair W, Kumar M, Schlaffner C N, et al. Tau PTM Profiles Identify Patient Heterogeneity and Stages of Alzheimer's Disease. Cell 2020; 183:1699-1713.e13.
14. Falcon B, Zhang W, Murzin A G, Murshudov G, et al. Structures of filaments from Pick's disease reveal a novel tau protein fold. Nature 2018; 561:137-140.
15. Fitzpatrick A W P, Falcon B, He S, Murzin A G, et al. Cryo-EM structures of tau filaments from Alzheimer's disease. Nature 2017; 547:185-190.
16. Zhang W, Tarutani A, Newell K L, Murzin A G, et al. Novel tau filament fold in corticobasal degeneration. Nature 2020; 580:283-287.
17. Wu L, Gilyazova N, Ervin J F, Wang S J, et al. Site-Specific Phospho-Tau Aggregation-Based Biomarker Discovery for AD Diagnosis and Differentiation. ACS Chem Neurosci 2022; 13:3281-3290.
18. Hulette C M, Welsh-Bohmer K A, Crain B, Szymanski M H, et al. Rapid brain autopsy. The Joseph and Kathleen Bryan Alzheimer's Disease Research Center experience. Arch Pathol Lab Med 1997; 121:615-618.
19. Hulette C M, Welsh-Bohmer K A, Murray M G, Saunders A M, et al. Neuropathological and neuropsychological changes in “normal” aging: evidence for preclinical Alzheimer disease in cognitively normal individuals. J Neuropathol Exp Neurol 1998; 57:1168-1174.
20. Wang S J, Guo Y, Ervin J F, Lusk J B, et al. Neuropathological associations of limbic-predominant age-related TDP-43 encephalopathy neuropathological change (LATE-NC) differ between the oldest-old and younger-old. Acta Neuropathol 2022; 144:45-57.
21. Crary J F, Trojanowski J Q, Schneider J A, Abisambra J F, Abner E L, Alafuzoff I, et al. (2014) Primary age-related tauopathy (PART): a common pathology associated with human aging. Acta Neuropathol. 128:755-766.
22. Hyman B T, Phelps C H, Beach T G, Bigio E H, et al. National Institute on Aging-Alzheimer's Association guidelines for the neuropathologic assessment of Alzheimer's disease. Alzheimers Dement 2012; 8:1-13.
23. Kovacs G G, Ferrer I, Grinberg L T, Alafuzoff I, et al. Aging-related tau astrogliopathy (ARTAG): harmonized evaluation strategy. Acta Neuropathol 2016; 131:87-10265.
24. McKeith I G, Boeve B F, Dickson D W, Halliday G, et al. Diagnosis and management of dementia with Lewy bodies: Fourth consensus report of the DLB Consortium. Neurology 2017; 89:88-100.
25. Nelson P T, Dickson D W, Trojanowski J Q, Jack C R, et al. Limbic-predominant age-related TDP-43 encephalopathy (LATE): consensus working group report. Brain 2019; 142:1503-1527.
26. Vonsattel J P, Myers R H, Hedley-Whyte E T, Ropper A H, et al. Cerebral amyloid angiopathy without and with cerebral hemorrhages: a comparative histological study. Ann Neurol 1991; 30:637-649.
27. Cali I, Castellani R, Yuan J, A1-Shekhlee A, et al. Classification of Sporadic Creutzfeldt-jakob Disease Revisited. Brain 2006; 129:2266-2277.
28. Dohler F, Sepulveda-Falla D, Krasemann S, Altmeppen H, et al. High molecular mass assemblies of amyloid-β oligomers bind prion protein in patients with Alzheimer's disease. Brain 2014; 137(Pt 3):873-886.
29. Wu L, Wang Z, Lad S, Gilyazova N, et al. Selective Detection of Misfolded Tau From Postmortem Alzheimer's Disease Brains. Front Aging Neurosci 2022; 14:945875.
30. Karikari T K, Emeršič A, Vrillon A, Lantero-Rodriguez J, et al. Head-to-head comparison of clinical performance of CSF phospho-tau T181 and T217 biomarkers for Alzheimer's disease diagnosis. Alzheimers Dement 2021; 17:755-767.
31. Lantero-Rodriguez J, Snellman A, Benedet A L, Milà-Alomà M, et al. P-tau235: a novel biomarker for staging preclinical Alzheimer's disease. EMBO Mol Med 2021; 13:e15098.
32. Suárez-Calvet M, Karikari T K, Ashton N J, Lantero Rodríguez J, et al. Novel tau biomarkers phosphorylated at T181, T217 or T231 rise in the initial stages of the preclinical Alzheimer's continuum when only subtle changes in Aβ pathology are detected. EMBO Mol Med 2020; 12:e12921.
33. Marx G A, Koenigsberg D G, McKenzie A T, Kauffman J, et al. Artificial intelligence-derived neurofibrillary tangle burden is associated with antemortem cognitive impairment. Acta Neuropathol Commun 2022; 10:157.
34. Mufson E J, Malek-Ahmadi M, Perez S E, Chen K. Braak staging, plaque pathology, and APOE status in elderly persons without cognitive impairment. Neurobiol Aging 2016; 37:147-153.
35. Petersen R C, Parisi J E, Dickson D W, Johnson K A, et al. Neuropathologic features of amnestic mild cognitive impairment. Arch Neurol 2006; 63:665-672.
36. Mondragón-Rodriguez S, Perry G, Luna-Munoz J, Acevedo-Aquino M C, et al. Phosphorylation of tau protein at sites Ser(396-404) is one of the earliest events in Alzheimer's disease and Down syndrome. Neuropathol Appl Neurobiol 2014; 40:121-135.
37. Helboe L, Rosenqvist N, Volbracht C, Pedersen, LØ, et al. Highly specific and sensitive target binding by the humanized pS396-Tau antibody hC10.2 across a wide spectrum of Alzheimer's disease and primary tauopathy postmortem brains. J. Alzheimer's Dis 2022; 88:207-228.
38. Rosenqvist N, Asuni A A, Andersson C R, Christensen S, et al. Highly specific and selective anti-pS396-tau antibody C10.2 targets seeding-competent tau. Alzheimer's Dementia: Translational Research & Clinical Interventions 2018; 4:521-534.
39. Hu Y Y, He S S, Wang X, Duan Q H, et al. Levels of nonphosphorylated and phosphorylated tau in cerebrospinal fluid of Alzheimer's disease patients: an ultrasensitive bienzyme-substrate-recycle enzyme-linked immunosorbent assay. Am J Pathol 2002; 160:1269-1278.
40. Janelidze S, Berron D, Smith R, Strandberg O, et al. Associations of plasma phospho-tau217 levels with tau positron emission tomography in early Alzheimer disease. JAMA Neurol 2021; 78:149-156.
41. Ashton N J, Benedet A L, Pascoal T A, Karikari T K, et al. Cerebrospinal fluid p-tau231 as an early indicator of emerging pathology in Alzheimer's disease. EBioMedicine 2022; 76:103836.
42. Rissin D M, Kan C W, Campbell T G, Howes S C, et al. Single-molecule enzyme-linked immunosorbent assay detects serum proteins at subfemtomolar concentrations. Nat Biotechnol 2010; 28:595-599.
43. Yang T, O'Malley T T, Kanmert D, Jerecic J, et al. A highly sensitive novel immunoassay specifically detects low levels of soluble Aβ oligomers in human cerebrospinal fluid. Alzheimer's Res Ther 2015:7:14.
44. Barthelémy N R, Horie K, Sato C, Bateman R J. Blood plasma phosphorylated-tau isoforms track CNS change in Alzheimer's disease. J Exp Med 2020; 217:e20200861.
45. Lam B, Masellis M, Freedman M, Stuss D T, et al. Clinical, imaging, and pathological heterogeneity of the Alzheimer's disease syndrome. Alzheimer's Res Ther 2013; 5:1.
46. Warren J D, Fletcher P D, Golden H L. The paradox of syndromic diversity in Alzheimer disease. Nat Rev Neurol 2012; 8:451-464.
47. Braak H, Del Tredici K. Evolutional aspects of Alzheimer's disease pathogenesis. J Alzheimers Dis 2013; 33:5155-161.
48. Irwin D J, Brettschneider J, McMillan C T, Cooper F, et al. Deep clinical and neuropathological phenotyping of Pick disease. Ann Neurol 2016; 79:272-287.
49. Williams D R, Holton J L, Strand C, Pittman A, et al. Pathological tau burden and distribution distinguishes progressive supranuclear palsy-parkinsonism from Richardson's syndrome. Brain 2007; 130:1566-1576.
50. Ling H, Kovacs G G, Vonsattel J P, Davey K, et al. Astrogliopathy predominates the earliest stage of corticobasal degeneration pathology. Brain 2016; 139:3237-3252.

### Example 4

### P-Tau422: A Novel Brain Biomarker for Detecting Mild Cognitive Impairment of Alzheimer'S Disease

### Methods

Brain tissue collection, handling, and analysis. Postmortem brain tissues from cognitively normal subjects, MCI, and AD dementia patients were collected from the Bryan Brain Bank and Biorepository of the Duke-UNC Alzheimer's Disease Research Center (Duke/UNC ADRC). Demographics, NeuroStatus, Braak staging of AD-tau and other neuropathology diagnosis are summarized in Table 3. All participants were enrolled in the autopsy and brain donation program of the Joseph and Kathleen Pryce Bryan ADRC as previously described.¹⁵ All subjects gave informed consent. All protocols and the use of postmortem brain tissues for research were approved by North Carolina Central University and Duke University Institutional Review Board (IRB). Neuropathological evaluation was performed following published guidelines.¹⁶⁻¹⁹ Brain tissues were homogenized at 10% (w/v) in lysis buffer (10 mM Tris pH 7.4, 150 mM NaCl, 0.5% Nonidet P-40, 0.5% deoxycholate, 5 mM EDTA) on ice with a Fisherbrand 150 hand-held homogenizer by a dedicated research staff for consistency. The brain homogenates were further subjected to centrifugation at 1,000×g for 3 min at 4° C. and the supernatants were collected and stored at −80° C. for Western blotting and ELISA (enzyme-linked immunosorbent assay) analyses.

Western blotting analysis. Brain homogenates (10% w/v) were resuspended in 1× Laemmli sample buffer (no reducing agent, no heating), loaded onto 4-12% Bis-Tris NuPAGE (Invitrogen, Waltham, MA). Proteins were transferred to Immobilon FL membrane (Millipore, Burlington, MA) for 30 min at 100 volts. The membranes were then blocked with LI-COR blocking buffer, and probed with the appropriate primary antibodies. P-Tau-Thr181 mAb AT270 (cat #MN1050, 1:1000) were purchased from Invitrogen (Waltham, MA). Tau Rabbit pAb A1103 (cat #AP1103, 1:1000 dilution), p-Tau S202(cat #AP0765, 1:1000 dilution), p-Tau T205 rabbit pAb, (cat #AP0168, dilution 1:1000), p-Tau T217 rabbit pAb (cat #AP1233, 1:1000 dilution), p-Tau T231 Rabbit mAb (cat #AP0053, 1:1000 dilution), p-Tau S262 Rabbit pAb (cat #AP0397, 1:1000 dilution), and p-Tau S404 Rabbit pAb (cat #AP0170, 1:1000 dilution) were obtained from ABclonal (Woburn, MA). P-tau S422 rabbit mAb (cat #ab79415, 1:1000 dilution) was purchased from abeam (Waltham, MA). Fluorescent labeled secondary antibodies, IRDye 680LT donkey anti-mouse IgG (cat #926-68022, LI-COR, Inc. Lincoln, NE) and IRDye 800CW Donkey anti-rabbit IgG (cat #926-32213 LI-COR, Inc. Lincoln, NE) were diluted to 1:50,000. The immunoreactivity was visualized by Odyssey Western blot detection (LICOR, Inc. Lincoln, NE). Western blotting exposure time was optimized to avoid signal oversaturation. GAPDH or β-actin was used as loading control. Control Western blotting analyses were performed the same as regular analyses except that no primary anti-bodies were applied to confirm antibody-specific detection as described previously.²⁰

ELISA measurements. Brain homogenates (10% w/v) were resuspended in 1× Laemmli sample buffer (no reducing agent, no heating), loaded onto 4-12% Bis-Tris NuPAGE (Invitrogen, Waltham, MA). Proteins were transferred to Immobilon FL membrane (Millipore, Burlington, MA) for 30 min at 100 volts. The membranes were then blocked with LI-COR blocking buffer, and probed with the appropriate primary antibodies. P-Tau-Thr181 mAb AT270 (cat #MN1050, 1:1000) were purchased from Invitrogen (Waltham, MA). Tau Rabbit pAb A1103 (cat #AP1103, 1:1000 dilution), p-Tau S202(cat #AP0765, 1:1000 dilution), p-Tau T205 rabbit pAb, (cat #AP0168, dilution 1:1000), p-Tau T217 rabbit pAb (cat #AP1233, 1:1000 dilution), p-Tau T231 Rabbit mAb (cat #AP0053, 1:1000 dilution), p-Tau S262 Rabbit pAb (cat #AP0397, 1:1000 dilution), and p-Tau S404 Rabbit pAb (cat #AP0170, 1:1000 dilution) were obtained from ABclonal (Woburn, MA). P-tau S422 rabbit mAb (cat #ab79415, 1:1000 dilution) was purchased from abeam (Waltham, MA). Fluorescent labeled secondary antibodies, IRDye 680LT donkey anti-mouse IgG (cat #926-68022, LI-COR, Inc. Lincoln, NE) and IRDye 800CW Donkey anti-rabbit IgG (cat #926-32213 LI-COR, Inc. Lincoln, NE) were diluted to 1:50,000. The immunoreactivity was visualized by Odyssey Western blot detection (LICOR, Inc. Lincoln, NE). Western blotting exposure time was optimized to avoid signal oversaturation. GAPDH or β-actin was used as loading control. Control Western blotting analyses were performed the same as regular analyses except that no primary anti-bodies were applied to confirm antibody-specific detection as described previously.²⁰

Phospho-tau422 immunochemistry (IHC). IHC was performed on formalin fixed paraffin embedded (FFPE) sections of the hippocampus with transentorhinal cortex (TErC) and occipitotemporal cortex (OTC) and sections of the superior temporal cortex (STC). Sections were deparaffinized in xylene and washed in absolute ETOH and 95% ETOH. Endogenous peroxidase activity was blocked with 1.875% H₂O₂ in methanol for 8 min. Samples were then washed and hydrated in DH2O. A second blocking step was applied by submerging the sections in a dish containing 200 mL of 5% w/v nonfat dry milk in 0.05 M Tris buffer, pH7.6, for 20 min at room temperature. Phospho-tau-S422 rabbit monoclonal antibody (abeam, Cat #ab79415) was applied to each slide at 1:500 and allowed to incubate for 45 min at 37-40° C. Secondary antibodies were applied using the Dako EnVision™ Dual Link System-HRP (Agilent Technologies, Cat #K406189-2) and incubated for 30 min at 37-40° C. The slides were then developed with Dako DAB solution (Agilent Technologies, Cat #K346811-2) for 5 min and counterstained with Fisherfinest Hematoxylin+(ThermoFisher, Cat #220-100) for 15-25 sec. The slides were dehydrated through graded alcohols, cleared in xylene, and coverslipped in Permount mounting medium.

Image analysis. Whole slide imaging of p-tau422-stained slides was performed using an Aperio AT-2 scanner. Image analysis was performed on Visiopharm's Oncotopix Discovery software (Visiopharm, Westminster, CO). Areas with p-tau422-positive staining were manually annotated, and the software was trained to identify p-tau-422-positive pixels using a linear Bayesian regression algorithm. Regions of interest (ROI), including the hippocampus CA4, CA3, CA2, CA1, subiculum, TErC, OTC, and STC were manually segmented, and the total number of p-tau422-positive pixels and total pixels in each ROI was automatically calculated. The tau burden was defined as the ratio of p-tau422-positive pixels over total pixels in each ROI.

Statistical analysis. All data are presented as the mean±S.E.M and the differences were analyzed with unpaired Student's t test as implemented within GraphPad Prism software (version 9.0). p values <0.05 were considered significant. Relative quantification of p-tau S422, T181, T217, and T231 in AD patients and control subjects (AD vs. CTL) were compared using GraphPad Prism 9.0 software. The specificity and sensitivity of tau with the specific phosphorylated sites in AD vs. CTL subjects, or MCI vs. cognitively normal subjects were determined based on the area under the curve (AUC) of receiver operating characteristics (ROC) analysis. The 95% confidence interval (CI95%) of AUC was calculated with Wilson/Brown method. The tau burden from image analysis of cognitively normal and MCI brains were compared in logarithmic scale using box-whisker plots in GraphPad software. Each box-whisker plot shows levels for minimum (Q0), 25th percentile (Q1), 50th percentile (Q2 or median),₇₅₁ percentile (Q3) and maximum (Q4).

### Results

Identification of epitope-specific novel phospho-tau sites for AD diagnosis. Selection of site-specific p-tau antibodies were primarily based on the high frequencies of these tau phosphorylation sites in AD patients from recent high-resolution quantitative proteomic mapping of PTMs on tau isoforms extracted from postmortem AD brain tissues,⁸ as well as available site-specific quality tau antibodies. Only p-tau sites with significant AD patient frequencies were chosen (patient frequency >10%). More than two dozen such epitopes were identified and over a dozen p-tau sites were selected for screening. Screening assays primarily utilized the postmortem brain homogenates for high-molecular-weight (HMW), site-specific p-tau antibody-detectable aggregated tau species with western blotting analysis. The epitopes chosen included p-tau181, p-tau217, and p-tau231 sites that were recently being developed as AD biomarkers for diagnosis and/or for disease staging, these sites served as validation controls.^{8-10,21-23}

Similar to western blotting analyses with anti-tau antibodies [27], our searches for candidate p-tau biomarkers were initially based on qualitative evaluation of presence/absence of the HMW p-tau aggregate bands in the comparison of AD brains with normal controls. As expected, p-tau181, p-tau217, and p-tau231 blots showed the presence of much stronger intensities of “smear-like” HMW protein aggregates from AD brains than those from control brains, detected by respective site-specific p-tau anti-bodies (highlighted by the rectangles with red or blue asterisks). Separate anti-GAPDH (or anti-β-actin) western analysis showed an approximate comparable amount of loading controls. In addition to p-tau181, p-tau217, and p-tau231 controls, we identified p-tau202, p-tau404, and p-tau422 sites that differentiated AD brains from the control samples. However, p-tau205 and p-tau262 sites were not able to discriminate AD from normal controls. To our knowledge, p-tau202, p-tau404, and p-tau422 have not been systematically evaluated as potential biomarkers to differentiate AD from normal controls. We further compared p-tau HMW aggregate intensity of the western blots semi-quantitatively. Our results indicated that blots probed with p-tau217, p-tau231, p-tau404 and p-tau422 antibodies demonstrated high contrast of aggregate intensities comparing AD brains versus control brains. P-tau181, p-tau202, p-tau205, and p-tau262 showed less contrast in HMW aggregate intensities for AD and control brains.

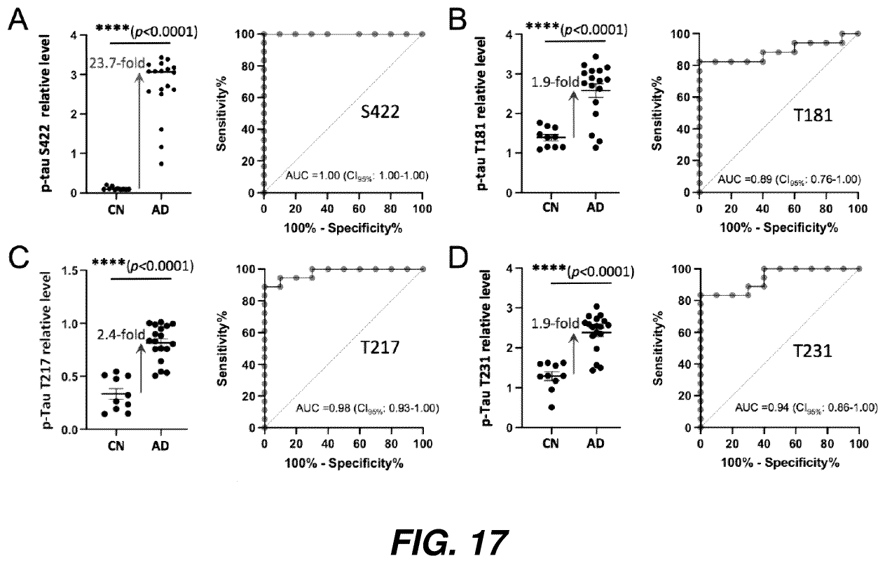

P-tau S422 as a histopathological biomarker for AD detection that outcompetes several existing p-tau biomarkers. P-tau S422 was identified from our antibody screening assays as an epitope with one of the highest contrasts of p-tau aggregates between AD versus control brains. We therefore selected this site for further new biomarker characterization and validation. We quantified p-tau422 levels in the brain homogenates of temporal cortex in the discovery cohort using ELISA assays. Brain p-tau S422 levels were significantly increased in AD brains when compared with controls (2.65±0.18 vs. 0.11±0.013 relative units with an exceptional 23.7-fold increase and a p value of 6.7×10⁻¹¹; Highlighted as red arrow; FIG. 17, panel A). Diagnostic performance sensitivity and selectivity to identify AD cases from normal controls achieved perfect scores (AUC=1.00 and CI_{95%}=1.00-1.00).

In comparison to several existing p-tau biomarkers T181, T217, and T231, p-tau422 showed better differentiating power than all three biomarkers to identify AD from controls. P-tau181 showed differentiating power with AUC=0.89 and CI_{95%}=0.76-1.00. Average p-tau181 concentrations were 2.58±0.17 relative units in the AD brains and 1.39±0.08 relative units in the control brains, a 1.9-fold increase with a p value of 2.49×10⁻⁶ (FIG. 17, panel B). P-tau217 showed differentiating power to identify AD from controls with AUC=0.98 and CI_{95%}=0.93-1.00. Average p-tau217 concentrations were 0.81±0.04 relative units in the AD brains and 0.33±0.05 relative units in the control brains, a 2.4-fold increase with a p value of 3.68×10⁻⁷ (FIG. 17, panel C). P-tau231 showed differentiating power to identify AD from controls with AUC=0.94 and CI95%=0.86-1.00. Average p-tau231 concentrations were 2.39±0.11 relative units in the AD brains and 1.29±0.11 relative units in the control brains, a 1.9-fold increase with a p value of 3.86×10⁻⁷ (FIG. 17, panel D).

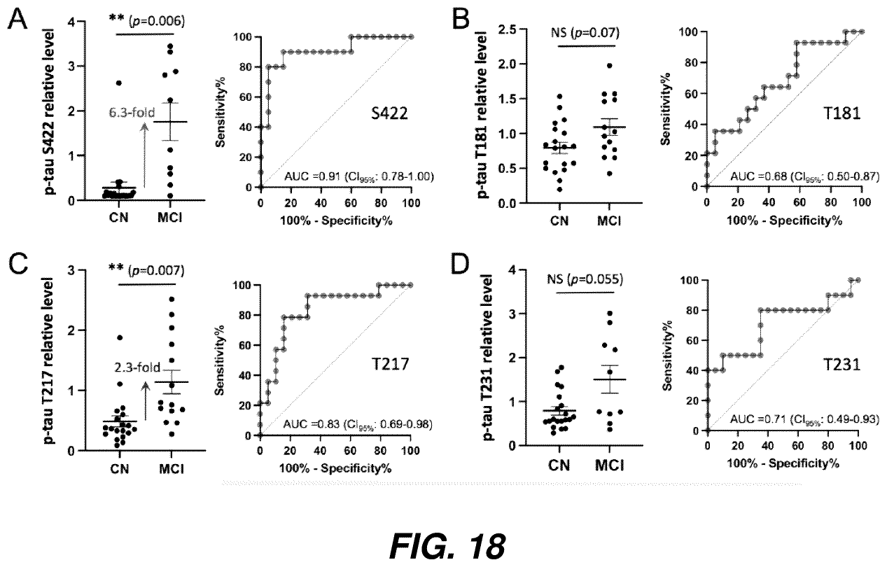

P-tau S422 as a novel biomarker for detecting mild cognitive impairment in brain. To test the potential of p-tau422 as a brain biomarker for more challenging MCI differential detection, we quantified p-tau422 levels in the brain homogenates of temporal cortex in the discovery cohorts of MCI and cognitively normal subjects using ELISA assays. Brain p-tau S422 levels dramatically increased in MCI brains when compared with cognitively normal brains (1.76±0.41 vs. 0.28±0.02 relative units with an exceptional 6.3-fold increase and a p-value of 0.006; FIG. 18, panel A). This is the highest level of increase we observed among all the p-tau epitopes we screened. P-tau422 was able to discriminate MCI brains from cognitively normal controls with outstanding diagnostic scores of AUC value of 0.91 and CI_{95%}=0.78-1.00. In contrast, two known p-tau biomarkers p-tau181 and p-tau231 were unable to differentiate MCI from cognitively normal brains: p-tau181 and p-tau231 levels in MCI brains were not significantly elevated in comparison with those of cognitively normal brains (p=0.07 and p=0.055 respectively; FIG. 18, panels B and D). P-tau217 was able to differentiate MCI from normal control brains; p-tau217 levels increased significantly in MCI brains when compared with cognitively normal brain with a significant 2.3-fold increase and a p-value of 0.007 (FIG. 18, panel C). ROC plots showed strong discriminating power for p-tau217 marker (AUC=0.83 and CI_{95%}=0.69-0.98) and fair and low discriminating power for p-tau181 marker (AUC=0.68 and CI_{95%}=0.50-0.87) and p-tau231 marker (AUC=0.71 and CI_{95%}=0.49-0.93). Therefore, p-tau422 outcompetes all three biomarkers, p-tau181, p-tau217, and p-tau231 in detecting MCI from cognitively normal brains. Prior to this work and to the best of our knowledge, there are no established biomarkers for diagnosing MCI.

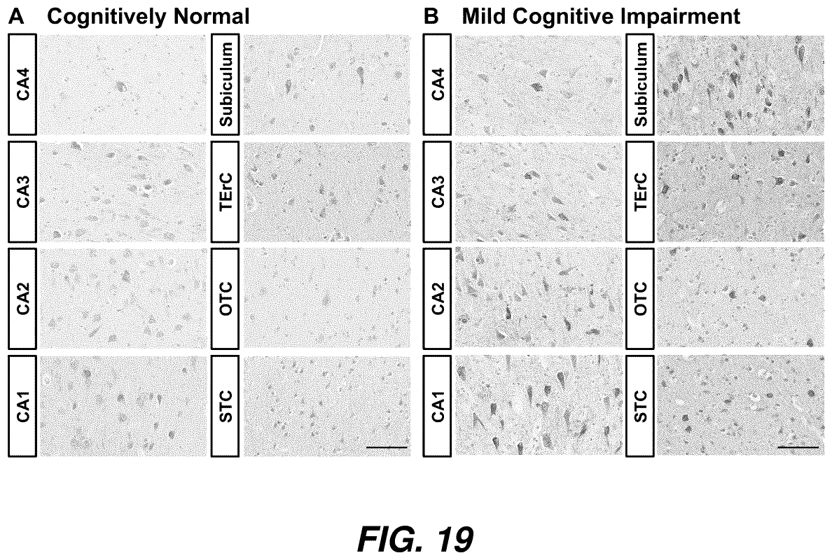

P-tau S422 IHC-derived tau burden correlates with antemortem cognitive status. To examine whether p-tau422 can be used to differentiate participants with MCI from those that are cognitively normal on postmortem neuropathological analysis, sections of the hippocampus and STC from a subset of our cohort were immunostained for p-tau422. Overall, p-tau422 showed a higher density of neurofibrillary tangles (NFT) in participants with MCI compared to cognitively normal controls (FIG. 19).

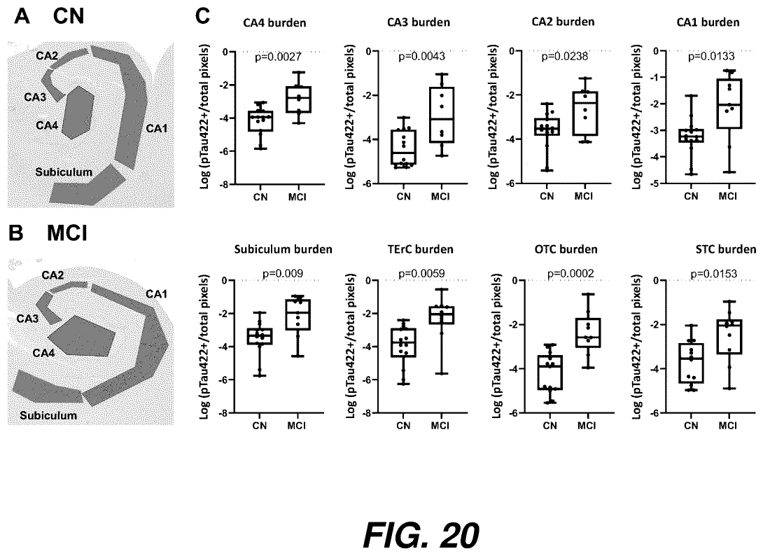

Recent studies have shown that neurofibrillary tangle burden derived from artificial intelligence (AI)-driven image analysis was associated with antemortem cognitive impairment.²⁴ We applied machine learning algorithms using the Visiopharm Oncotopix Discovery software to quantify p-tau422-positive pixels from whole slide images of brain sections immunostained for p-tau422. P-tau422-positive pixels were first annotated manually on training set images and the software was trained to identify positive pixels using a linear Bayesian regression algorithm. Regions of interest (ROI), including the hippocampus CA4, CA3, CA2, CA1, subiculum, TErC, OTC, and STC were manually segmented (FIG. 20, panels A and B) on test set images, and the total number of p-tau422-positive pixels and total pixels in each ROI was automatically calculated. Tau burden was defined as the ratio of p-tau422-positive pixels over total pixels of each ROI. Tau burden was seen to be significantly higher in the CA4, CA3, CA2, CA1, subiculum, TErC, OTC, and STC of participants with MCI over cognitively normal participants (FIG. 20, panel C).

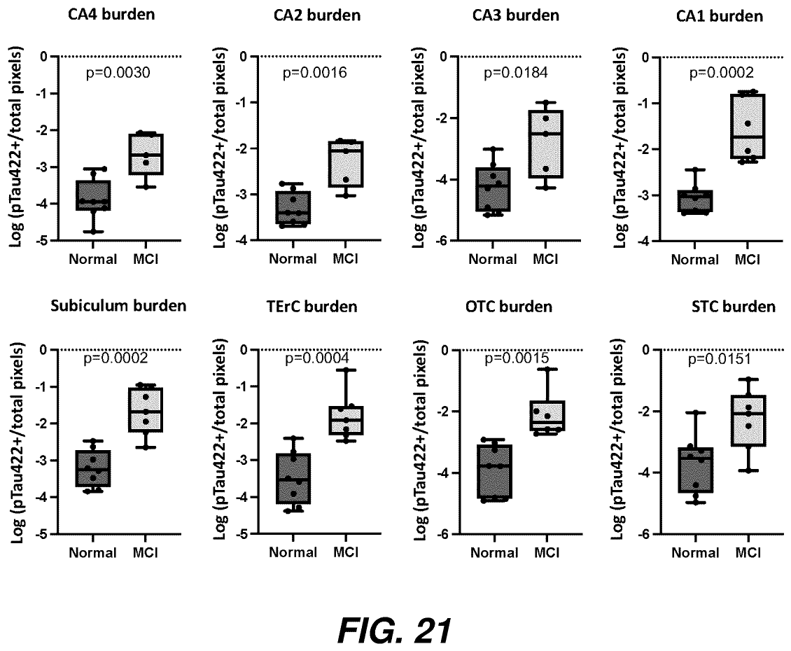

While Braak staging is the most widely used staging system to assess tau pathology in routine neuropathological diagnosis, Braak stage focuses on the distribution of NFT's, and individuals with normal cognition and MCI can have significant overlap Braak stages.^{25,26} To test whether AI-derived tau burden based on p-tau422 IHC correlates with antemortem cognitive status in subjects with similar Braak stages, we repeated the above analysis on participants that are Braak stage III or IV. Again, tau burden was significantly higher in the hippocampus CA4, CA3, CA2, CA1, subiculum, TErC, OTC, and STC of participants with MCI over cognitively normal participants (FIG. 21).

### DISCUSSION

To the best of our knowledge, p-tau422 has not been identified and characterized in the past as a novel brain biomarker for MCI detection. This new utility is consistent with past site-specific p-tau epitope studies. Hyman and colleagues studied 11 phosphorylation dependent tau antibodies and a panel of AD cases of varying severity on three stages of neurofibrillary tangle development and found relatively early stage of intra-neuronal neurofibrillary tangle was prominently stained with p-tau422 whereas relatively late stage of extracellular NFTs were most prominently stained with AT8 (p-taul99/p-tau202/p-tau205) and PHF-1 (p-tau396/p-tau404) antibodies.²⁷ AT8 antibody is routinely used as an AD histopathological biomarker for neuropathological diagnosis. We expect p-tau422 will be a valuable new tool complementary to AT8 antibody, especially for neuropathological diagnosis of early AD.

Identification of p-tau422 or other promising candidates for MCI detection is highly significant in providing important leads for early disease diagnosis. MCI represents a transitional state between the cognitive changes of normal aging and very early dementia. Clinically it is often challenging to differentiate MCI from cognitively normal subjects. Yet, early detection of this MCI stage of AD is important for effective medical intervention to slow down or prevent AD progression. High-molecular-weight oligomers of tau (HMW-tau) in postmortem AD brains have been described in two recent reports.^{28,29} However, neither systematic screens of using such HMW-tau aggregates as readouts nor any screens targeting phosphorylation sites with high AD patient frequencies have been performed and/or reported. Our work independently discovered AD-specific HMW-tau oligomers as “signal reporter” and we validated our approach with three known p-tau biomarkers. A few p-tau biomarkers showed some potential for differentiating A3+MCI cohorts from Aβ− cognitively normal controls. Plasma p-tau181 levels were found to be elevated in A3+ cognitively unimpaired, Aβ+ MCI and Aβ+ AD dementia than in Aβ-cognitively unimpaired and non-AD disease groups.⁷ We recently showed that Simoa p-tau181 levels were capable to differentiate MCI from cognitively normal cohorts with matching plasma and CSF samples.³⁰ Plasma p-tau217 levels were increased during the early preclinical stages of AD when insoluble tau aggregates were not yet detectable by tau-PET, therefore plasma p-tau217 holds promise as a biomarker for early AD brain pathology.³¹ CSF p-tau231 was sensitive to the earliest changes of Ab in the medial orbitofrontal, precuneus and posterior cingulate before global Ab PET positivity was reached.³² CSF p-tau235 levels were elevated in amyloid-positive MCI cases from amyloid-negative cognitively unimpaired cohorts.²³ However, p-tau422 described in this study demonstrated extraordinary diagnostic performance characteristics including superior sensitivity and selectivity, in comparison with known p-tau markers p-tau181, p-tau217, and p-tau231.

Several lines of evidence indicate that phosphorylation of S422 may be related to early events of molecular pathogenesis in AD. P-tau422 site has been described as a disease-specific phospho-epitope associated with tau misfolding.^{33,34} It plays prominent roles in the early stages of AD pathogenesis and persists until late-stage disease and was considered as an attractive target for antibody therapeutics.³⁵⁻³⁸ S422 is involved in NFT formation on phosphorylation in both in vitro and in vivo experiments. Although Ab may not be correlative with degeneration, there is a link between fibrillation of Ab42 and the induction of phosphorylation at S422 in the amygdala.³⁹ Similarly, in a pro-aggregation (P301L) model of SH-SY5Y cells, Ab42 induction increased insoluble tau and PHF-like filaments with P301L and P301L/S422E tau, a pseudo-phosphorylated form, but not P301L/S422A tau, an unphosphorylated form, of tau at S422.⁴⁰

Over ninety PTMs sites in tau protein have been identified, which include dozens of phosphorylation sites, and over a dozen each of acetylation sites and ubiquitination sites.¹²,1³ Based on sequential accumulation model, different tau PTMs occur at different AD stages and specific combinations of PTMs were proposed to reflect progressive steps in the process of tau fibril formation and AD disease progression.^{11,12} Therefore, Tau PTMs sites provided rich opportunities for antibody-based site-specific biomarker discovery, including biomarkers for early disease stages, which are urgent unmet needs. In additional to novel p-tau epitopes, acetylated and ubiquitinated tau epitopes have the potential to serve as molecular markers to distinguish AD from primary tauopathies.⁴¹ Identification of p-tau422 or other MCI biomarkers therefore are significant not only in providing better neuropathological markers (for example, more specific and selective than classical neuropathological p-tau marker AT8), but also potentially important leads for early disease diagnosis. Given that phosphorylation and other PTMs are a common theme implicated in disease pathogenesis and progression in multiple neurodegenerative diseases relevant to misfolding-prone proteins including tau, α-synuclein, and TDP-43, we envision that our biomarker discovery approach may be extended more broadly to other neurodegenerative diseases for detection and diagnosis.^{12,13,42-44}

### REFERENCES FOR EXAMPLE 4

1. Braak H, Braak E. Neuropathological staging of Alzheimer-related changes. Acta Neuropathol. 1991; 82:239-259.
2. Selkoe D J. Alzheimer's disease: genes, proteins, and therapy. Physiol Rev. 2001; 81:741-766.
3. Spillantini M G, Goedert M. Tau protein pathology in neurodegenerative diseases. Trends Neurosci. 1998; 21:428-433.
4 3. Wilcock G K, Esiri M M. Plaques, tangles and dementia. A quantitative study. J Neurol Sci. 1982; 56:343-356.
5. Nelson P T, Alafuzoff I, Bigio E H, et al. Correlation of Alzheimer disease neuropathologic changes with cognitive status: a review of the literature. J Neuropathol Exp Neurol. 2012; 71:362-381.
6. Grundman M. Mild cognitive impairment can be distinguished from alzheimer disease and normal aging for clinical trials. Arch. Neurol. 2004; 61:59-66.
7. Blennow K, Dubois B, Fagan A M, et al. (2015) Clinical utility of cerebrospinal fluid biomarkers in the diagnosis of early Alzheimer's disease. Alzheimers Dement. 2015; 11:58-69.
8. Janelidze S, Mattsson N, Palmqvist S, et al. (2020) Plasma P-tau181 in Alzheimer's disease: relationship to other biomarkers, differential diagnosis, neuropathology and longitudinal progression to Alzheimer's dementia. Nat Med. 2020; 26:379-386.
9. Thijssen E H, La Joie R, Strom A, et al. Plasma phosphorylated tau 217 and phosphorylated tau 181 as biomarkers in Alzheimer's disease and frontotemporal lobar degeneration: a retrospective diagnostic performance study. Lancet Neurol. 2021; 20:739-752.
10. Ashton N J, Pascoal T A, Karikari T K, et al. Plasma p-tau231: a new biomarker for incipient Alzheimer's disease pathology. Acta Neuropathol. 2021; 141:709-724.
11. Leuzy A, Mattsson-Carlgren N, Palmqvist S, et al. Blood-based biomarkers for Alzheimer's disease. EMBO Mol Med. 2022; 14:e14408.
12. Wesseling H, Mair W, Kumar M, et al. Tau PTM Profiles Identify Patient Heterogeneity and Stages of Alzheimer's Disease. Cell. 2020; 183:1699-1713.e13.
13. Arakhamia T, Lee C E, Carlomagno Y, et al. Posttranslational Modifications Mediate the Structural Diversity of Tauopathy Strains. Cell. 2020; 180:633-644.e12.
14. Dujardin S, Commins C, Lathuiliere A, et al. Tau molecular diversity contributes to clinical heterogeneity in Alzheimer's disease. Nat Med. 2020; 26:1256-1263.
15. Hulette C M, Welsh-Bohmer K A, Murray M G, et al. Neuropathological and neuropsychological changes in “normal” aging: evidence for preclinical Alzheimer disease in cognitively normal individuals. J Neuropathol Exp Neurol. 1998; 57:1168-1174.
16. Hyman B T, Phelps C H, Beach T G, et al. National Institute on Aging-Alzheimer's Association guidelines for the neuropathologic assessment of Alzheimer's disease. Alzheimers Dement. 2012; 8:1-13
17. McKeith I G, Boeve B F, Dickson D W, et al. Diagnosis and management of dementia with Lewy bodies: Fourth consensus report of the DLB Consortium. Neurology. 2017; 89:88-100.
18. Cairns N J, Bigio E H, Mackenzie I R, et al. Neuropathologic diagnostic and nosologic criteria for frontotemporal lobar degeneration: consensus of the Consortium for Frontotemporal Lobar Degeneration. Acta Neuropathol. 2007; 114:5-22.
19. Nelson P T, Dickson D W, Trojanowski J Q, et al. Limbic-predominant age-related TDP-43 encephalopathy (LATE): consensus working group report. Brain. 2019; 142:1503-1527.
20. Wu L, Gilyazova N, Ervin J F, et al. Site-specific phospho-tau aggregation-based biomarker discovery for AD diagnosis and differentiation. ACS Chem. Neurosci. 2022; 13:3281-3290
21. Suárez-Calvet M, Karikari T K, Ashton N J, et al. (2020) Novel tau biomarkers phosphorylated at T181, T217 or T231 rise in the initial stages of the preclinical Alzheimer's continuum when only subtle changes in Aβ pathology are detected. EMBO Mol Med. 2020; 12:e12921.
22. Karikari T K, Emeršič A, Vrillon A, et al. Head-to-head comparison of clinical performance of CSF phospho-tau T181 and T217 biomarkers for Alzheimer's disease diagnosis. Alzheimers Dement. 2021; 17:755-767.
23. Lantero-Rodriguez J, Snellman A, Benedet A L, et al. P-tau235: a novel biomarker for staging preclinical Alzheimer's disease. EMBO Mol Med. 2021; 13:e15098.
24. Marx G A, Koenigsberg D G, McKenzie A T, et al. Artificial intelligence-derived neurofibrillary tangle burden is associated with antemortem cognitive impairment. Acta Neuropathol Commun. 2022; 10:157.
25. Mufson E J, Malek-Ahmadi M, Perez S E, et al. Braak staging, plaque pathology, and APOE status in elderly persons without cognitive impairment. Neurobiol Aging. 2016; 37:147-153.
26. Petersen R C, Parisi J E, Dickson D W, et al. Neuropathologic features of amnestic mild cognitive impairment. Arch Neurol. 2006; 63:665-672.
27. Augustinack J C, Schneider A, Mandelkow E M, et al. Specific tau phosphorylation sites correlate with severity of neuronal cytopathology in Alzheimer's disease. Acta Neuropathol. 2002; 103:26-35.
28. Zhou Y, Shi J, Chu D, et al. Relevance of Phosphorylation and Truncation of Tau to the Etiopathogenesis of Alzheimer's Disease. Front Aging Neurosci. 2018; 10:27.
29. Miao J, Shi R, Li L, et al. Pathological Tau From Alzheimer's Brain Induces Site-Specific Hyperphosphorylation and SDS- and Reducing Agent-Resistant Aggregation of Tau in vivo. Front Aging Neurosci. 2019; 11:34.
30. Wu L, Arvai S, Wang S J, et al. Differential Diagnosis of Mild Cognitive Impairment of Alzheimer's Disease by Simoa p-tau181 Measurements with Matching Plasma and CSF. Submitted.
31. Janelidze S, Berron D, Smith R, et al. Associations of plasma phospho-tau217 levels with tau positron emission tomography in early Alzheimer disease. JAMA Neurol 2021; 78:149-156
32. Ashton N J, Benedet A L, Pascoal T A, et al. Cerebrospinal fluid p-tau231 as an early indicator of emerging pathology in Alzheimer's disease. EBioMedicine. 2022; 76:103836
33. Bussière T, Hof P R, Mailliot C, et al. Phosphorylated serine422 on tau proteins is a pathological epitope found in several diseases with neurofibrillary degeneration. Acta Neuropathol. 1999; 97:221-230.
34. Guillozet-Bongaarts A L, Cahill M E, Cryns V L, et al. Pseudophosphorylation of tau at serine 422 inhibits caspase cleavage: in vitro evidence and implications for tangle formation in vivo. J Neurochem. 2006; 97:1005-1014.
35. Vana L, Kanaan N M, Ugwu I C, et al. Progression of tau pathology in cholinergic Basal forebrain neurons in mild cognitive impairment and Alzheimer's disease. Am J Pathol. 2011; 179:2533-2550.
36. Troquier L, Caillierez R, Burnouf S, et al. Targeting phospho-Ser422 by active Tau Immunotherapy in the THYTau22 mouse model: a suitable therapeutic approach. Curr Alzheimer Res. 2012; 9:397-405.
37. Collin L, Bohrmann B, Göpfert U, et al. Neuronal uptake of tau/pS422 antibody and reduced progression of tau pathology in a mouse model of Alzheimer's disease. Brain. 2014; 137(Pt 10):2834-2846.
38. Voss K, Koren J 3^{rd}, Dickey C A. The earliest tau dysfunction in Alzheimer's disease?Tau phosphorylated at s422 as a toxic seed. Am J Pathol. 2011; 179:2148-2151.
39. Götz J, Chen F, van Dorpe J, et al. Formation of neurofibrillary tangles in P3011 tau transgenic mice induced by Abeta 42 fibrils. Science. 2001; 293:1491-1495.
40. Ferrari A, Hoerndli F, Baechi T, et al. beta-Amyloid induces paired helical filament-like tau filaments in tissue culture. J Biol Chem. 2003; 278:40162-40168.
41. Kyalu Ngoie Zola N, Balty C, Pyr Dit Ruys S, et al. Specific post-translational modifications of soluble tau protein distinguishes Alzheimer's disease and primary tauopathies. Nat Commun. 2023; 14:3706.
42. Schmid A W, Fauvet B, Moniatte M, et al. Alpha-synuclein posttranslational modifications as potential biomarkers for Parkinson disease and other synucleinopathies. Mol Cell Proteomics. 2013; 12:3543-3558.
43. Manzanza N O, Sedlackova L, Kalaria R N. Alpha-Synuclein Posttranslational Modifications: Implications for Pathogenesis of Lewy Body Disorders. Front Aging Neurosci. 2021; 13:690293.
44. Frangçis-Moutal L, Perez-Miller S, Scott D D, et al. Structural Insights Into TDP-43 and Effects of Posttranslational Modifications. Front Mol Neurosci. 2019; 12:301.
45. Rissin D M, Kan C W, Campbell T G, et al. Single-molecule enzyme-linked immunosorbent assay detects serum proteins at subfemtomolar concentrations. Nat Biotechnol. 2010; 28:595-599.
46. Yang T, O'Malley T T, Kanmert D, et al. A highly sensitive novel immunoassay specifically detects low levels of soluble Aβ oligomers in human cerebrospinal fluid. Alzheimer's Res Ther. 2015; 7:14.
47. Barthélemy NR, Horie K, Sato C, Bateman R J. Blood plasma phosphorylated-tau isoforms track CNS change in Alzheimer's disease. J Exp Med. 2020; 217:e20200861.
48. Karikari T K, Ashton N J, Brinkmalm G, et al. Blood phospho-tau in Alzheimer disease: analysis, interpretation, and clinical utility. Nat Rev Neurol. 2022; 18:400-418.

The foregoing is illustrative of the present inventive concept and is not to be construed as limiting thereof. Further embodiments of the present inventive concept are exemplified in the following claims.

That which is claimed:

1. A method of diagnosing a neurodegenerative disease/disorder in a subject comprising:
determining an extent of phosphorylation and/or hyperphosphorylation of at least one phospho-tau (p-tau) biomarker in the subject; and
diagnosing whether the subject is afflicted with the neurodegenerative disease/disorder if the extent of phosphorylation and/or hyperphosphorylation of the p-tau biomarker exceeds a threshold indicative of a presence of the neurodegenerative disease/disorder.

2. The method of claim 1, wherein the neurodegenerative disease/disorder is a tauopathy.

3. The method of claim 2, wherein the tauopathy is selected from the group consisting of Alzheimer's disease (AD), Pick's disease (PiD), progressive supranuclear palsy (PSP), and corticobasal degeneration (CBD).

4. The method of claim 3, wherein the tauopathy is AD.

5. The method of claim 1, wherein the p-tau biomarker is selected from the group consisting of p-tauS198, p-tauT212/S214, p-tauS262/T263, p-tauS356, p-tauS396, and p-tauS422, or a combination of one or more of any thereof.

6. The method of claim 5, wherein the p-tau biomarker is p-tau198, p-tauT212/S214, p-tauS262/T263, p-tauS356, p-tauS396, or p-tauS422.

7. The method of claim 5, wherein the p-tau biomarker is p-tauS198, p-tauS356, p-tauS396, or p-TauS422.

8. A method of treating a neurodegenerative disease/disorder in a subject comprising:
diagnosing for a neurodegenerative disease/disorder in a subject with the method of claim 1; and
treating the subject for the neurodegenerative disorder if it is determined from the diagnosing that the subject is afflicted with the neurodegenerative disorder.

9. A method of differentiating a neurodegenerative disease/disorder in a subject comprising:
determining an extent of phosphorylation and/or hyperphosphorylation of at least one p-tau biomarker; and
differentiating whether the subject is afflicted with the neurodegenerative disease/disorder based on if the extent of phosphorylation and/or hyperphosphorylation of the p-tau biomarker exceeds a threshold indicative of the neurodegenerative disease/disorder.

10. The method of claim 9, wherein the neurodegenerative disease/disorder is a tauopathy.

11. The method of claim 10, wherein the tauopathy is selected from the group consisting of Alzheimer's disease (AD), Pick's disease (PiD), progressive supranuclear palsy (PSP), and corticobasal degeneration (CBD).

12. The method of claim 11, wherein the tauopathy is AD.

13. The method of claim 9, wherein the p-tau biomarker is selected from the group consisting of p-tauS198, p-tauT212/S214, p-tauS262/T263, p-tauS356, p-tauS396, and p-tauS422, or a combination of one or more of any thereof.

14. The method of claim 13, wherein the p-tau biomarker is p-tau198, p-tauT212/S214, p-tauS262/T263, p-tauS356, p-tauS396, or p-tauS422.

15. The method of claim 13, wherein the p-tau biomarker is p-tauS198, p-tauS356, p-tauS396, or p-tauS422.

16. A method of treating a neurodegenerative disease/disorder in a subject in need thereof comprising:
determining the neurodegenerative disease/disorder that the subject is afflicted with the method of claim 9; and
treating the subject for the neurodegenerative disease/disorder the subject is determined to be afflicted with.

17. A method of diagnosing mild cognitive impairment (MCI) in a subject comprising:
determining an extent of phosphorylation and/or hyperphosphorylation of at least one p-tau biomarker; and
diagnosing whether the subject is afflicted with MCI if the extent of phosphorylation and/or hyperphosphorylation of the p-tau biomarker exceeds a threshold indicative of the subject being afflicted with MCI.

18. The method of claim 17, wherein the p-tau biomarker is selected from the group consisting of p-tauS198, p-tauT212/S214, p-tauS262/T263, p-tauS356, p-tauS396, and p-tauS422, or a combination of one or more of any thereof.

19. The method of claim 17, wherein the p-tau biomarker is p-tau198, p-tauT212/S214, p-tauS262/T263, p-tauS356, p-tauS396, or p-tauS422.

20. The method of claim 17, wherein the p-tau biomarker is p-tauS198, p-tauS356, p-tauS396, or p-tauS422.

21. A method of treating mild cognitive disorder (MCI) comprising:
diagnosing for MCI in a subject with the method of claim 17; and
treating the subject for MCI if it is determined from the diagnosing that the subject is afflicted with MCI.

22. A method for differentiating MCI from normal cognitive decline in a subject comprising:
determining an extent of phosphorylation and/or hyperphosphorylation of a p-tau biomarker; and
differentiating whether the subject is afflicted with MCI from normal cognitive decline based on if the extent of phosphorylation and/or hyperphosphorylation of the p-tau biomarker exceeds a threshold indicative of the subject being afflicted with MCI.

23. The method of 22, wherein the p-tau biomarker is selected from the group consisting of p-tauS198, p-tauT212/S214, p-tauS262/T263, p-tauS356, p-tauS396, and p-tauS422, or a combination of one or more of any thereof.

24. The method of claim 23, wherein the p-tau biomarker is p-tau198, p-tauT212/S214, p-tauS262/T263, p-tauS356, p-tauS396, or p-tauS422.

25. The method of claim 23, wherein the p-tau biomarker is p-tauS198, p-tauS356, p-tauS396, or p-tauS422.
